(19) United States
(12) Patent Application Publication
(10) Pub. No.: US 2026/0152533 A1
(43) Pub. Date: Jun. 4, 2026

(54) COMBINATION INHIBITORS FOR TREATING FIBROSIS

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US)

(72) Inventors: Craig B. Thompson, New York, NY (US); Simon Schwoerer, New York, NY (US)

(73) Assignees: Memorial Sloan-Kettering Cancer Center, New York, NY (US); Sloan-Kettering Institute for Cancer Research, New York, NY (US); Memorial Hospital for Cancer and Allied Diseases, New York, NY (US)

(21) Appl. No.: 18/706,785

(22) PCT Filed: Nov. 3, 2022
(86) PCT No.: PCT/US2022/079243

§ 371 Date: May 2, 2024

Related U.S. Application Data
(60) Provisional application No. 63/275,056, filed on Nov. 3, 2021.

(51) Int. Cl. C07K 14/495; A61K 38/00

(52) U.S. Cl. CPC C07K 14/495; A61K 38/00

(57) ABSTRACT

Aspects of the present disclosure relate to the discovery that antagonizing pyruvate carboxylase and glutaminase inhibits cell proliferation and extracellular matrix production. Thus, methods and compositions disclosed herein may be used to treat fibrotic disease and cancer by inhibiting cell proliferation and extracellular matrix production.

### CROSS-REFERENCE TO RELATED APPLICATIONS

This application claims priority under 35 U.S.C. § 119 (e) to U.S. Provisional Patent Application No. 63/275,056, filed Nov. 3, 2021, which is hereby incorporated by reference in its entirety.

### GOVERNMENT SUPPORT

This invention was made with government support under CA259224 and CA201318 awarded by the National Institutes of Health. The government has certain rights in this invention.

### REFERENCE TO AN ELECTRONIC SEQUENCE LISTING

The contents of the electronic sequence listing (S171570053WO00-SEQ-EMB.xml; Size: 8,582 bytes; and Date of Creation: Nov. 3, 2022) is herein incorporated by reference in its entirety.

### BACKGROUND

The aberrant production of collagen by fibroblasts is a hallmark of (i) many solid tumors and can influence cancer progression; and (ii) fibrotic disorders. Fibroblasts use glutamine and glucose for the synthesis of non-essential amino acids required for extracellular matrix protein synthesis. How the cells in the tumor microenvironment maintain their production of extracellular matrix proteins as the vascular delivery of glutamine and glucose becomes compromised has remained unclear.

### SUMMARY

The present disclosure is based on the surprising discovery that pyruvate carboxylase (PC) activity is required for cell (e.g., fibroblast, tumor cell) proliferation and collagen production in a nutrient-deficient environment. Unexpectedly, antagonizing PC activity and glutaminase activity in a nutrient-replete environment is synergistic compared with antagonizing PC activity or glutaminase activity singly. Therefore, the present disclosure provides methods and compositions for inhibiting cell proliferation and collagen production in a nutrient-deficient and nutrient-replete environment.

In some aspects, the present disclosure provides a method of inhibiting collagen synthesis by a fibroblast comprising contacting the fibroblast with an antagonist of pyruvate carboxylase (PC) and an antagonist of glutaminase.

In some aspects, the present disclosure provides a method of treating a fibrotic disorder, the method comprising: administering to a subject in need thereof an antagonist of PC in an amount effective to inhibit to inhibit extracellular matrix (ECM) protein production, wherein the subject is also receiving an antagonist of glutaminase.

In some aspects, the present disclosure provides a method of treating a fibrotic disorder, the method comprising: administering to a subject in need thereof an antagonist of glutaminase in an amount effective to inhibit to inhibit extracellular matrix (ECM) protein production, wherein the subject is also receiving an antagonist of PC.

In some aspects, the present disclosure provides a method of treating a fibrotic disorder, the method comprising: administering to a subject in need thereof an antagonist of PC and an antagonist of glutaminase in an amount effective to inhibit ECM production.

In some aspects, the present disclosure provides a method of treating a cancer, the method comprising: administering to a subject in need thereof an antagonist of PC in an amount effective to inhibit carcinogenesis, wherein the subject is also receiving an antagonist of glutaminase.

In some aspects, the present disclosure provides a method of treating a cancer, the method comprising: administering to a subject in need thereof an antagonist of glutaminase in an amount effective to inhibit carcinogenesis, wherein the subject is also receiving an antagonist of PC.

In some aspects, the present disclosure provides a composition comprising an antagonist of PC and an antagonist of glutaminase.

In some aspects, the present disclosure provides a kit comprising an antagonist of PC. an antagonist of glutaminase, and instructions for use of the antagonist of PC and the antagonist of glutaminase.

In some embodiments, the present disclosure provides a method of inhibiting tumor cell proliferation, the method comprising contacting the tumor cell with an antagonist of PC and an antagonist of glutaminase. In some embodiments, inhibition of tumor cell proliferation results from reduction of ECM protein production

In some embodiments, the antagonist of PC is a small molecule, a nucleic acid, a polypeptide, or a protein. In some embodiments, the antagonist of glutaminase is a small molecule, a nucleic acid, a polypeptide, or a protein.

In some embodiments, the fibrotic disorder is characterized by cell hyperproliferation and/or ECM protein hyperproduction. In some embodiments, the fibrotic disorder is characterized by a low-glutamine. In some embodiments, the contacting is in a low-glutamine environment. In some embodiments, carcinogenesis is characterized by a low-glutamine. In some embodiments, the fibrotic disorder is characterized by low-glucose and high-lactate. In some embodiments, the carcinogenesis is characterized by low-glucose and high-lactate. In some embodiments, the contacting is in a low-glucose and a high-lactate environment.

In some embodiments, treating the fibrotic disorder results in amelioration of cell hyperproliferation and/or ECM protein hyperproduction. In some embodiments, the ECM protein is collagen, elastin, and/or laminin. In some embodiments, the fibrotic disorder is pulmonary fibrosis or liver fibrosis.

In some embodiments, the fibroblast is in a low-glutamine environment. In some embodiments, the fibroblast is characterized by cell hyperproliferation and/or collagen hyperproduction. In some embodiments, the antagonist of PC decreases the level of acetylated H3K27 at a collagen gene enhancer. In some embodiments, the fibroblast is associated with pulmonary fibrosis, liver fibrosis, and/or cancer.

In some embodiments, the tumor cell is a fibroblast. In some embodiments, the tumor cell is derived from a carcinoma, a sarcoma, a breast cancer, a pancreatic cancer, a lung cancer, a leukemia, a lymphoma, a brain cancer, a melanoma, a liver cancer, a stomach cancer, a small intestine cancer, a large intestine cancer, a kidney cancer, a uterine cancer, an ovarian cancer, a bladder cancer, or a bone cancer.

In some embodiments, the cancer is characterized by cell hyperproliferation and/or ECM protein hyperproduction. In some embodiments, treating the cancer results in a reduction of cell proliferation and/or ECM matrix protein production.

In some embodiments, the contacting is in vitro. In some embodiments, the contacting is in vivo.

In some embodiments, methods provided herein further comprise administering an antagonist of glutaminase to the subject. In some embodiments, methods provided herein further comprise administering an antagonist of PC to the subject. In some embodiments, administering the antagonist of PC and the antagonist of glutaminase is sequential.

In some embodiments, the composition further comprises low-glutamine cell culture medium. In some embodiments, the composition further comprises low-glucose and high-lactate cell culture medium.

In some embodiments, the kit comprises a single container containing the antagonist of PC and the antagonist of glutaminase. In some embodiments, the single container is a vial or a syringe. In some embodiments, the kit comprises a first container containing the antagonist of PC and a second container containing the antagonist of glutaminase. In some embodiments, the first container and the second container are vials or syringes. In some embodiments, the instructions are instructions for use of the antagonist of PC and the antagonist of glutamine in any method provided herein.

The details of certain embodiments of the invention are set forth in the Detailed Description, as described below. Other features, objects, and advantages of the invention will be apparent from the Examples, Drawings, and Claims.

### BRIEF DESCRIPTION OF THE DRAWINGS

The accompanying drawings, which constitute a part of this specification, illustrate several embodiments of the invention and together with the description, serve to explain the principles of the invention.

FIGS. 1A-1J. TGFB-induced collagen synthesis is linked to glutamine (Gln)-dependent tricarboxylic acid (TCA) cycle anaplerosis. FIG. 1A, Growth curves of NIH-3T3 cells cultured in the indicated percentage of Gln and treated with TGFB (2 nanograms/milliliter), n=3 biologically independent samples. Dashed line indicates cell number at day 0 (d 0). FIG. 1B, Western blot of NIH-3T3 cells cultured in the indicated percentage of Gln and treated with TGFB for 48 hours. FIG. 1C, Schematic diagram of fibroblast-derived extracellular matrix (ECM) preparation (left); collagen abundance in ECM derived from confluent NIH-3T3 cells cultured in the indicated percentage of Gln and treated with TGFB (right), n=3 biologically independent samples. FIG. 1D, Transfer RNA charging in NIH-3T3 cells cultured in 100% or 10% Gln and treated with TGFB for 48 hours, n=1 independent experiment. FIG. 1E, Western blot of NIH-3T3 cells cultured in 100% or 10% Gln and treated with TGFB for 48 hours. FIG. 1F, Relative translation rate of NIH-3T3 cells cultured in 100% or 10% Gln and treated with TGFB for 48 hours, n=3 biologically independent samples. FIG. 1G, Western blot of NIH-3T3 cells cultured in 10% Gln and treated with TGFβ and CB839 (glutaminase inhibitor) (1 M) for 48 hours. FIG. 1H, Relative metabolite abundance in NIH-3T3 cells cultured in 100% or 10% Gln and treated with TGFB for 48 hours, n=3 biologically independent samples. FIG. 1I, Relative number of NIH-3T3 cells cultured in 10% Gln and treated with TGFB and 0.2, 1 or 5 mM of the indicated cell-permeable metabolites for 3 days, m-Pro is L-proline-methylester; dm-Glu is L-glutamic acid dimethylester; dm-aKG is dimethyl alpha-ketoglutarate, n=3 biologically independent samples. FIG. 1J, Western blot of NIH-3T3 cells cultured in 10% Gln and treated with TGFB and the indicated concentrations of cell-permeable metabolites for 48 hours. Mean±standard deviation (SD) (FIGS. 1A and 1C) or mean±SD (FIGS. 1F and 1I) are shown. Two-way analysis of variance (ANOVA) (FIGS. 1A and 1C), two-way ANOVA with Holm-Sidak correction (FIG. 1F), one-way ANOVA with Holm-Sidak correction (FIG. 1I). Western blots are representative of two (FIGS. 1B, 1G, and 1J) or three (FIG. 1E) independent experiments, tRNA charging analysis (FIG. 1D) was performed once for 100% Gln. and is representative of three independent experiments for 10% Gln. All other experiments were performed at least twice.

FIGS. 2A-2J. Glutamine de novo synthesis can maintain translation and collagen production when extracellular glutamine is limited. FIG. 2A, tRNA charging in NIH-3T3 cells cultured in 10% Gln and treated with TGFβ (2 nanograms/milliliter), dm-αKG (5 mM) and methionine sulfoximine (MSO; glutamine synthetase inhibitor) (2 mM) for 48 hours, n=1 independent experiment. FIG. 2B. Relative translation rate of NIH-3T3 cells cultured in 10% Gln and treated with TGFβ and dm-αKG and MSO, n=3 biologically independent samples. FIG. 2C. Western blot of NIH-3T3 cells cultured in 10% Gln and treated with TGFβ, dm-Glu (5 mM), dm-αKG and MSO for 48 hours. FIG. 2D. Western blot of ECM produced by confluent NIH-3T3 cells cultured in 100% or 10% Gln, treated with TGFβ and dm-αKG and MSO. FIG. 2E. Collagen abundance in ECM produced by confluent NIH-3T3 cells cultured in 10% Gln, treated with TGFβ, dm-αKG and MSO, n=3 biologically independent samples. FIG. 2F. Western blot of NIH-3T3 cells expressing Ctrl or Glul single guide RNA, cultured in 10% Gln and treated with TGFβ, dm-αKG or dm-Glu for 48 hours. FIG. 2G, tRNA charging in NIH-3T3 cells expressing Ctrl or Glul sgRNA, cultured in 10% Gln for 48 hours, n=1 independent experiments. FIG. 2H. Relative translation rate of NIH-3T3 cells expressing control (Ctrl) or Glul single guide RNA (sgRNA), cultured in 10% Gln for 48 hours, n=3 biologically independent samples. FIG. 2I. Western blot of NIH-3T3 cells expressing Ctrl or Glul sgRNA, cultured in 10% Gln for 48 hours. FIG. 2J. Western blot of NIH-3T3 cells cultured in 100% Gln or 10% Gln and treated with MSO for 48 hours. Mean+SD (FIGS. 2B, 2E and 2H) are shown. One-way ANOVA (FIG. 2H), one-way ANOVA with Holm-Sidak correction (FIG. 2B), two-way ANOVA with Holm-Sidak correction (FIG. 2E). tRNA charging analyses (FIGS. 2A and 2G) are representative of two independent experiments. Western blots (FIGS. 2C, 2D, 2F, 2I, and 2J) are representative of two independent experiments. All other experiments were performed at least twice.

FIGS. 3A-3M. Pyruvate carboxylase (PC) suppression by TGFβ impairs TCA cycle anaplerosis, translation, and collagen production in low glutamine. FIG. 3A, PC (Pcx) mRNA expression in NIH-3T3 cells cultured in 100% Gln and treated with TGFβ for 24 hours, n=3 biologically independent samples. FIG. 3B, Western blot of NIH-3T3 cells cultured in 100% Gln and treated with TGFβ for 48 hours. FIG. 3C, Western blot of NIH-3T3 cells expressing Ctrl or Smad4 sgRNA, cultured in 100% Gln and treated with TGFβ for 48 hours. FIG. 3D, Schematic diagram of [U-¹³C]-glucose (Glc) tracing. FIG. 3E, [U-¹³C]-Glc tracing in NIH-3T3 cells cultured in 100% Gln or 10% Gln and treated with TGFβ for 48 hours, n=3 biologically independent samples. FIG. 3F, Schematic diagram of [3,4-¹³C]-glucose (Glc) tracing. FIGS. 3G and 3H, [3,4-¹³C]-Glc tracing in NIH-3T3 cells cultured in 100% or 10% Gln and treated with TGFβ for 48 hours. M+1 labeling (FIG. 3G), PC activity (FIG. 3H), n=3 biologically independent samples. FIG. 3I, [U-¹³C]-Glc tracing in NIH-3T3 cells expressing empty vector or human PC cDNA cultured in 10% Gln in the presence of TGFβ for 48 hours, n=3 biologically independent samples. FIG. 3J, Relative metabolite abundance in NIH-3T3 cells expressing empty vector or human PC complementary DNA, cultured in 10% Gln in the presence of TGFβ for 48 hours, n=3 biologically independent samples. FIG. 3K, Relative translation rate of NIH-3T3 cells expressing empty vector or human PC cDNA, cultured in 10% Gln in the presence of TGFβ and treated with MSO for 48 hours, n=3 biologically independent samples. FIG. 3L, Growth curve of NIH-3T3 cells expressing empty vector or human PC cDNA, cultured in 10% Gln in the presence of TGFβ, n=3 biologically independent samples. Dashed line indicates cell number at day 0 (d 0). FIG. 3M, Western blot of NIH-3T3 cells expressing empty vector or human PC cDNA and cultured in 10% Gln in the presence of TGFβ for 48 hours. Mean+SD (FIGS. 3A, 3E, 3G-3I, and 3K) or mean±SD (FIG. 3L) are shown. Two-sided unpaired t-test (FIG. 3A), two-way ANOVA with Holm-Sidak correction (FIG. 3E), two-sided unpaired t-test with Holm-Sidak correction (FIG. 3I), one-way ANOVA with Holm-Sidak correction (FIGS. 3G, 3H, and 3K), two-way ANOVA (FIG. 3L). Western blots (FIGS. 3B, 3C, and 3M) are representative of two independent experiments. All other experiments were performed at least twice.

FIGS. 4A-4L. PC anaplerosis is required for collagen production when extracellular glutamine levels are low. FIGS. 4A and 4B, Western blot of NIH-3T3 cells expressing Ctrl or PC sgRNA, cultured in 10% Gln for 48 hours (FIG. 4A) and treated with dm-aKG or dm-Glu for 48 hours (FIG. 4B). FIG. 4C, Relative metabolite abundance in NIH-3T3 cells expressing Ctrl or PC sgRNA, cultured in 10% Gln for 48 hours, n=3 biologically independent samples. FIG. 4D, [U-¹³C]-Glc tracing in NIH-3T3 cells expressing Ctrl or PC sgRNA, cultured in 100% or 10% Gln for 48 hours, n=3 biologically independent samples. FIG. 4E, Western blot of NIH-3T3 cells cultured in 100% Gln and treated with CB839 (1 μM) for 48 hours. FIG. 4F, [U-¹³C]-Glc tracing in NIH-3T3 cells cultured in 100% or 10% Gln and treated with CB839 for 48 hours, n=3 biologically independent samples. FIG. 4G, [3,4-¹³C]-Glc tracing in NIH-3T3 cells cultured in 100% Gln and treated with CB839 for 48 hours, n=3 biologically independent samples. FIG. 4H, Western blot of NIH-3T3 cells expressing Ctrl or PC sgRNA, treated with CB839 for 48 hours. FIG. 4I, Western blot of NIH-3T3 cells expressing Ctrl or PC sgRNA, cultured in 10% Gln and treated with dm-aKG and MSO for 48 hours. FIG. 4J, Gene set enrichment analysis (GSEA) of PC-knockout (PC-ko) compared to control NIH-3T3 cells cultured in 10% Gln, n=2 (Ctrl sg), n=4 (PC-ko) biologically independent samples. FIG. 4K, tRNA charging in NIH-3T3 cells expressing Ctrl or PC sgRNA, cultured in 10% Gln for 48 hours, n=1 independent experiment. FIG. 4L, Relative translation rate of NIH-3T3 cells expressing Ctrl or PC sgRNA, cultured in 10% Gln for 48 hours, n=3 biologically independent samples. Mean+SD (FIGS. 4D, 4F, 4G, and 4L) are shown. Two-way ANOVA with Holm-Sidak correction (FIG. 4D), one-way ANOVA with Holm-Sidak correction (FIG. 4F), two-sided unpaired t-test (FIG. 4G), one-way ANOVA (FIG. 4L). Western blots are representative of three (FIG. 4A) or two (FIGS. 4B, 4E, 4H, and 4I) independent experiments. tRNA charging analyses (FIG. 4K) are representative of two independent experiments. RNA sequencing (FIG. 4J) was performed once. All other experiments were performed at least twice.

FIGS. 5A-5E. Pyruvate carboxylase anaplerosis supports collagen transcription when extracellular glutamine levels are low. FIG. 5A, Col1a1 mRNA expression in NIH-3T3 cells expressing Ctrl or PC sgRNA, cultured in 10% Gln for 48 hours, n=3 biologically independent samples. FIGS. 5B and 5C, H3K27ac (FIG. 5B) or H3K27me3 enrichment (FIG. 5C) in NIH-3T3 cells expressing Ctrl or PC sgRNA, cultured in 10% Gln for 48 h, n=3 (b), n=4 (c) independent experiments. TSS; transcriptional start site. FIG. 5D, Colla1 mRNA expression in NIH-3T3 cells expressing Ctrl or PC sgRNA, cultured in 10% Gln for 48 hours and treated with dm-aKG, n=3 biologically independent samples. FIG. 5E, GSEA in PC-ko compared to control NIH-3T3 cells, n=2 (Ctrl sg), n=4 (PC-ko) biologically independent samples. Mean+SD (FIGS. 5A-5D) are shown. One-way ANOVA (FIGS. 5A, 5B, and 5D), two-way ANOVA (FIG. 5C) analyzing the effects of PC-ko on H3K27me3 across the indicated genomic regions. RNA sequencing (FIG. 5E) was performed once. All other experiments were performed at least twice.

FIGS. 6A-6I. Lactate supports collagen production via PC when glucose and glutamine are limiting. FIG. 6A, Western blot of NIH-3T3 cells cultured in 10% Gln and the indicated concentrations of D-glucose for 48 hours. FIG. 6B, [U-¹³C]-Glc and [U-¹³C]-Lac tracing. NIH-3T3 cells were cultured for 48 hours in 100% or 10% Gln in the presence of 10 mM or 1 mM D-glucose alone or with 10 mM sodium lactate, n=3 biologically independent samples. FIGS. 6C and 6D, [1-¹³C]Lac tracing in NIH-3T3 cells cultured for 48 hours in 100% or 10% Gln in the presence of 10 mM or 1 mM D-glucose and 10 mM sodium lactate, M+1 labelling (FIG. 6C), PC activity (FIG. 6D), n=3 biologically independent samples. FIG. 6E, Western blot of NIH-3T3 cells cultured in 10% Gln and 10 mM or 1 mM D-glucose and treated with sodium lactate or sodium pyruvate for 48 hours. FIG. 6F, Western blot of NIH-3T3 cells cultured in 10% Gln and 20 mM or 2 mM D-glucose for 72 hours and treated with AZD3965 (MCT1 inhibitor, 5 μM) or sodium oxamate (lactate dehydrogenase (LDH) inhibitor, 10 mM). Sodium lactate was added in the last 48 hours. FIG. 6G, [U-¹³C]-Lac tracing. NIH-3T3 cells expressing Ctrl or PC sgRNA were cultured in 10% Gln, 10 mM D-glucose and 10 mM sodium lactate for 48 hours, n=3 biologically independent samples. FIG. 6H, Western blot of NIH-3T3 cells expressing Ctrl or PC sgRNA, cultured in 10% Gln and the indicated concentrations of D-glucose and sodium lactate for 48 hours. FIG. 61, [U-¹³C]-Lac tracing into collagen secreted into the ECM. Confluent NIH-3T3 cells expressing Ctrl or PC sgRNA were cultured in 10% Gln supplemented with [U-¹³C]-Lac for FIG. 6D, n=3 biologically independent samples. Mean+SD (FIGS. 6B-6D, 6G, and 6I) are shown. Two-way ANOVA with Holm-Sidak correction (FIG. 6B), one-way ANOVA (FIGS. 6C, 6G, and 6I). Western blots are representative of two (FIGS. 6A and 6E) or three (FIGS. 6F and 6H) independent experiments. All other experiments were performed at least twice.

FIGS. 7A-7M. Fibroblast PC supports pancreatic and mammary tumor growth and fibrosis. FIG. 7A, Western blot of ECM generated by confluent pancreatic stellate cells (PSCs) expressing Ctrl, PC or Glul sgRNA cultured in 20% Gln. FIGS. 7B and 7C, Outgrowth of KPC (pancreatic ductal adenocarcinoma) spheroids on ECM generated by confluent PSCs PC expressing Ctrl, PC or Glul sgRNA cultured in 20% Gln. Representative images (FIG. 7B) and quantification (FIG. 7C) are shown. Scale bar, 500 pm, n=4 (Ctrl-sg, PC-sg2, Glul-sg6), n=3 (PC-sg5, Glul-sg4) biologically independent samples. FIGS. 7D-7G, KPC cells were injected subcutaneously (s.c.) into nude mice, alone or with PSCs expressing Ctrl, PC or Glul sgRNA. FIG. 7D, Growth curve of KPC/PSC allografts, n=8 biologically independent tumors. FIG. 7E, Representative images of Masson's Trichrome staining of KPC/PSC allografts. Scale bar, 500 pm. FIGS. 7F and 7G, Quantification of Masson's Trichrome (FIG. 7F) or αSMA staining (FIG. 7G) of KPC/PSC allografts, n=8 biologically independent tumors. FIGS. 7H and 71, KPC cells were injected s.c. into syngeneic wildtype mice, alone or with PSCs expressing Ctrl or PC sgRNA. FIG. 7H, Volume of KPC/PSC allografts 8 days after injection, n=9 biologically independent tumors. FIG. 7I, Collagen levels in KPC/PSC allografts 8 days after injection, n=9 biologically independent tumors. FIGS. 7J-7M, DB7 (breast cancer) cells were injected s.c. into wild-type syngeneic mice, alone or with MFBs (spontaneously immortalized cells) expressing Ctrl or PC sgRNA. FIG. 7J, Growth of DB7/MFB allografts, n=7 (d 19 MFB PC-sg2), n=8 (all others) biologically independent tumors. FIG. 7K, Collagen levels in DB7/MFB allografts 8 days after injection, n=8 biologically independent tumors. FIG. 7L, Western blot of DB7/MFB allografts 8 days after injection. FIG. 7M, Collagen 1 band intensity relative to actin from (FIG. 7L) and FIG. 7M. Samples derived from the same experiment and gels/blots were processed in parallel, n=6 (DB7), n=7 (DB7+MFB Ctrl, DB7+MFB PC-ko) biologically independent tumors. Mean±SEM (FIGS. 7C, 7D, and 7J) or median with 25% to 75% percentile box and min/max whiskers (FIGS. 7F-71, 7K, and 7M) are shown. Two-way ANOVA (FIGS. 7C, 7D, and 7J) analyzing the effects of PC-ko or Glul-ko on spheroid or tumor growth over time, one-way ANOVA (FIGS. 7F and 7G), one-way ANOVA with Holm-Sidak correction (FIGS. 7H, 7I, 7K, and 7M). Western blot (FIG. 7A) is representative of two independent experiments. Western blot (FIG. 7L) was performed once with three to four biologically independent tumors. Spheroid experiments were performed twice. Tumor growth, staining and hydroxyproline experiments were performed once with multiple biologically independent tumors.

FIGS. 8A-8O. TGFβ-induced collagen synthesis is linked to glutamine-dependent TCA cycle anaplerosis. FIG. 8A Growth curves of PSCs cultured in the indicated percentage of Gln and treated with TGFβ (2 ng/mL). n=3 biologically independent samples. FIG. 8B Western Blot of PSCs cultured in 100% or 20% Gln and treated with TGFβ for 48 hours. FIG. 8C Collagen abundance in ECM derived from confluent PSCs cultured in the indicated percentage of Gln and treated with TGFβ. n=3 biologically independent samples. FIG. 8D Growth curves of MFBs cultured in the indicated percentage of Gln and treated with TGFβ. n=3 biologically independent samples. FIG. 8E Western Blot of MFBs cultured in 100% or 20% Gln and treated with TGFβ for 48 hours. FIG. 8F Collagen abundance in ECM derived from confluent MFBs cultured in the indicated percentage of Gln and treated with TGFβ. n=3 biologically independent samples. FIG. 8G Relative number of NIH-3T3 cells cultured in 10% Gln and treated with TGFβ and 0.2 mM, 1 mM, or 5 mM of asparagine (Asn) or proline (Pro). n=3 biologically independent samples. FIG. 8H Western Blot of NIH-3T3 cells cultured in 10% Gln and treated with TGFβ and the indicated metabolites and concentrations for 48 hours. FIG. 8I Relative metabolite abundance in NIH-3T3 cells cultured in 10% Gln and treated with TGFβ and dm-Glu (5 mM) or dm-aKG (5 mM) for 48 hours. n=3 biologically independent samples. (FIGS. 8J and 8K) Western Blot of PSCs (FIG. 8J) or MFBs (FIG. 8K) cultured in 100% or 20% Gln and treated with TGFβ for 48 hours. (FIGS. 8L and 8M) tRNA charging in PSCs (FIG. 8L) or MFBs (FIG. 8M) cultured in 20% Gln and treated with TGFβ for 48 hours. n=1 independent experiment. (FIGS. 8N and 80) Western Blot of PSCs (FIG. 8N) or MFBs (FIG. 80) cultured in 20% Gln and treated with TGFβ and dm-Glu or dm-aKG for 48 hours. MFBs were also treated with aspartate (Asp, 20 mM). Mean±SD (FIGS. 8A, 8C, 8D, and 8F) or mean+SD (FIG. 8G) are shown. Dashed lines (FIGS. 8A, 8D, and 8G) represent cell number at day 0. Two-way ANOVA (FIGS. 8A, 8C, 8D, and 8F). Western blots are representative of three (FIGS. 8B, 8E, 8J, and 8K) or two (FIGS. 8H, 8N, and 80) independent experiments. tRNA charging analyses (FIGS. 8L and 8M) are representative of two independent experiments. All other experiments were performed at least twice.

FIGS. 9A-9G. Glutamine de novo synthesis can maintain collagen synthesis and proliferation when glutamine is limiting. FIG. 9A Western Blot of PSCs cultured in 20% Gln in the presence of TGFβ and treated with dm-aKG and MSO. FIG. 9B Collagen abundance of in ECM derived from confluent PSCs cultured in 100% or 10% Gln in the presence of TGFβ and treated with dm-aKG, dm-Glu and MSO. n=3 biologically independent samples. FIG. 9C Relative number of NIH-3T3 cells expressing Ctrl or Glul sgRNA, cultured in 10% Gln and treated with TGFβ alone and dm-aKG or dm-Glu. n=2 (Glul-sg6 dm-Glu), n=3 (all others) biologically independent samples. (FIGS. 9D and 9E) Growth curves of NIH-3T3 cells (FIG. 9D) or PSCs (FIG. 9E) expressing Ctrl or Glul sgRNA, cultured in 100% or 10% Gln/20% Gln. n=3 biologically independent samples. FIG. 9F Western Blot of PSCs expressing Ctrl or Glul sgRNA, cultured in 20% Gln for 48 hours. FIG. 9G Collagen abundance in ECM derived from confluent PSCs expressing Ctrl or Glul sgRNA, cultured in 20% Gln. n=3 biologically independent samples. Mean+SD (FIGS. 9B, 9C, 9G) or mean±SD (FIGS. 9D and 9E) are shown. One-way ANOVA with Holm-Sidak correction (FIG. 9B), one-way ANOVA (FIGS. 9C and 9G), two-sided unpaired t-test (C: Glul-sg6 dm-Glu), two-way ANOVA (FIGS. 9D and 9E). Western blots (FIGS. 9A and 9F) are representative of three independent experiments. All other experiments were performed at least twice.

FIGS. 10A-10L. TGFβ suppresses PC expression and reduces PC activity. (FIGS. 10A-10C) mRNA expression of the indicated genes in NIH-3T3 cells (FIG. 10A), PSCs (FIG. 10B) or MFBs (FIG. 10C) cultured in 100% Gln and treated with TGFβ for 24 hours. n=3 biologically independent samples. FIG. 10D Western Blot of PSCs (left) or MFBs (right) cultured in 100% or 20% Gln and treated with TGFβ for 48 hours. FIG. 10E UCSC genome browser tracks showing putative SMAD2/SMAD3/SMAD4 binding motifs, SMAD4 ChIP-seq peaks in HepG2 cells, the Genehancer promoter element and the PC transcriptional start site (TSS) at the genomic loci of three human PC isoforms. FIG. 10F Pcx expression from RNA-sequencing of quiescent PSCs (qPSC), myofibroblastic CAFs (myCAF) and inflammatory CAFs (iCAFs). Data and p-values are from GSE93313. FIG. 10G mRNA expression of myCAF and iCAF markers in iCAFs and myCAFs (left); Pcx mRNA expression in myCAFs and iCAFs, relative to qPSCs (right). n=3 biologically independent samples. FIG. 10H [U-¹³C]-Glc tracing in PSCs cultured in 100% or 20% Gln and treated with TGFβ for 48 hours. N=3 biologically independent samples. FIG. 10I [U-¹³C]-Glc tracing into indicated amino acid residues of cellular proteins. NIH-3T3 cells were cultured in 10% Gln and treated with TGFβ for 48 hours. n=3 biologically independent samples. (FIGS. 10J and 10K) [3,4-13 C]-Glc tracing in PSCs cultured in 100% or 20% Gln and treated with TGFβ for 48 hours. M+1 labeling (FIG. 10J). PC activity (FIG. 10K). n=3 biologically independent samples. FIG. 10L Western Blot of PSCs expressing empty vector or human PC cDNA, cultured in 20% Gln and treated with TGFβ. Mean+SD (FIGS. 10A-10C and 10G-10K) are shown. Two-sided unpaired t-test (FIGS. 10A-10C, 10G, and 10I), by one-way ANOVA with Holm-Sidak correction (FIGS. 10H, 10J, and 10K). Western blots (FIGS. 10D and 10L) are representative of two independent experiments. (3,4-¹³C)-Glc tracing in PC-ko cells (FIG. 10F) was performed once. All other experiments were performed at least twice.

FIGS. 11A-110. PC is required for collagen synthesis when extracellular glutamine is low. FIG. 11A Western Blot of NIH-3T3 cells expressing Ctrl or PC sgRNA, cultured in 100% Gln for 48 hours. FIG. 11B Western Blot of PSCs expressing Ctrl or PC sgRNA, cultured in 20% Gln for 48 hours. FIG. 11C Western Blot of parental MFBs and MFBs expressing Ctrl or PC sgRNA, cultured in 100% or 20% Gln for 48 hours. FIG. 11D Collagen abundance in ECM derived from confluent PSCs (left) or MFBs (right) expressing Ctrl or PC sgRNA, cultured in 20% Gln. n=3 biologically independent samples. FIG. 11E Western Blot of MFBs expressing Ctrl or PC sgRNA, cultured in 20% Gln and treated with dm-αKG for 48 hours. FIG. 11F [3,4-¹³C]-Glc tracing in NIH-3T3 cells expressing Ctrl or PC sgRNA cultured in 100% or 10% Gln for 48 hours. n=3 biologically independent samples. (FIGS. 11G-11I) Growth curves of NIH-3T3 cells (FIG. 11G), PSCs (FIG. 11H) or MFBs (FIG. 111) expressing Ctrl or PC sgRNA, cultured in 100% or 10%/20% Gln. n=3 biologically independent samples. FIG. 11J tRNA charging in PSCs (left) or MFBs (right) expressing Ctrl or PC sgRNA, cultured in 20% Gln for 48 hours. n=1 independent experiment. FIG. 11K Col1a1 mRNA expression in PSCs (left) or MFBs (right) expressing Ctrl or PC sgRNA, cultured in 20% Gln for 48 hours. n=3 biologically independent samples. (FIGS. 11L and 11M) H3K27ac (FIG. 11L) or H3K27me3 enrichment (FIG. 11M) in NIH-3T3 cells expressing Ctrl or PC sgRNA, cultured in 10% Gln for 48 hours. n=3 (FIG. 11L), n=4 (FIG. 11M) independent experiments. (FIGS. 11N and 11O) Col1a1 mRNA expression in NIH-3T3 cells expressing Ctrl or PC sgRNA, cultured in 10% Gln for 48 hours. Cells were treated with dm-Glu (FIG. 11N) or dm-αKG and MSO (FIG. 11O). n=3 biologically independent samples. Mean SD (FIGS. 11G-11I), mean+SD (FIGS. 11D, 11F, and 11K-11O) are shown. Dashed lines (FIGS. 11G-11I) represent cell number at day 0. Two-way ANOVA (FIGS. 11F-11I), one-way ANOVA (FIGS. 11D and 11K), two-way ANOVA (FIGS. 11L and 11M) analyzing the effects of PC-ko on H3K27ac or H3K27me3 across the analyzed genomic regions, one-way ANOVA with Holm-Sidak correction (FIGS. 11N and 11O). Western blots are representative of two (FIGS. 11A and 11E) or three (FIGS. 11B and 11C) independent experiments. tRNA charging analysis (FIG. 11J) is representative of two independent experiments. All other experiments were performed at least twice.

FIGS. 12A-12L. Fibroblasts take up and use lactate for TCA cycle anaplerosis via PC. (FIG. 12A) [U-¹³C]-Glc and [U-¹³C]-Lac tracing. NIH-3T3 cells were cultured for 48 hours in 100% or 10% Gln in the presence of 10 mM or 1 mM D-glucose with or without 10 mM Na-lactate. M+3 isotopologues are shown in FIG. 6G. G, glucose; L, lactate. n=3 biologically independent samples. (FIG. 12B) [U-¹³C]-Lac tracing. NIH-3T3 cells were cultured in 10% Gln and treated with AZD3965 (MCT1 inhibitor, 5 μM) or sodium oxamate (LDH inhibitor, 10 mM) for 8 hours. [U-¹³C]-Lac was added in the last 1 hour. n=2 (LDHi), n=3 (all others) biologically independent samples. (FIGS. 12C and 12D) [1-¹³C]-Lac tracing in NIH-3T3 cells expressing Ctrl or PC sgRNA cultured in 10% Gln in the presence of 10 mM Na-lactate for 48 hours. M+1 labeling (FIG. 12C). PC activity (FIG. 12D). n=3 biologically independent samples. (FIGS. 12E and 12F) Collagen abundance in ECM generated by confluent parental (FIG. 12E) or Ctrl or PC sgRNA expressing NIH-3T3 cells (FIG. 12F) cultured in 10% Gln and the indicated concentrations of D-glucose and Na-lactate. n=3 biologically independent samples. (FIG. 12G) Western Blot of PSCs cultured in 20% Gln and the indicated concentrations of D-glucose for 48 hours. (FIG. 12H) [U-¹³C]-Glc and [U-¹³C]-Lac tracing into indicated metabolites. PSCs were cultured for 48 hours in 20% Gln and 1 mM D-glucose with or without 10 mM Na-lactate. n=3 biologically independent samples. (FIG. 121) [U-¹³C]-Lac tracing in PSCs cultured in 100% or 20% Gln for 48 hours. n=3 biologically independent samples. (FIG. 12J) [1-¹³C]-Lac tracing. PSCs were cultured for 48 hours in 100% or 20% Gln in the presence of 10 mM or 1 mM D-glucose and 10 mM Na-lactate. n=3 biologically independent samples. (FIG. 12K) Western Blot of PSCs cultured in 20% Gln and the indicated concentrations of D-glucose and Na-lactate for 48 hours. (FIG. 12L) Western Blot of PSCs expressing Ctrl or PC sgRNA, cultured in 20% Gln and the indicated concentrations of D-glucose and Na-lactate for 48 hours. Mean+SD (FIGS. 12A-12F and 12H-12J) are shown. Two-sided unpaired t-test (FIG. 12B), one-way ANOVA (FIGS. 12C, 12D, and 12J), one-way ANOVA with Holm-Sidak correction (FIGS. 12E and 12F), two-sided unpaired t-test with Holm-Sidak correction (FIG. 12I). Western blots (FIGS. 12G, 12K, and 12L) are representative of two independent experiments. [U-¹³C]-Lac tracing in PSC in low glucose (FIG. 12H) was performed once. All other experiments were performed at least twice.

FIGS. 13A-13M. Fibroblast PC supports tumor fibrosis and growth. FIG. 13A Outgrowth of KPC spheroids on top of a synthetic ECM (3D gel) Representative images are shown. Scale bar=500 μm. FIG. 13B Pearson correlation of total spheroid area from (FIG. 13A) with collagen 1 concentration used to prepare the synthetic ECM. n=4 biologically independent samples. (FIGS. 13C and 13D) Western blot of ECM generated by confluent PSCs cultured in 100% or 10% Gln in the presence of TGFβ (FIG. 13C). (FIG. 13D) Outgrowth of KPC spheroids on top of this ECM. Representative images are shown. Scale bar=500 μm. FIG. 13E Quantification of spheroid outgrowth from (FIG. 13D). n=8 biologically independent samples. FIG. 13F Survival of KPC-GFP cells after 3 days co-culture with Ctrl or Glul sgRNA expressing PSCs on plastic or PSC-derived ECM in the absence of FBS and Gln. n=3 biologically independent samples. (FIGS. 13G-13K) KPC/PSC allograft experiment in nude mice. (FIG. 13G) Representative images of Picrosirius staining of KPC/PSC allografts at day 25 after injection. Scale bar-500 μm. (FIG. 13H) Quantification of Picrosirius staining of KPC/PSC allografts. n=8 biologically independent tumors. (FIG. 13I) Collagen levels in KPC/PSC allografts at day 25 after injection. n=5 biologically independent tumors. (FIG. 13J) Representative image of KPC/PSC allograft tumors stained for αSMA, CK8 and DAPI. Scale bar=100 μm. (FIG. 13K) Western Blot of KPC/PSC allografts at day 25 after injection. FIG. 13L Volume of DB7 allografts 8 days after injection of DB7 cells alone, with Matrigel or with MFBs. n=4 (DB7+Matrigel), n=8 (DB7, DB7+MFB) biologically independent tumors. FIG. 13M Western Blot of the second batch of DB7/MFB allografts 8 days after injection. The first batch is shown in FIG. 7L. Mean±SD (FIGS. 13B and 13E), median with 25% to 75% percentile box and min/max whiskers (FIGS. 13H, 13I, and 13L), mean+SD (FIG. 13F) are shown. Pearson correlation followed by two-sided unpaired t-test (FIG. 13B), two-way ANOVA (FIG. 13E), two-way ANOVA with Holm-Sidak correction (FIG. 13F), one-way ANOVA (FIG. 13H), two-sided unpaired t-test (FIG. 13I), one-way ANOVA with Holm-Sidak correction (FIG. 13L). Western blots were performed once with 5 (FIG. 13K) or 3-4 (FIG. 13M) biologically independent tumors, or were performed twice (FIG. 13C). Spheroid experiments were performed twice. Tumor growth, staining and hydroxyproline experiments were performed once with multiple biologically independent tumors.

FIGS. 14A-14B. Gating strategy for flow cytometry. FIG. 14A Gating strategy for OPP staining. Data are from FIG. 1F. FIG. 14B Gating strategy for KPC-GFP and PSC co-culture assay. Data are from FIG. 13F.

### DETAILED DESCRIPTION

### Methods of Treatment

In some aspects, methods provided in the present disclosure are drawn to treating a disease or disorder by administering to a subject in need thereof an antagonist of pyruvate carboxylase and an antagonist of glutaminase.

### Pyruvate Carboxylase (PC)

In some aspects, methods and compositions provided in the present disclosure comprise an antagonist of pyruvate carboxylase (PC). PC is a mitochondrial enzyme that catalyzes the carboxylation of pyruvate to form oxalacetate in the tricarboxylic acid (TCA) cycle, gluconeogenesis, adipogenesis, and glucose homeostasis in pancreatic islet cells and astrocytes. As demonstrated herein, oxaloacetate is required for cell proliferation and extracellular matrix protein production. Thus, antagonizing the activity of PC (e.g., with a PC antagonist) is an effective strategy for inhibiting cell proliferation and extracellular matrix protein production.

PC herein may be PC expressed by any organism known in the art. PC is conserved in human (Gene ID: 5091), rat (Gene ID: 25104), mouse (Gene ID: 18563), cow (Gene ID: 338471), non-human primates (Gene IDs: 71303, 116469064, 108518689), dog (Gene ID: 483704), and zebrafish (Gene ID: 58068). In some embodiments, PC is human PC.

Human PC may be any human PC. Human PC is alternatively expressed as 3 different mRNA sequences. The human PC protein translated from the 3 different mRNA sequences is the same and is an 1,178 amino acid protein. In some embodiments, human PC is expressed as mRNA sequence 1 (NM_000920.4). In some embodiments, human PC is expressed as mRNA sequence 2 (NM_001040716.2). In some embodiments, human PC is expressed as mRNA sequence 3 (NM_002172.3).

In some embodiments, an antagonist of PC is administered to a subject in need thereof. An antagonist is a compound or molecule that inhibits the activity of a protein. An antagonist of PC may decrease PC activity by 10%-100%, 20%-90%, 30%-80%, 40%-70%, or 50%-60%. In some embodiments, an antagonist of PC may decrease PC activity by 10%, 15%, 20%, 25%, 30%, 35%, 40%, 45%, 50%, 55%, 60%, 65%, 70%, 75%, 80%, 85%, 90%, 95%, 100%, or more. In some embodiments, PC activity is decreased as measured by any suitable assay, including but not limited to, an immunoassay, a hybridization-based assay, or a sequencing-based assay (e.g., RNA-Seq).

An antagonist of PC inhibits the activity of PC directly or indirectly. A direct antagonist of PC binds to PC protein and inhibits its catalytic activity (e.g., by blocking the enzyme active site). An indirect antagonist of PC inhibits the production of PC protein (e.g., PC transcription, PC translation).

An antagonist of PC may be any PC antagonist known in the art. Non-limiting examples of potential PC antagonists include small molecules having a molecular weight of less than about 1,000 g/mol; nucleic acids compounds including a guide RNA (gRNA) used in a clustered regularly interspaced short palindromic repeats (CRISPR/Cas) genome editing system, an antisense oligonucleotide, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), a short (or small) activating RNA (saRNA), or a combination thereof; a protein (e.g., anti-PC antibody); a polypeptide (e.g., containing the PC active site); and an anti-PC nucleic acid aptamer.

In some embodiments, an antagonist of PC is an antagonist known in the art including, but not limited to: ((N4-((5-(4-(benzyloxy)phenyl)-2-thiophenyl)methyl-N2-isobutyl-2,4,-pyrimidinediamine); Phenylacetic acid (PAA); chlorothricin; sodium benzoate; Phenylacetate; Phenylacetyl CoA; valeryl CoA; n-decanoyl CoA; CHEBI:90318; 2-hydroxy-3-(quinolone-2-yl)propenoic acid; oxamate; 3-hydroxypyruvate; 3-bromopyruvate; hydroxyamic acid; a-hydroxycinnamic acid; phosphonoacetate; or acetyl coenzyme A trisodium. In some embodiments, an antagonist of PC is described in US Publication No. 2011/0158980 or WO Publication No. 2012/174470A1, both of which are incorporated by reference herein.

In some embodiments, an antagonist of PC is a guide RNA (gRNA) used in a CRISPR/Cas genome editing system. CRISPR/Cas genome editing is well-known in the art. (see, e.g., Wang et al., Ann. Rev. Biochem., 2016, 85: 227-264; Pickar-Oliver and Gersbach, Nature Reviews Molecular Cellular Biology, 2019, 20: 490-507; Aldi, Nature Communications, 2018, 9: 1911). In some embodiments, a gRNA antagonist of PC knocks out (removes) PC from the genome, decreases expression of PC from the genome, decreases PC enzyme activity, or a combination thereof. A gRNA antagonist of PC may be 1-10, 2-9, 3-8, 4-7, or 5-6 gRNAs. In some embodiments, a gRNA antagonist of PC may be 1, 2, 3, 4, 5, 6, 7, 8, 9, or 10 or more gRNAs.

A subject in need thereof may be administered one antagonist of PC or multiple antagonists of PC. When multiple antagonists of PC are administered, the multiple antagonists may have the same mechanism of action (e.g., inhibiting PC expression, inhibiting PC enzymatic activity), different mechanisms of action, or a combination thereof.

In some embodiments, 1-10, 2-9, 3-8, 4-7, or 5-6 antagonists of PC are administered to a subject in need thereof. In some embodiments, 1, 2, 3, 4, 5, 6, 7, 8, 9, or 10 or more antagonists of PC are administered to a subject in need thereof. When multiple antagonists of PC are administered to a subject, they may be administered in the same administration or in multiple administrations.

### Glutaminase

In some aspects, methods and compositions provided herein comprise an antagonist of glutaminase. Glutaminase is an amidohydrolase enzyme that generates glutamate from glutamine. Glutaminase has tissue-specific isoenzymes, including “kidney-type” (GLS1) and “liver-type” (GLS2). Glutaminase is expressed in numerous tissues, including, but not limited to, liver, epithelial cells, kidney, small intestine, large intestine, and central nervous system.

Glutaminase may be glutaminase expressed in any organism known in the art. Glutaminase is conserved in human (Gene ID: 2744), mouse (Gene ID: 14660), rat (Gene ID: 24398), zebrafish (Gene ID: 564147, 564746, 556445), pig (Gene ID: 399525), frog (Gene ID: 100379734), cow (Gene ID: 525335), dog (Gene ID: 488448), non-human primate (Gene ID: 693520, 470606, 101926081). In some embodiments, glutaminase is human glutaminase.

Human glutaminase may be any human glutaminase sequence known in the art. Human glutaminase is alternatively expressed as 2 different mRNA sequences. The human glutaminase protein translated from the 2 different mRNA sequences is the same and is a 174 amino acid protein. In some embodiments, human glutaminase is expressed as mRNA sequence 1 (NM_001256310.2). In some embodiments human glutaminase is expressed as mRNA sequence 2 (NM_014905.5).

In some embodiments, an antagonist of glutaminase is administered to a subject in need thereof. An antagonist is a compound or molecule that inhibits the activity of a protein. An antagonist of glutaminase may decrease glutaminase activity by 10%-100%, 20%-90%, 30%-80%, 40%-70%, or 50%-60%. In some embodiments, an antagonist of glutaminase may decrease glutaminase activity by 10%, 15%, 20%, 25%, 30%, 35%, 40%, 45%, 50%, 55%, 60%, 65%, 70%, 75%, 80%, 85%, 90%, 95%, 100%, or more. In some embodiments, glutaminase activity is decreased as measured by any suitable assay, including but not limited to, an immunoassay, a hybridization-based assay, or a sequencing-based assay (e.g., RNA-Seq).

An antagonist of glutaminase inhibits the activity of glutaminase directly or indirectly. A direct antagonist of glutaminase binds to glutaminase protein and inhibits its catalytic activity (e.g., by blocking the enzyme active site). An indirect antagonist of glutaminase inhibits the production of glutaminase protein (e.g., GLS transcription, glutaminase translation).

An antagonist of glutaminase may be any glutaminase antagonist known in the art. Non-limiting examples of potential glutaminase antagonists include small molecules having a molecular weight of less than about 1,000 g/mol; nucleic acids compounds include a guide RNA (gRNA) used in a clustered regularly interspaced short palindromic repeats (CRISPR/Cas) genome editing system, an antisense oligonucleotide, a ribozyme, a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), a short (or small) activating RNA (saRNA), or a combination thereof; a protein (e.g., an anti-glutaminase antibody); a polypeptide (e.g., including glutaminase active site); and an anti-glutaminase nucleic acid aptamer.

In some embodiments, an antagonist of glutaminase is an antagonist known in the art including, but not limited to: CB-839; Bis-2-(5-phenylacetamido-1,3,4-thiadiazol-2-yl) ethyl sulphide (BTPES); V-9302; benzophenanthridinone (compound 968); Rais-5C; Nedelcovych-13d; DRP-104; UPGL00004; GK921; JHU395; 6-diazo-5-oxo-L-norleucine (DON); acivicin; azaserine; L-y-Glutamyl-p-nitoanilide (GPNA); Shukla 11b; IACS-6274; THDP-17; JHU-083; (S)-2-hydroxy-2-phenyl-N-(5-(4-(6-(2-(3-(trifluoromethoxy)phenyl)aceta-mido)pyridazin-3-yl)butyl)-1,3,4-thiadiazol-2-yl)acetamide; N-methyl-1-14-[6-(2-{4-[3-(trifluoromethoxy)phenyl]pyridin-2-yl}acetamido-)pyridazin-3-yl]butyl}-1H-1,2,3-triazole-4-carboxamide; 1-(2-fluoro-4-(5-(2-(pyridin-2-yl)acetamido)-1,3,4-thiadiazol-2-yl)butyl)--N-((4-(trifluoromethyl)pyridin-2-yl)methyl)-1H-1,2,3-triazole-4-carboxami-de; 1-(2-fluoro-4-(6-(2-(4-(trifluoromethyl)pyridin-2-yl)acetamido)pyri-dazin-3-yl)butyl)-N-methyl-1H-1,2,3-triazole-4-carboxamide; N-(pyridin-2-ylmethyl)-5-(3-(6-(2-(3-(trifluoromethoxy)phenyl)acetamido)p-yridazin-3-yl)pyrrolidin-1-yl)-1,3,4-thiadiazole-2-carboxamide; (R)-1-(2-fluoro-4-(6-(2-(4-(3-(trifluoromethoxy)phenyl)pyridin-2-yl)aceta-mido)-pyridazin-3-yl)butyl)-N-methyl-1H-1,2,3-triazole-4-carboxamide; (R)-1-(2-fluoro-4-(6-(2-(4-(trifluoromethyl)pyridin-2-yl)acetamido)pyrida-zin-3-yl)butyl)-N-methyl-1H-1,2,3-triazole-4-carboxamide; (R)-1-(2-fluoro-4-(6-(2-(6-methyl-4-(trifluoromethyl)pyridin-2-yl)acetami-do)pyridazin-3-yl)butyl)-N-methyl-1H-1,2,3-triazole-4-carboxamide; (R)-1-(4-(6-(2-(4-(cyclopropyldifluoromethyl)pyridin-2-yl)acetamido)pyrid-azin-3-yl)-2-fluorobutyl)-N-methyl-1H-1,2,3-triazole-4-carboxamide; (R)-1-(4-(6-(2-(4-(3,3-difluorocyclobutoxy)pyridin-2-yl)acetamido)pyridaz-in-3-yl)-2-fluorobutyl)-N-methyl-1H-1,2,3-triazole-4-carboxamide; (R)-1-(2-fluoro-4-(6-(2-(1-(3-(trifluoromethoxy)phenyl)-1H-imidazol-4-yl)-acetamido)pyridazin-3-yl)butyl)-N-methyl-1H-1,2,3-triazole-4-carboxamide; 1-(4-(6-(2-(4-cyclobutoxypyridin-2-yl)acetamido)pyridazin-3-yl)butyl)--N-methyl-1H-1,2,3-triazole-4-carboxamide; 1-(4-(6-(2-(4-cyclobutoxypyridin-2-yl)acetamido)pyridazin-3-yl)-2-fluorob-utyl)-N-methyl-1H-1,2,3-triazole-4-carboxamide; 1-(4-(6-(2-(4-(3,3-difluorocyclobutoxy)pyridin-2-yl)acetamido)pyridazin-3-yl)butyl)-N-methyl-1H-1,2,3-triazole-4-carboxamide; 1-(4-(6-(2-(4-(3,3-difluorocyclobutoxy)pyridin-2-yl)acetamido)pyridazin-3-yl)-2-fluorobutyl)-N-methyl-1H-1,2,3-triazole-4-carboxamide; (R)-1-(4-(6-(2-(4-cyclopropylpyridin-2-yl)acetamido)pyridazin-3-yl)-2-flu-orobutyl)-N-methyl-1H-1,2,3-triazole-4-carboxamide; 5-(3-(6-(2-(pyridin-2-yl)acetamido)pyridazin-3-yl)pyrrolidin-1-yl)-N-((4--(trifluoromethyl)pyridin-2-yl)methyl)-1,3,4-thiadiazole-2-carboxamide; N,N′-(5,5′-(cyclohexane-1,3-diyl)bis(1,3,4-thiadiazole-5,2-diyl))bis(2-phenylacetamide) (both or either of 1S,3S and 1R,2R enantiomers); 2-Phenyl-N-{5-[1-(5-phenylacetylamino-[1,3,4]thiadiazol-2-yl)-piperidin-4--yloxy]-[1,3,4]thiadiazol-2-yl}-acetamide; N-{5-[1-(5-Acetylamino-[1,3,4]thiadiazol-2-yl)-piperidin-4-yloxy]-[1,3,4]-thiadiazol-2-yl}-acetamide; N-{5-[1-(5-Amino-[1,3,4]thiadiazol-2-yl)-piperidin-4-yloxy]-[1,3,4]thiadi-azol-2-yl}-2-phenyl-acetamide; 2-(Pyridin-3-yl)-N-(5-(4-((5-(2-(pyridin-3-yl)acetamido)-1,3,4-thiadiazol--2-yl)oxy)piperidin-1-yl)-1,3,4-thiadiazol-2-yl)acetamido; 2-Cyclopropyl-N-(5-(4-((5-(2-cyclopropylacetamido)-1,3,4-thiadiazol-2-yl)-oxy)piperidin-1-yl)-1,3,4-thiadiazol-2-yl)acetamido; 2-Phenyl-N-{6-[1-(6-phenylacetylamino-pyridazin-3-yl)-piperidin-4-yloxy]--pyridazin-3-yl}-acetamide; 2-Phenyl-N-(5-(4-((5-(2-phenylacetamido)-1,3,4-thiadiazol-2-yl)amino)pipe-ridin-1-yl)-1,3,4-thiadiazol-2-yl)acetamido; 2-Phenyl-N-{6-[1-(5-phenylacetylamino-[1,3,4]thiadiazol-2-yl)-piperidin-4--yloxy]-pyridazin-3-yl}-acetamide; N-(6-{1-[5-(2-Pyridin-2-yl-acetylamino)-[1,3,4]thiadiazol-2-yl]-piperidin--4-yloxy}-pyridazin-3-yl)-2-(3-trifluoromethoxy-phenyl)-acetamide; 2-Phenyl-N-{5-[1-(5-phenylacetylamino-[1,3,4]thiadiazol-2-yl)-piperidin-4--ylmethoxy]-[1,3,4]thiadiazol-2-yl}-acetamide; 2-(Pyridin-2-yl)-N-15-[(1-{5-[2-(pyridin-2-yl)acetamido]-1,3,4-thiadiazol--2-yl]piperidin-4-yl)amino]-1,3,4-thiadiazol-2-yl]acetamido; 2-(Pyridin-3-yl)-N-{5-[(1-15-[2-(pyridin-3-yl)acetamido]-1,3,4-thiadiazol--2-yl}piperidin-4-yl)amino]-1,3,4-thiadiazol-2-yl}acetamido; 2-(Pyridin-2-yl)-N-{5-[(1-15-[2-(pyridin-2-yl)acetamido]-1,3,4-thiadiazol--2-yl}piperidin-4-yl)oxy]-1,3,4-thiadiazol-2-yl}acetamido; 2-(Pyridin-4-yl)-N-{5-[(1-15-[2-(pyridin-4-yl)acetamido]-1,3,4-thiadiazol--2-yl}piperidin-4-yl)amino]-1,3,4-thiadiazol-2-yl}acetamido; or 2-Cyclopropyl-N-[5-(4-1[5-(2-phenylacetamido)-1,3,4-thiadiazol-2-yl]amino-}piperidin-1-yl)-1,3,4-thiadiazol-2-yl]acetamido; (R)-1-(4-(6-(2-(4-(3,3-difluorocyclobutoxy)-6-methylpyridin-2-yl)acetamid-o)pyridazin-3-yl)-2-fluorobutyl)-N-methyl-1H-1,2,3-triazole-4-carboxamide; SU-1; SU-2; SU-3; SU-4; SU-5; SU-6; SU-7; SU-8; SU-9; SU-10; SU-11; SU-12; SU-13; SU-14; SU-15; SU-16; SU-17; SU-18; SU-19; SU-20; SU-21; SU-22; SU-23; SU-24; SU-25; SU-26; SU-27; SU-28; SU-29; SU-30; SU-31; SU-32; SU-33; SU-34; SU-35; SU-36; 2-(Pyridin-2-yl)-N-(5-(1-(6-(2-(3-(trifluoromethoxy)phenyl)acetamido)pyrid-in-3-yl)piperidin-4-yl)-1,3,4-thiadiazol-2-yl)acetamide; (RS)-2-(Pyridin-2-yl)-N-(5-(1-(6-(2-(3-(trifluoromethoxy)phenyl)acetamido)-pyridin-3-yl)piperidin-3-yl)-1,3,4-thiadiazol-2-yl)acetamide; (R) or (S) 2-(Pyridin-2-yl)-N-(5-(1-(6-(2-(3-(trifluoromethoxy)phenyl)acet-amido) pyridin-3-yl)piperidin-3-yl)-1,3,4-thiadiazol-2-yl)acetamide; (S) or (R) 2-(Pyridin-2-yl)-N-(5-(1-(6-(2-(3-(trifluoromethoxy)phenyl)acet-amido) pyridin-3-yl)piperidin-3-yl)-1,3,4-thiadiazol-2-yl)acetamide; (RS)-2-(Pyridin-3-yl)-N-(5-(1-(6-(2-(3-(trifluoromethoxy)phenyl)acetamido)-pyridin-3-yl) piperidin-3-yl)-1,3,4-thiadiazol-2-yl)acetamide; (R) or (S) 2-(Pyridin-3-yl)-N-(5-(1-(6-(2-(3-(trifluoromethoxy)phenyl)acet-amido) pyridin-3-yl)piperidin-3-yl)-1,3,4-thiadiazol-2-yl)acetamide; (S) or (R) 2-(Pyridin-3-yl)-N-(5-(1-(6-(2-(3-(trifluoromethoxy)phenyl)acet-amido) pyridine-3-yl)piperidin-3-yl)-1,3,4-thiadiazol-2-yl)acetamide; 2-(Pyridin-3-yl)-N-(5-(1-(6-(2-(3-(trifluoromethoxy)phenyl)acetamido)pyrid-in-3-yl)piperidin-4-yl)-1,3,4-thiadiazol-2-yl)acetamide; 2-(3-Cyanophenyl)-N-(5-(1-(6-(2-(3-(trifluoromethoxy)phenyl)acetamido)pyri-din-3-yl)piperidin-4-yl)-1,3,4-thiadiazol-2-yl)acetamide; 2-(Pyridin-2-yl)-N-(5-(4-(5-(2-(3-(trifluoromethoxy)phenyl)acetamido)-1,3,-4-thiadiazol-2-yl)piperidin-1-yl)pyridin-2-yl)acetamide; 2-(Pyridin-2-yl)-N-(5-(3-(5-(2-(3-(trifluoromethoxy)phenyl)acetamido)-1,3,-4-thiadiazol-2-yl)piperidin-1-yl)pyridin-2-yl)acetamide; 2-(pyridin-2-yl)-N-(5-(1-(6-(2-(3-(trifluoromethoxy)phenyl)acetamido)pyrid-azin-3-yl)piperidin-4-yl)-1,3,4-thiadiazol-2-yl)acetamide; 2-(Pyridin-2-yl)-N-(5-(1-(6-(2-(3-(trifluoromethoxy)phenyl)acetamido)pyrid-azin-3-yl)piperidin-3-yl)-1,3,4-thiadiazol-2-yl)acetamide; 2-(Pyridin-3-yl)-N-(5-(1-(6-(2-(3-(trifluoromethoxy)phenyl)acetamido)pyrid-azin-3-yl)piperidin-4-yl)-1,3,4-thiadiazol-2-yl)acetamide; 2-(3-(Methylsulfonamido)phenyl)-N-(5-(1-(6-(2-(3-(trifluoromethoxy)phenyl)-acetamido) pyridazin-3-yl)piperidin-4-yl)-1,3,4-thiadiazol-2-yl)acetamide; 2-(2-Chlorophenyl)-N-(6-(4-(5-(2-(pyridin-2-yl)acetamido)-1,3,4-thiadiazol--2-yl) piperidin-1-yl)pyridazin-3-yl)acetamide; 2-(2-Chlorophenyl)-N-(5-(1-(6-(2-(3-(trifluoromethoxy)phenyl)acetamido)pyr-idazin-3-yl)piperidin-3-yl)-1,3,4-thiadiazol-2-yl)acetamide; 2-(2-Fluorophenyl)-N-(6-(4-(5-(2-(pyridin-2-yl)acetamido)-1,3,4-thiadiazol--2-yl)piperidin-1-yl)pyridazin-3-yl)acetamide; 2-(Pyrazin-2-yl)-N-(5-(1-(6-(2-(3-(trifluoromethoxy)phenyl)acetamido)pyrid-azin-3-yl)piperidin-4-yl)-1,3,4-thiadiazol-2-yl)acetamide; 2-(Pyridin-2-yl)-N-(5-(1-(6-(2-(3-(trifluoromethoxy)phenyl)acetamido)pyrid-azin-3-yl)piperidin-4-yl)-1,3,4-thiadiazol-2-yl)acetamide Dihydrochloride; 2-(Pyridin-2-yl)-N-(6-(4-(5-(2-(3-(trifluoromethoxy)phenyl)acetamido)-1,3,-4-thiadiazol-2-yl)piperidin-1-yl)pyridazin-3-yl)acetamide; 2-(Pyridin-3-yl)-N-(6-(4-(5-(2-(3-(trifluoromethoxy)phenyl)acetamido)-1,3,-4-thiadiazol-2-yl)piperidin-1-yl)pyridazin-3-yl)acetamide; 2-(Pyridin-3-yl)-N-(6-(4-(5-(2-(2,3,6-trifluorophenyl)acetamido)-1,3,4-thi-adiazol-2-yl)piperidin-1-yl)pyridazin-3-yl)acetamide; 2-(Pyridin-2-yl)-N-(6-(4-(5-(2-(2,3,6-trifluorophenyl)acetamido)-1,3,4-thi-adiazol-2-yl)piperidin-1-yl)pyridazin-3-yl)acetamide; 2-(2,3-Difluorophenyl)-N-(5-(1-(6-(2-(pyridin-2-yl)acetamido)pyridazin-3-y-1)piperidin-4-yl)-1,3,4-thiadiazol-2-yl)acetamide; 2-(3,4-Difluorophenyl)-N-(5-(1-(6-(2-(pyridin-2-yl)acetamido)pyridazin-3-y-l)piperidin-4-yl)-1,3,4-thiadiazol-2-yl)acetamide; 2-(2-Fluorophenyl)-N-(5-(1-(6-(2-(pyridin-2-yl)acetamido)pyridazin-3-yl)pi-peridin-4-yl)-1,3,4-thiadiazol-2-yl)acetamide; 2-(3-Fluorophenyl)-N-(5-(1-(6-(2-(pyridin-2-yl)acetamido)pyridazin-3-yl)pi-peridin-4-yl)-1,3,4-thiadiazol-2-yl)acetamide; 2-(4-Fluorophenyl)-N-(5-(1-(6-(2-(pyridin-2-yl)acetamido)pyridazin-3-yl)pi-peridin-4-yl)-1,3,4-thiadiazol-2-yl)acetamide; 2-(2-Methoxyphenyl)-N-(5-(1-(6-(2-(pyridin-2-yl)acetamido)pyridazin-3-yl)p-iperidin-4-yl)-1,3,4-thiadiazol-2-yl)acetamide; 2-(2-Chlorophenyl)-N-(5-(1-(6-(2-(pyridin-2-yl)acetamido)pyridazin-3-yl)pi-peridin-4-yl)-1,3,4-thiadiazol-2-yl)acetamide; 2-(5-Chloro-2-(trifluoromethyl)phenyl)-N-(5-(1-(6-(2-(pyridin-2-yl)acetami-do) pyridazin-3-yl)piperidin-4-yl)-1,3,4-thiadiazol-2-yl)acetamide; 2-(4-Chlorophenyl)-N-(5-(1-(6-(2-(pyridin-2-yl)acetamido)pyridazin-3-yl)pi-peridin-4-yl)-1,3,4-thiadiazol-2-yl)acetamide; 2-(Quinolin-6-yl)-N-(5-(1-(6-(2-(3-(trifluoromethoxy)phenyl)acetamido)pyri-dazin-3-yl)piperidin-4-yl)-1,3,4-thiadiazol-2-yl)acetamide; 2-o-Tolyl-N-(5-(1-(6-(2-(3-(trifluoromethoxy)phenyl)acetamido)pyridazin-3--yl)piperidin-4-yl)-1,3,4-thiadiazol-2-yl)acetamide; N-(6-(4-(5-(2-(1H-indol-3-yl)acetamido)-1,3,4-thiadiazol-2-yl)piperidin-1--yl)pyridazin-3-yl)-2-(3-(trifluoromethoxy)phenyl)acetamide; 2-(2-Fluorophenyl)-N-(6-(4-(5-(2-(pyrazin-2-yl)acetamido)-1,3,4-thiadiazol--2-yl)piperidin-1-yl)pyridazin-3-yl)acetamide; 2-(3-(Azetidin-1-yl)phenyl)-N-(5-(1-(6-(2-(3-(trifluoromethoxy)phenyl)acet-amido)pyridazin-3-yl)piperidin-4-yl)-1,3,4-thiadiazol-2-yl)acetamide; 2-(3-Chlorophenyl)-N-(5-(1-(6-(2-(pyridin-2-yl)acetamido)pyridazin-3-yl)pi-peridin-4-yl)-1,3,4-thiadiazol-2-yl)acetamide; 3-Hydroxy-2-phenyl-N-(5-(1-(6-(2-(3-(trifluoromethoxy)phenyl)acetamido)pyr-idazin-3-yl)piperidin-4-yl)-1,3,4-thiadiazol-2-yl)propenamide; (R)-2-hydroxy-2-phenyl-N-(5-(1-(6-(2-(3-(trifluoromethoxy)phenyl)acetamido-)pyridazin-3-yl)piperidin-4-yl)-1,3,4-thiadiazol-2-yl)acetamide; 2-(3-(3-Fluoroazetidin-1-yl)phenyl)-N-(5-(1-(6-(2-(3-(trifluoromethoxy)phe-nyl)acetamido)pyridazin-3-yl)piperidin-4-yl)-1,3,4-thiadiazol-2-yl)acetami-de; or 2-(Pyridin-2-yl)-N-(5-((1-(6-(2-(3-(trifluoromethoxy)phenyl)acetamido)pyri-dazin-3-yl)piperidin-4-yl)methyl)-1,3,4-thiadiazol-2-yl)acetamide. In some embodiments, an antagonist of GLS is disclosed in WO Publication No. 2016/014890; U.S. Pat. No. 11,046,945; U.S. Pat. No. 11,0045,443; U.S. Pat. Nos. 11,013,724; 10,954,257; 10,899,740; 10,842,763; 10,793,535; 10,786,471; 10,766,892; 10,783,066; 10,722,487; 10,660,861; 10,611,759; 10,441,587; 10,344,025; 10,336,778; 10,245,254; 10,125,128; 9,938,267; 9,809,588; 9,783,533; 8,865,718; 5,552,455; or US Publication No. 2017/0209387, each of which is incorporated herein.

In some embodiments, an antagonist of glutaminase is a guide RNA (gRNA) used in a CRISPR/Cas genome editing system. CRISPR/Cas genome editing is well-known in the art. (see, e.g., Wang et al., Ann. Rev. Biochem., 2016, 85: 227-264; Pickar-Oliver and Gersbach, Nature Reviews Molecular Cellular Biology, 2019, 20: 490-507; Aldi, Nature Communications, 2018, 9: 1911). In some embodiments, a gRNA antagonist of glutaminase knocks out (removes) GLS from the genome, decreases expression of GLS from the genome, decreases glutaminase enzyme activity, or a combination thereof. A gRNA antagonist of glutaminase may be 1-10, 2-9, 3-8, 4-7, or 5-6 gRNAs. In some embodiments, a gRNA antagonist of glutaminase may be 1, 2, 3, 4, 5, 6, 7, 8, 9, or 10 or more gRNAs.

A subject in need thereof may be administered one antagonist of glutaminase or multiple antagonists of glutaminase. When multiple antagonists of glutaminase are administered, the multiple antagonists may have the same mechanism of action (e.g., inhibiting glutaminase expression, inhibiting glutaminase enzymatic activity), different mechanisms of action, or a combination thereof. In some embodiments, 1-10, 2-9, 3-8, 4-7, or 5-6 antagonists of glutaminase are administered to a subject in need thereof. In some embodiments, 1, 2, 3, 4, 5, 6, 7, 8, 9, or 10 or more antagonists of glutaminase are administered to a subject in need thereof. When multiple antagonists of glutaminase are administered to a subject, they may be administered in the same administration or in multiple administrations.

A subject in need thereof may be administered one antagonist of PC and one antagonist of glutaminase or multiple antagonists of PC and glutaminase. When one or more antagonist of PC and one or more antagonist of glutaminase are administered, the one or more PC antagonists and one or more glutaminase antagonists may have the same mechanism of action (e.g., inhibiting PC or glutaminase expression, inhibiting PC or glutaminase enzymatic activity), different mechanisms of action, or a combination thereof. In some embodiments, 1-10, 2-9, 3-8, 4-7, or 5-6 antagonists of PC and 1-10, 2-9, 3-8, 4-7, or 5-6 antagonists of glutaminase are administered to a subject in need thereof. In some embodiments, 1, 2, 3, 4, 5, 6, 7, 8, 9, or 10 or more antagonists of PC and 1, 2, 3, 4, 5, 6, 7, 8, 9, or 10 or more antagonists of glutaminase are administered to a subject in need thereof.

When one or more antagonists of PC and one or more antagonists of glutaminase are administered to a subject, they may be administered in the same administration or in multiple administrations.

### Fibrotic Disorder

In some aspects, the present disclosure provides a method of treating a fibrotic disorder by administering to a subject in need thereof an antagonist of PC, an antagonist of glutaminase, or an antagonist of PC and an antagonist of glutaminase in an amount effective to treat the fibrotic disorder. A fibrotic disorder is a disorder in which extracellular matrix molecules uncontrollably and progressively accumulate in affected tissues and organs, causing their ultimate failure. Fibrosis is a predominant feature of the pathology of a wide range of diseases across numerous organ systems, and fibrotic disorders are estimated to contribute to up to 45% of all-cause mortality in the United States. Despite this prevalence of fibrotic disorders, effective therapies are limited.

In some embodiments, a fibrotic disorder that is treated with a method provided herein is characterized by hyperproduction of an extracellular matrix (ECM) protein.

Hyperproduction of an ECM protein is production of an ECM protein that is increased compared to a cell that is not fibrotic or subject that does not have fibrotic disorder. Hyperproduction may be ECM protein production that is increased 5%-100%, 10%-95%, 20%-90%, 30%-80%, 40%-70%, or 50%-60%. In some embodiments, hyperproduction of an ECM is production that is increased 5%, 10%, 15%, 20%, 25%, 30%, 35%, 40%, 45%, 50%, 55%, 60%, 65%, 70%, 75%, 80%, 85%, 90%, 95%, 100% or more.

An ECM protein is a protein in a three-dimensional network of extracellular macromolecules and minerals that exists between cells. An ECM protein herein may be any ECM protein known the in art. Non-limiting examples of ECM proteins include: collagen, elastin, fibronectin, and laminin. More than one ECM protein may also have increased levels in a fibrotic disorder treated herein. In some embodiments, a fibrotic disorder is characterized by increased levels of 1-10, 2-9, 3-8, 4-7, or 5-6 ECM proteins. In some embodiments, a fibrotic disorder is characterized by increased levels of 1, 2, 3, 4, 5, 6, 7, 8, 9, or 10 or more ECM proteins.

In some embodiments, a fibrotic disorder that is treated with a method provided herein is characterized by increased levels of a collagen protein. Collagens are the most abundant protein in the ECM and the human body. Collagen is produced in cells and exocytosed in precursor form (procollagen) which is then cleaved and assembled into mature collagen extracellular. Collagen proteins may be divided into several families based on the types of structures that they form, including, but not limited to: fibrillar (Types I, II, III, V, and XI collagens), facit (Types IX, XII, and XIV collagens), short chain (Types VIII and X collagens), basement membrane (Type IV), and other structures (Types VI, VII, and XIII).

ECM proteins (e.g., collagen) are produced by enzymes using amino acids. Pyruvate carboxylase activity and glutaminase activity are required for extracellular matrix protein (e.g., collagen) production in a nutrient-deficient environment. Pyruvate carboxylase converts pyruvate to oxaloacetate, and oxaloacetate is required for the synthesis of extracellular matrix proteins. Glutaminase is an aminohydrolase enzyme that produces glutamine from the TCA cycle intermediate glutamate, and glutamate is required for extracellular matrix protein (e.g., collagen) synthesis.

A low-nutrient environment has decreased concentration of one or more nutrients compared to normal conditions. The one or more nutrients that may be low include, but are not limited to: amino acids (e.g., glutamine, proline, aspartate, glutamate), TCA cycle intermediates (e.g., alpha-ketoglutarate, citrate, lactate), or metabolites (e.g., glucose, vitamins). In some embodiments, a low-nutrient environment has a decreased concentration of 1%-100%, 5%-95%, 10%-90%, 15%-85%, 20%-80%, 25%-75%, 30%-70%, 35%-65%, 40%-60%, or 45%-55% compared to normal conditions. In some embodiments, a low-nutrient environment has a decreased concentration of 1%, 5%, 10%, 15%, 20%, 25%, 30%, 35%, 40%, 45%, 50%, 55%, 60%, 65%, 70%, 75%, 80%, 85%, 90%, 95%, or 100% or more compared to normal conditions.

In some embodiments, a low-nutrient environment contains low-glutamine, low-glucose, high-lactate, or some combination thereof. A combination of low-glutamine, low-glucose, high-lactate, or some combination thereof may be any combination provided herein. In some embodiments, cell hyperproliferation and/or ECM protein hyperproduction is in a low-glutamine environment.

In some embodiments, a low-glutamine environment contains a decreased level of glutamine compared to a control. Low-glutamine may be 0.1 mM-19.9 mM glutamine, 0.5 mM-19 mM glutamine, 1.0 mM-18 mM glutamine, 2 mM-17 mM glutamine, 3 mM-16 mM glutamine, 4 mM-15 mM glutamine, 5 mM-14 mM glutamine, 6 mM-13 mM glutamine, 7 mM-12 mM glutamine, 8 mM-11 mM glutamine, or 9 mM-10 mM glutamine. In some embodiments, low-glutamine may be 0.1 mM glutamine, 0.5 mM glutamine, 1.0 glutamine, 2 mM glutamine, 3 mM glutamine, 4 mM glutamine, 5 mM glutamine, 6 mM glutamine, 7 mM glutamine, 8 mM glutamine, 9 mM glutamine, 10 mM glutamine, 11 mM glutamine, 12 mM glutamine, 13 mM glutamine, 14 mM glutamine, 15 mM glutamine, 16 mM glutamine, 17 mM glutamine, 18 mM glutamine, 19 mM glutamine, 19.5 mM glutamine, or 19.9 mM glutamine.

In some embodiments, a low-glucose environment contains a decreased level of glutamine compared to a control. Low-glucose may be 0.1 mM-9.9 mM glucose, 0.5 mM-9 mM glucose, 1.0 mM-8 mM glucose, 2 mM-7 mM glucose, 3 mM-6 mM glucose, or 4 mM-5 mM glucose. In some embodiments, low-glucose may be 0.1 mM glucose, 0.5 mM glucose, 1.0 mM glucose, 2 mM glucose, 3 mM glucose, 4 mM glucose, 5 mM glucose, 6 mM glucose, 7 mM glucose, 8 mM glucose, 9 mM glucose, 9.5 mM glucose, or 9.9 mM glucose.

In some embodiments, a high-lactate environment contains an increased level of lactate compared to a control. High-lactate may be 0.1 mM-15 mM lactate, 0.5 mM-14 mM lactate, 1.0 mM-13 mM lactate, 2 mM-12 mM lactate, 3 mM-11 mM lactate, 4 mM
10 mM lactate, 5 mM-9 mM lactate, or 6 mM-8 mM lactate. In some embodiments, low-lactate may be 0.1 mM lactate, 0.5 mM lactate, 1.0 mM lactate, 2 mM lactate, 3 mM lactate, 4 mM lactate, 5 mM lactate, 6 mM lactate, 7 mM lactate, 8 mM lactate, 9 mM lactate, 10 mM lactate, 11 mM lactate, 12 mM lactate, 13 mM lactate, 14 mM lactate, or 15 mM lactate.

Treating a fibrotic disorder with methods or compositions of the present disclosure results in amelioration of ECM protein hyperproduction. Amelioration of ECM protein hyperproduction is production of an ECM protein that is decreased compared to a fibrotic cell that has not been treated with methods or composition of the present disclosure.

Amelioration of ECM protein hyperproduction may be ECM protein production that is decreased 5%-100%, 10%-95%, 20%-90%, 30%-80%, 40%-70%, or 50%-60%. In some embodiments, amelioration of ECM protein hyperproduction is production of an ECM is production that is decreased 5%, 10%, 15%, 20%, 25%, 30%, 35%, 40%, 45%, 50%, 55%, 60%, 65%, 70%, 75%, 80%, 85%, 90%, 95%, 100% or more.

### Cancer

In some aspects, the present disclosure provides a method of treating a cancer by administering to a subject in need thereof an antagonist of PC, an antagonist of glutaminase, or an antagonist of PC and an antagonist of glutaminase in an amount effective to treat the cancer. More than one million patients in the United States are diagnosed with cancer each year, and cancer remains the second-highest cause of death in the United States. About 13% of all deaths each year globally are due to cancer.

Cancer is a disease characterized by uncontrolled proliferation and spread of cancer cells, or decreased apoptosis of cancer cells. Cancer cells may coalesce in a tumor. A tumor is a solid, abnormal growth of cells (e.g., cancer cells) that may be benign or malignant. A malignant (e.g., cancerous) tumor is containing cancer cells, immune cells (e.g., T cell, dendritic cell), fibroblasts, extracellular molecules, and blood vessels.

Treating a cancer may be inhibiting tumor cells. Inhibiting tumor cells may be inhibiting tumor cell (e.g., fibroblast) proliferation, increasing tumor cell death, inhibiting the growth of tumor cells, inhibiting the metastasis (e.g., movement) of tumor cells, or any other measure of treating cancer known in the art.

In some embodiments, a tumor cell that is treated with a method provided herein is characterized by hyperproliferation of a tumor cell (e.g., fibroblast). Hyperproliferation is increased cell growth, cell division, cell movement, or a combination thereof compared to a cell that is not cancerous or subject that does not have cancer. Hyperproliferation may be increased cell growth, cell division, cell movement, or a combination thereof that is increased 5%-100%, 10%-95%, 20%-90%, 30%-80%, 40%-70%, or 50%-60%. In some embodiments, hyperproliferation is increased cell growth, cell division, cell movement, or a combination thereof that is increased 5%, 10%, 15%, 20%, 25%, 30%, 35%, 40%, 45%, 50%, 55%, 60%, 65%, 70%, 75%, 80%, 85%, 90%, 95%, 100% or more.

In some embodiments, inhibition of tumor cell proliferation results from reduction of ECM protein Reduction of ECM protein production may be reduction of ECM transcription, translation, deposition, or a combination thereof. Reduction of ECM protein production may be ECM protein production that is decreased 5%-100%, 10%-95%, 20%-90%, 30%-80%, 40%-70%, or 50%-60%. In some embodiments, reduction of ECM protein production is ECM protein production that is decreased 5%, 10%, 15%, 20%, 25%, 30%, 35%, 40%, 45%, 50%, 55%, 60%, 65%, 70%, 75%, 80%, 85%, 90%, 95%, 100% or more.

In some embodiments, a cancer is characterized by a low-nutrient environment.

Characterized by a low-nutrient environment means that a cancer (e.g., a cell, a tumor) has lower levels of a nutrient compared to a control. A control may be a cancer cell that is not treated with a method provided herein or a cell that is not cancerous (e.g., a normal cell). In some embodiments, a low-nutrient environment is low-glutamine, low-glucose, high-lactate, or some combination thereof. A low-glutamine environment may be any low-glutamine level provided herein. A low-glucose environment may be any low-glucose level provided herein.

A high-lactate environment may be any high-lactate level provided herein.

In some embodiments, methods and compositions of the present disclosure inhibit carcinogenesis. Carcinogenesis is the formation of a cancer cell, proliferation of a cancer cell (e.g., cancer cell growth, cancer cell division, cancer cell metastasis). Carcinogenesis may be due to accumulation of mutations in proteins (e.g., tumor suppressor proteins, DNA repair proteins, proto-oncogenes), extra-cellular growth signals (e.g., cytokines), or a combination thereof. In some embodiments, carcinogenesis (e.g., cancer) is in a low-nutrient environment. In some embodiments, carcinogenesis is in a low-glutamine environment, a low-glucose environment, a high-lactate environment, or some combination thereof.

A cancer may be any cancer known in the art. Non-limiting examples of cancer that may be treated with methods and compositions of the present disclosure include: carcinoma, a sarcoma, a breast cancer, a pancreatic cancer, a lung cancer, a leukemia, a lymphoma, a brain cancer, a melanoma, a liver cancer, a stomach cancer, a small intestine cancer, a large intestine cancer, a kidney cancer, a uterine cancer, an ovarian cancer, a bladder cancer, or a bone cancer.

A cancer treated with a method provided herein may be a primary cancer or a secondary cancer. A primary cancer is a cancer that is confined to the original location where the cancer began (e.g., breast, colon, etc.), and a secondary cancer is a cancer that originated in a different location and metastasized. A cancer treated with a method provided herein may be a first occurrence of the cancer or may be a subsequent occurrence of the cancer (relapsed or recurrent cancer).

Cells require energy and macromolecules to grow, divide, and move. Due to increased rates of growth, division, and movement, cancer cells require more energy and macromolecules than normal (e.g., non-cancerous) cells. Pyruvate carboxylase activity and glutaminase activity are required for energy production and macromolecule synthesis under nutrient-deficient conditions. Pyruvate carboxylase converts pyruvate to oxaloacetate, and oxaloacetate is required for energy production and synthesis of macromolecules (e.g., proteins, nucleic acids). Glutaminase is an aminohydrolase enzyme that produces glutamine from the TCA cycle intermediate glutamate, and glutamate is required for protein production.

Treating a cancer (e.g., inhibiting a tumor cell) with methods or compositions of the present disclosure results in an amelioration of cell hyperproliferation. An amelioration of cell hyperproliferation is decreased cell growth, cell division, decreased cell growth, or a combination thereof of a tumor cell that is decreased compared to a tumor cell that has not been treated with methods or composition of the present disclosure. An amelioration of cell hyperproliferation may be tumor cell proliferation that is decreased 5%-100%, 10%-95%, 20%-90%, 30%-80%, 40%-70%, or 50%-60%. In some embodiments, an amelioration of cell hyperproliferation is proliferation that is decreased 5%, 10%, 15%, 20%, 25%, 30%, 35%, 40%, 45%, 50%, 55%, 60%, 65%, 70%, 75%, 80%, 85%, 90%, 95%, 100% or more.

### Subjects

Methods provided herein may be used to treat a subject in need thereof. A subject in need thereof may have any disease or disorder provided herein including, but not limited to, a fibrotic disease (e.g., pulmonary fibrosis, liver fibrosis, kidney fibrosis) and a cancer (e.g., adenocarcinoma, carcinoma, leukemia, glioma). A subject may have one or more diseases or disorders provided herein. In some embodiments, a subject has 1-10 diseases or disorders, 2-9 diseases or disorders, 3-8 diseases or disorders, 4-7 diseases or disorders, or 5-6 diseases or disorders. In some embodiments, a subject has 1, 2, 3, 4, 5, 6, 7, 8, 9, or 10 or more diseases or disorders provided herein.

In some embodiments where a subject has a fibrotic disorder, the subject has a cell that is hyperproliferative, has hyperproduction of an extracellular matrix protein (e.g., collagen), or a cell that is hyperproliferative and has hyperproduction of an extracellular matrix protein. Hyperproliferative means that a cell has increased growth, increased division, increased movement, or some combination thereof. Increased growth, increased division, and/or increased growth is relative to a control cell. Hyperproduction of an extracellular matrix protein (e.g., collagen) means that a cell produces increased levels of an extracellular matrix protein compared to a control cell. A control cell may be a cell from the same subject that is not hyperproliferative, does not have hyperproduction of collagen, and/or is not hyperproliferative and does not have hyperproduction of collagen or a cell from a different subject that does not have a fibrotic disorder.

Hyperproliferation may be proliferation (e.g., growth, division, movement) that is increased 5%-100%, 10%-90%, 20%-80%, 30%-70%, 40%-60%, or 50%-60% compared to a control. In some embodiments, proliferation is increased 5%, 10%, 15%, 20%, 25%, 30%, 35%, 40%, 45%, 50%, 55%, 60%, 65%, 70%, 75%, 80%, 85%, 90%, 95%, 100% or more, compared to a control.

In some embodiments where a subject has a cancer, the subject has a cell that is hyperproliferative, has hyperproduction of an extracellular matrix protein (e.g., collagen), or a cell that is hyperproliferative and has hyperproduction of an extracellular matrix protein. A control cell may be a cell from the same subject that is not hyperproliferative, does not have hyperproduction of collagen, and/or is not hyperproliferative and does not have hyperproduction of collagen or a cell from a different subject that does not have a cancer.

In some embodiments, a subject is administered an effective amount of an antagonist of pyruvate carboxylase (PC), an antagonist of glutaminase, or an antagonist of PC and an antagonist of glutaminase to treat a disease or disorder. An effective amount of an antagonist of PC, antagonist of glutaminase, or an antagonist of PC and an antagonist of glutaminase is any amount that decreases cell proliferation, decreases cell survival, decreases protein synthesis, decreases extracellular matrix (ECM) protein deposition, decreases fibrosis, or a combination thereof.

An effective amount of an antagonist of PC or an antagonist of glutaminase will vary based on factors that are known to a person skilled in the art, including, but not limited to: age of a subject, height of a subject, weight of a subject, pre-existing conditions, stage of a disease or disorder, other treatments or medications that a subject is being administered, or a combination thereof. In some embodiments, an effective amount of an antagonist of PC is 1 μg/kg-1,000 mg/kg, 10 μg/kg-100 mg/kg, 100 μg/kg-10 mg/kg, or 500 μg/kg-1 mg/kg. In some embodiments, an effective amount of an antagonist of PC is 1 μg/kg, 10 μg/kg, 25 μg/kg, 50 μg/kg, 75 μg/kg, 100 μg/kg, 200 μg/kg, 250 μg/kg, 300 μg/kg, 350 ug pg/kg. 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1 mg/kg, 10 mg/kg, 25 mg/kg, 50 mg/kg, 75 mg/kg, 100 mg/kg, 200 mg/kg, 250 mg/kg, 300 mg/kg, 350 ug mg/kg. 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1,000 mg/kg. In some embodiments, an effective amount of an antagonist of glutaminase is 1 μg/kg-1,000 mg/kg, 10 μg/kg-100 mg/kg, 100 μg/kg-10 mg/kg, or 500 μg/kg-1 mg/kg. In some embodiments, an effective amount of an antagonist of glutaminase is 1 μg/kg, 10 μg/kg, 25 μg/kg, 50 μg/kg, 75 μg/kg, 100 μg/kg, 200 μg/kg, 250 μg/kg, 300 μg/kg, 350 ug pg/kg. 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1 mg/kg, 10 mg/kg, 25 mg/kg, 50 mg/kg, 75 mg/kg, 100 mg/kg, 200 mg/kg, 250 mg/kg, 300 mg/kg, 350 ug mg/kg. 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1,000 mg/kg.

In some embodiments, a subject is a vertebrate. A vertebrate may be any vertebrate known in the art including, but not limited to: a human, a rodent (e.g., mouse, rat, hamster), a non-human primate (e.g., Rhesus monkey, chimpanzee, orangutan), a pet (e.g., dog, cat, ferret), a livestock animal (e.g., pig, cow, sheep, chicken), or a fish (zebrafish, catfish, perch).

An antagonist of PC, an antagonist of glutaminase, or an antagonist of PC and an antagonist of glutaminase may be administered to a subject by any method known in the art. Non-limiting examples of methods for administering an antagonist of PC include: injection (e.g., intravenous, intramuscular, intraarterial), inhalation (e.g., by nebulizer, by inhaler), ingestion (e.g., oral, rectal, vaginal), sublingual or buccal dissolution, ocular placement, otic placement, and absorbed through skin (e.g., cutaneously, transdermally).

Administration of an antagonist of PC, an antagonist of glutaminase, or some combination thereof may be in vivo (e.g., into a subject) or in vitro (e.g., into a cell in cell culture medium). In some embodiments, an antagonist of PC, an antagonist of glutaminase, or some combination thereof is administered in vivo. In some embodiments, an antagonist of PC, an antagonist of glutaminase, or some combination thereof is administered in vitro.

In some embodiments, an antagonist of PC is administered simultaneously with an antagonist of glutaminase. Simultaneously means that an antagonist of PC and an antagonist of glutaminase are administered at the same time. Simultaneous administration may mean that an antagonist of PC and an antagonist of glutaminase are in the same formula (e.g., in a pharmaceutical composition) or that an antagonist of PC and an antagonist of glutaminase are in separate formulas.

In some embodiments, an antagonist of PC is administered sequentially with an antagonist of glutaminase. In these embodiments, an antagonist of PC is administered to a subject that is already receiving an antagonist of glutaminase or vice versa. A subject receiving an antagonist (e.g., of PC, or glutaminase) may be administered the antagonist by any method provided herein. In some embodiments, an antagonist of PC is administered before an antagonist of glutaminase. In some embodiments, an antagonist of glutaminase is administered before an antagonist of PC. Sequential administration may be administration that is separated by seconds, minutes, hours, days, weeks, months, or years.

### Methods for Use

Methods provided herein may be used in vitro (e.g., in a cultured cell) or in vivo (e.g., in a subject) to antagonize pyruvate carboxylase (PC), glutaminase, or PC and glutaminase. As described above, PC and glutaminase are required for cell proliferation and extracellular matrix (ECM) protein (e.g., collagen) production in a nutrient-replete environment. Because PC and glutaminase are required for cell proliferation and ECM protein production, methods provided herein may be used to inhibit cell proliferation and ECM protein production.

### Inhibiting Cell Proliferation

Methods and compositions provided herein may be used to inhibit cell proliferation (e.g., a tumor cell, a fibroblast). Inhibiting cell proliferation may be decreased cell growth, decreased cell division, decreased cell movement, or some combination thereof.

In some embodiments, an antagonist of PC, an antagonist of glutaminase, or an antagonist of PC and an antagonist of glutaminase is administered to a subject or contacted with a cell to inhibit cell proliferation. Contacting a cell with an antagonist (e.g., of PC, of glutaminase, or a combination thereof) may be through any method known in the art. Non-limiting methods of contacting a cell with an antagonist include: introducing an antagonist (e.g., of PC, of glutaminase, or a combination thereof) into a cell culture medium or injecting an antagonist into a cell.

An antagonist of PC inhibits cell proliferation because PC produces the TCA cycle intermediate oxaloacetate from pyruvate. The TCA cycle is the main source of energy for cells. The products of the TCA cycle, including, but not limited to, NADH, GTP, and ATP, are used in other cell processes such as cell growth, cell division, and macromolecule (e.g., proteins, nucleic acids) synthesis. Thus, an antagonist of PC inhibits cell proliferation by inhibiting production of cellular energy and macromolecule synthesis that is required for cell growth and cell division. An antagonist of glutaminase inhibits cell proliferation because glutaminase produces glutamine from glutamate. Glutamine is an amino acid that is used in protein (e.g., extracellular matrix protein) synthesis. Thus, an antagonist of glutaminase inhibits cell proliferation by inhibiting production of proteins.

A subject may be any subject described herein. In some embodiments, a subject is a subject having a fibrotic disorder (e.g., pulmonary fibrosis, liver fibrosis). In some embodiments, a subject is a subject having a cancer (e.g., carcinoma, sarcoma, breast cancer).

A subject may also have a fibrotic disorder and a cancer. In some embodiments, a cell is a cell derived from a subject having a fibrotic disorder or from a subject having a cancer. Derived from may be directly obtained from a subject or indirectly obtained from a subject (e.g., after a period of in vitro cell culture). In some embodiments, a cell is derived from a subject having a fibrotic disorder and a cancer.

Cell proliferation (e.g., growth, division, movement) may be measured by any method known in the art. Non-limiting methods of measuring proliferation include: metabolic activity assays (e.g., MTT, XTT, MTS, WST1), cell proliferation marker assays (e.g., Ki-67, PCNA, topoisomerase IIB, phosphorylated histone H3), ATP concentration assays (e.g., luciferase), DNA synthesis assays (e.g., BrdU, 3H-thymine), and cell movement assays (e.g., scratch assay, agarose drop assay, cell culture insert).

Proliferation may be inhibited by 5%-100%, 10%-90%, 20%-80%, 30%-70%, or 50%-60% compared to a control. In some embodiments, proliferation is inhibited by 5%, 10%, 15%, 20%, 25%, 30%, 35%, 40%, 45%, 50%, 55%, 60%, 65%, 70%, 75%, 80%, 85%, 90%, 95%, 100%, or more, compared to a control. A control is a subject or a cell that has a disorder (e.g., fibrotic disorder, cancer) and has not been administered or contacted with an antagonist of PC, an antagonist of glutaminase, or an antagonist of PC and an antagonist of glutaminase.

### Inhibiting Extracellular Matrix Protein Production

Methods and compositions provided herein may be used to inhibit extracellular matrix (ECM) protein production (e.g., collagen). Inhibiting ECM protein production may be decreased ECM protein production or decreased ECM protein deposition.

In some embodiments, an antagonist of PC is administered, an antagonist of glutaminase, or an antagonist of PC and an antagonist of glutaminase to a subject or contacted with a cell to inhibit ECM protein production. An antagonist of PC inhibits ECM protein production because PC produces the TCA cycle intermediate oxaloacetate from pyruvate. The TCA is the main source of energy for cells. The products of the TCA cycle, including, but not limited to, NADH, GTP and ATP, are used in other cell processes such as cell growth, cell division, and macromolecule (e.g., proteins, nucleic acids) synthesis.

Additionally, an antagonist of PC inhibits ECM protein (e.g., collagen) production by decreasing H3K27 acetylation of an ECM protein enhancer compared to a control. Decreasing H3K27 acetylation decreases transcription, and thus translation and production of an ECM protein. Thus, an antagonist of PC inhibits ECM protein production by inhibiting production of cellular energy and macromolecule (e.g., protein) synthesis.

An antagonist of glutaminase inhibits ECM protein production because glutaminase produces glutamine from glutamate. Glutamine is an amino acid that is used in protein (e.g., ECM protein) synthesis. Thus, an antagonist of glutaminase inhibits ECM protein production by inhibiting production of proteins.

Fibroblasts are cells that produce ECM proteins (e.g., collagen, fibronectin, laminin). In some embodiments, inhibiting ECM protein production is in a fibroblast. In some embodiments, a fibroblast is associated with a fibrotic disorder. Associated with a fibrotic disorder may mean that the fibroblast is derived from a subject having a fibrotic disorder or has characteristics consistent with a fibrotic disorder. Characteristics consistent with a fibrotic disorder include, but are not limited to, hyperproduction of ECM proteins, cell hyperproliferation, or a combination thereof.

In some embodiments, a fibroblast is in a low-glutamine environment. A low-glutamine environment contains decreased a decreased glutamine concentration compared to an environment that is not low in glutamine. A low-glutamine environment may contain 0.1%-19.9%, 1%-19%, 2%-18%, 3%-17%, 4%-16%, 5%-15%, 6%-14%, 7%-13%, 8%-12%, or 9%-11% glutamine. In some embodiments, a low-glutamine environment contains 0.1%, 0.5%, 1%, 2%, 3%, 4%, 5%, 6%, 7%, 8%, 9%, 10%, 11%, 12%, 13%, 14%, 15%, 16%, 17%, 18%, 19%, 19.5%, or 19.9% glutamine.

A subject may be any subject described herein. In some embodiments, a subject is a subject having a fibrotic disorder (e.g., pulmonary fibrosis, liver fibrosis). In some embodiments, a subject is a subject having a cancer (e.g., carcinoma, sarcoma, breast cancer). In some embodiments, a cell is a cell derived from a subject having a fibrotic disorder or from a subject having a cancer. Derived from may be directly obtained from a subject or indirectly obtained from a subject (e.g., after a period of in vitro cell culture).

Production of an extracellular matrix protein (e.g., collagen) may be measured by any method known in the art. Non-limiting methods of measuring production of ECM protein include: protein staining, isobaric demethylated leucine (DiLeu) labeling and quantification, mass spectrometry, reversed phase liquid chromatography, second harmonic generation (SHG) microscopy, and strong cation exchange chromatography. In some embodiments, ECM proteins are measured by protein staining. Non-limiting examples of protein staining of ECM proteins include: Picrosirius Red staining, Masson's Trichrome staining, and hematoxylin and eosin staining.

ECM protein production may be inhibited by 5%-100%, 10%-90%, 20%-80%, 30%-70%, or 50%-60% compared to a control. In some embodiments, ECM protein production is inhibited by 5%, 10%, 15%, 20%, 25%, 30%, 35%, 40%, 45%, 50%, 55%, 60%, 65%, 70%, 75%, 80%, 85%, 90%, 95%, 100%, or more, compared to a control. A control is a subject or a cell that has not been administered or contacted with an antagonist of PC, an antagonist of glutaminase, or an antagonist of PC and an antagonist of glutaminase.

### Compositions

The present disclosure demonstrates that PC and glutaminase are required for cell proliferation and ECM protein production in a nutrient-replete environment, including a nutrient-replete cell culture medium. Cells contacted with an antagonist of PC in a nutrient-deficient environment, an antagonist of glutaminase in a nutrient-deficient environment, or an antagonist of PC and an antagonist of glutaminase in nutrient-replete cell culture medium will have reduced proliferation and ECM protein production. Thus, the present disclosure provides, in some embodiments, a pharmaceutical composition comprising an antagonist of PC, an antagonist of glutaminase, or a combination thereof. The antagonist of PC and antagonist of glutaminase may be any antagonist of PC and antagonist of glutaminase provided herein. In some embodiments, the composition further comprises a nutrient-deficient cell culture medium and/or a nutrient-replete cell culture medium.

Nutrient-deficient cell culture medium is cell culture medium deficient in one or more nutrients required for cellular processes, including but not limited to: amino acids, vitamins, and ions. Deficient in one or more amino acids means that the cell culture medium does not contain sufficient levels of one or more amino acids to support cellular processes. The cellular processes that are not supported in nutrient-deficient cell culture medium may be cell proliferation, survival, ECM protein production, ECM protein deposition, or a combination thereof.

In some embodiments, nutrient-deficient cell culture medium has a decreased concentration of 1%-100%, 5%-95%, 10%-90%, 15%-85%, 20%-80%, 25%-75%, 30%-70%, 35%-65%, 40%-60%, or 45%-55% compared to normal conditions. In some embodiments, nutrient-deficient cell culture medium has a decreased concentration of 1%, 5%, 10%, 15%, 20%, 25%, 30%, 35%, 40%, 45%, 50%, 55%, 60%, 65%, 70%, 75%, 80%, 85%, 90%, 95%, or 100% or more compared to normal conditions.

Nutrient-deficient cell culture medium may be deficient in any amino acid including, but not limited to, arginine, alanine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or any combination thereof. In some embodiments, nutrient-deficient cell culture medium is deficient in 1-20, 2-19, 3-18, 4-17, 5-16, 6-15, 7-14, 8-13, 9-12, or 10-11 amino acids. In some embodiments, nutrient-deficient cell culture medium is deficient in 1, 2, 3, 4, 5, 6, 7, 8, 9, 10, 11, 12, 13, 14, 15, 16, 17, 18, 19, or 20 amino acids. In some embodiments, nutrient-deficient cell culture medium is deficient in glutamine, glucose, lactate, or some combination thereof at any concentration provided herein.

In some embodiments, a nutrient-deficient environment contains low-glutamine.

Low-glutamine may be 0.1 mM-19.9 mM glutamine, 0.5 mM-19 mM glutamine, 1.0 mM-18 mM glutamine, 2 mM-17 mM glutamine, 3 mM-16 mM glutamine, 4 mM-15 mM glutamine, 5 mM-14 mM glutamine, 6 mM-13 mM glutamine, 7 mM-12 mM glutamine, 8 mM-11 mM glutamine, or 9 mM-10 mM glutamine. In some embodiments, low-glutamine may be 0.1 mM glutamine, 0.5 mM glutamine, 1.0 glutamine, 2 mM glutamine, 3 mM glutamine, 4 mM glutamine, 5 mM glutamine, 6 mM glutamine, 7 mM glutamine, 8 mM glutamine, 9 mM glutamine, 10 mM glutamine, 11 mM glutamine, 12 mM glutamine, 13 mM glutamine, 14 mM glutamine, 15 mM glutamine, 16 mM glutamine, 17 mM glutamine, 18 mM glutamine, 19 mM glutamine, 19.5 mM glutamine, or 19.9 mM glutamine.

In some embodiments, a nutrient-deficient cell culture medium contains low-glucose. Low-glucose may be 0.1 mM-9.9 mM glucose, 0.5 mM-9 mM glucose, 1.0 mM-8 mM glucose, 2 mM-7 mM glucose, 3 mM-6 mM glucose, or 4 mM-5 mM glucose. In some embodiments, low-glucose may be 0.1 mM glucose, 0.5 mM glucose, 1.0 mM glucose, 2 mM glucose, 3 mM glucose, 4 mM glucose, 5 mM glucose, 6 mM glucose, 7 mM glucose, 8 mM glucose, 9 mM glucose, 9.5 mM glucose, or 9.9 mM glucose.

In some embodiments, a nutrient-deficient cell culture medium contains high-lactate. high-lactate may be 0.1 mM-15 mM lactate, 0.5 mM-14 mM lactate, 1.0 mM-13 mM lactate, 2 mM-12 mM lactate, 3 mM-11 mM lactate, 4 mM-10 mM lactate, 5 mM-9 mM lactate, or 6 mM-8 mM lactate. In some embodiments, a nutrient-deficient environment contains 0.1 mM lactate, 0.5 mM lactate, 1.0 mM lactate, 2 mM lactate, 3 mM lactate, 4 mM lactate, 5 mM lactate, 6 mM lactate, 7 mM lactate, 8 mM lactate, 9 mM lactate, 10 mM lactate, 11 mM lactate, 12 mM lactate, 13 mM lactate, 14 mM lactate, or 15 mM lactate.

Nutrient-replete cell culture medium is cell culture medium replete in one or more nutrients required for cellular processes, including but not limited to: amino acids, vitamins, and ions. Replete in one or more amino acids means that the cell culture medium contains sufficient levels of one or more amino acids to support cellular processes. The cellular processes that are supported in nutrient-replete cell culture medium may be cell proliferation, survival, ECM protein production, ECM protein deposition, or a combination thereof.

In some embodiments, nutrient-replete cell culture medium has an increased concentration of 1%-100%, 5%-95%, 10%-90%, 15%-85%, 20%-80%, 25%-75%, 30%-70%, 35%-65%, 40%-60%, or 45%-55% compared to normal conditions. In some embodiments, nutrient-replete cell culture medium has an increased concentration of 1%, 5%, 10%, 15%, 20%, 25%, 30%, 35%, 40%, 45%, 50%, 55%, 60%, 65%, 70%, 75%, 80%, 85%, 90%, 95%, or 100% or more compared to normal conditions.

Nutrient-replete cell culture medium may be replete in any amino acid including, but not limited to, arginine, alanine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or any combination thereof. In some embodiments, nutrient-replete cell culture medium is replete in 1-20, 2-19, 3-18, 4-17, 5-16, 6-15, 7-14, 8-13, 9-12, or 10-11 amino acids. In some embodiments, nutrient-replete cell culture medium is replete in 1, 2, 3, 4, 5, 6, 7, 8, 9, 10, 11, 12, 13, 14, 15, 16, 17, 18, 19, or 20 amino acids. In some embodiments, nutrient-replete cell culture medium is replete in glutamine, glucose, lactate, or some combination thereof at any concentration provided herein.

In some embodiments, a nutrient-replete environment contains glutamine. Glutamine may be 20 mM-50 mM glutamine, 25 mM-45 mM glutamine, or 30 mM-40 mM glutamine. In some embodiments, glutamine may be 20 mM glutamine, 21 mM glutamine, 22 glutamine, 23 mM glutamine, 24 mM glutamine, 25 mM glutamine, 26 mM glutamine, 27 mM glutamine, 28 mM glutamine, 29 mM glutamine, 30 mM glutamine, 31 mM glutamine, 32 mM glutamine, 33 mM glutamine, 34 mM glutamine, 35 mM glutamine, 36 mM glutamine, 37 mM glutamine, 38 mM glutamine, 39 mM glutamine, 40 mM glutamine, 41 mM glutamine, 42 mM glutamine, 43 mM glutamine, 44 mM glutamine, 45 mM glutamine, 46 mM glutamine, 47 mM glutamine, 48 mM glutamine, 49 mM glutamine, or 50 mM or more glutamine.

In some embodiments, a nutrient-replete cell culture medium contains glucose. Glucose may be 10 mM-50 mM glucose, 15 mM-45 mM glucose, 20 mM-40 mM glucose, or 25 mM-35 mM glucose. In some embodiments, glucose may be 10 mM glucose, 11 mM glucose, 12 mM glucose, 13 mM glucose, 14 mM glucose, 15 mM glucose, 16 mM glucose, 17 mM glucose, 18 mM glucose, 19 mM glucose, 20 mM glucose, 21 mM glucose, 22 mM glucose, 23 mM glucose, 24 mM glucose, 25 mM glucose, 26 mM glucose, 27 mM glucose, 28 mM glucose, 29 mM glucose, 30 mM glucose, 31 mM glucose, 32 mM glucose, 33 mM glucose, 34 mM glucose, 35 mM glucose, 36 mM glucose, 37 mM glucose, 38 mM glucose, 39 mM glucose, 40 mM glucose, 41 mM glucose, 42 mM glucose, 43 mM glucose, 44 mM glucose, 45 mM glucose, 46 mM glucose, 47 mM glucose, 48 mM glucose, 49 mM glucose, or 50 mM or more glucose.

In some embodiments, a nutrient-replete cell culture medium contains lactate. Lactate may be 15 mM-50 mM lactate, 20 mM-45 mM lactate, 25 mM-40 mM lactate, or 30 mM-35 mM lactate. In some embodiments, lactate may be 15 mM lactate, 16 mM lactate, 17 mM lactate, 18 mM lactate, 19 mM lactate, 20 mM lactate, 21 mM lactate, 22 mM lactate, 23 mM lactate, 24 mM lactate, 25 mM lactate, 26 mM lactate, 27 mM lactate, 28 mM lactate, 29 mM lactate, 30 mM lactate, 31 mM lactate, 32 mM lactate, 33 mM lactate, 34 mM lactate, 35 mM lactate, 36 mM lactate, 37 mM lactate, 38 mM lactate, 39 mM lactate, 40 mM lactate, 41 mM lactate, 42 mM lactate, 43 mM lactate, 44 mM lactate, 45 mM lactate, 46 mM lactate, 47 mM lactate, 48 mM lactate, 49 mM lactate, or 50 mM or more lactate.

A nutrient-deficient cell culture medium and a nutrient-replete cell culture medium provided herein may contain one or more additives. Additives are exogenous compounds that are added to a nutrient-deficient or nutrient-replete medium. An additive may be any compound known in the art to be added to cell medium. Non-limiting examples of classes of compounds that are added to cell medium include: antibiotics (e.g., streptomycin, penicillin, ampicillin, kanamycin), serum (e.g., bovine serum albumin, human serum albumin, fetal bovine serum), amino acids (e.g., arginine, alanine, asparagine, aspartic acid, cysteine, glutamic acid, glutamine, glycine, histidine, isoleucine, leucine, lysine, methionine, phenylalanine, proline, serine, threonine, tryptophan, tyrosine, valine), inorganic salt (e.g., ammonium molybdate, ammonium metavandate, calcium chloride, cupric sulfate, ferric nitrate, ferrous sulfate, manganese sulfate, magnesium chloride, magnesium sulfate, nickel chloride, potassium chloride, sodium metasilicate, sodium selenite, sodium phosphate dibasic, sodium phosphate monobasic, stannous chloride, zinc sulfate), vitamins (e.g., biotin, choline chloride, folic acid, myo-inositol, niacinamide, pantothenic acid, pyridoxal, pyridoxine, riboflavin, thiamine, vitamin B12), and buffers (e.g., glucose, HEPES, hypoxanthine, linoleic acid, Phenol Red, putrescine, pyruvic acid, thioctic acid, thymidine, sodium bicarbonate).

In some embodiments, nutrient-deficient cell culture medium and nutrient-replete cell culture medium contains serum, penicillin, and streptomycin. The concentration of serum, penicillin, and streptomycin may be any concentration in cell culture medium known in the art. In some embodiments, nutrient-deficient cell culture medium and nutrient-replete cell culture medium contains 1%-30%, 2%-29%, 3%-28%, 4%-27%, 5%-26%, 6%-25%, 7%-24%, 8%-23%, 9%-22%, 10%-21%, 11%-20%, 12%-19%, 13%-18%, 14%-17%, or 15%-16% serum. In some embodiments, nutrient-deficient cell culture medium and nutrient-replete cell culture medium contains 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% serum. In some embodiments, nutrient-deficient cell culture medium and nutrient-replete cell culture medium contains 10 units/mL-150 units/mL, 20 units/mL-140 units/mL, 30 units/mL-130 units/mL, 40 units/mL-120 units/mL, 50 units/mL-110 units/mL, 60 units/mL-100 units/mL, or 70 units/mL-90 units/mL penicillin. In some embodiments, nutrient-deficient cell culture medium contains 10 units/mL, 20 units/mL, 30 units/mL, 40 units/mL, 50 units/mL, 60 units/mL, 70 units/mL, 80 units/mL, 90 units/mL, 100 units/mL, 110 units/mL, 120 units/mL, 130 units/mL, 140 units/mL, or 150 units/mL penicillin. In some embodiments, nutrient-deficient cell culture medium and nutrient-replete cell culture medium contains 10 μg/mL-150 μg/mL, 20 μg/mL-140 μg/mL, 30 μg/mL-130 μg/mL, 40 μg/mL-120 μg/mL, 50 μg/mL-110 μg/mL, 60 μg/mL-100 μg/mL, or 70 μg/mL-90 μg/mL streptomycin. In some embodiments, nutrient-deficient cell culture medium and nutrient-replete cell culture medium contains 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, or 150 μg/mL streptomycin.

In some embodiments, a composition further contains cells. The cells may be any cell expressing PC and glutaminase. Non-limiting examples of cells contained in a composition including, but not limited to: fibroblasts, tumor cells (e.g., derived from a carcinoma, a sarcoma, a breast cancer, a pancreatic cancer, a lung cancer, a leukemia, a lymphoma, a brain cancer, a melanoma, a liver cancer, a stomach cancer, a small intestine cancer, a large intestine cancer, a kidney cancer, a uterine cancer, an ovarian cancer, a bladder cancer, or a bone cancer), epithelial cells, blood cells, bone cells, neurons, and immune cells.

A composition may contain 1×10²-1×10²⁰ cells, 1×10³-1×10¹⁹ cells, 1×10⁴-1×10¹⁸ cells, 1×10⁵-1×10¹⁷ cells, 1×10⁶-1×10¹⁶ cells, 1×10⁷-1×10¹⁵ cells, 1×10⁸-1×10¹⁴ cells, 1×10⁹-1×10¹⁵ cells, 1×10¹⁰-1×10¹⁴ cells, or 1×10¹¹-1×10¹³ cells. In some embodiments, a composition contains 1×10² cells, 1×10³ cells, 1×10⁴ cells, 1×10⁵ cells, 1×10⁶ cells, 1×10⁷ cells, 1×10⁸ cells, 1×10⁹ cells, 1×10¹⁰ cells, 1×10¹¹ cells, 1×10¹² cells, 1×10¹³ cells, 1×10¹⁴ cells, 1×10¹⁵ cells, 1×10¹⁶ cells, 1×10¹⁷ cells, 1×10¹⁸ cells, 1×10¹⁹ cells, 1×10²⁰ cells or more.

### Kits

The present disclosure provides, in some embodiments, a kit comprising an antagonist of PC, an antagonist of glutaminase, and instructions for use of the antagonist of PC and the antagonist of glutaminase. An antagonist of PC and an antagonist of glutaminase may be any antagonist of PC and antagonist of glutaminase described herein.

A kit may have a single container containing an antagonist of PC and an antagonist of glutaminase. In some embodiments, a kit has multiple containers. In some embodiments, a kit has a first container containing an antagonist of PC and a second container containing an antagonist of glutaminase. A container may be a vessel known in the art to house a small molecule, a nucleic acid, a polypeptide, or a protein. Non-limiting examples of containers that may be used in kits of the present disclosure include: a vial, a syringe, a tube, a pouch, a bottle, a cuvette, or a syrette. In some embodiments, a kit comprises a vial, a syringe, or some combination thereof.

Instructions for use of an antagonist of PC and an antagonist of glutaminase may be instructions for contacting a cell (e.g., a fibroblast, a tumor cell) with an antagonist of PC, an antagonist of glutaminase, or a combination thereof; administering to a subject an antagonist of PC, an antagonist of glutaminase, or a combination thereof; or culturing a cell (e.g., a fibroblast, a tumor cell) in a nutrient-deficient cell culture medium and contacting the cell with an antagonist of PC, an antagonist of glutaminase, or a combination thereof.

### EXAMPLES

### Example 1: Glutamine-Dependent TCA Cycle Anaplerosis is Dependent for TGFβ Induced Collagen Synthesis

The aberrant production of collagen by fibroblasts is a hallmark of many solid tumors and can influence cancer progression. How the mesenchymal cells in the tumor microenvironment maintain their production of extracellular matrix proteins as the vascular delivery of glutamine and glucose becomes compromised remains unclear. As described herein, pyruvate carboxylase (PC)-mediated anaplerosis in tumor-associated fibroblasts was shown to contribute to tumor fibrosis and growth. Using cultured mesenchymal and cancer cells, as well as mouse allograft models, data is provided herein that extracellular lactate can be utilized by fibroblasts to maintain tricarboxylic acid (TCA) cycle anaplerosis and non-essential amino acid biosynthesis through PC activity. Furthermore, as described herein fibroblast PC was required for collagen production in the tumor microenvironment. These results established TCA cycle anaplerosis as a determinant of extracellular matrix collagen production, and identified PC as a potential target to inhibit tumor desmoplasia.

Fibroblasts are mesenchymal cells that play an integral part in the wound healing response. Following disruption of tissue homoeostasis, resting fibroblasts are recruited to the site of injury where they become activated by profibrotic stimuli to upregulate the synthesis and secretion of extracellular matrix (ECM) proteins such as collagen to promote regeneration of the damaged parenchymal. Cancer arises from oncogenic mutations in parenchymal cells, leading to cellular transformation and excessive cell proliferation². This insult to the functional parenchyma disrupts normal tissue homoeostasis and initiates a tissue repair response that involves the activation of fibroblasts by growth factors such as transforming growth-factor beta (TGFβ ), resulting in the development of a desmoplastic tumor stroma characterized by excessive deposition of ECM, including collagens¹. In some tumors such as breast cancer (BRCA) and pancreatic ductal adenocarcinoma (PDAC), the desmoplastic stroma comprises up to 90% of tumor mass³, and the aberrant presence of ECM proteins or enrichment of an ECM expression signature is associated with poor prognosis across cancer types⁴⁻⁶. Previous studies have shown that the ECM modulates virtually all hallmarks of cancer⁷; for example, increased collagen I density promotes mammary tumor initiation, growth and invasion⁸. Thus, targeting ECM synthesis in stromal fibroblasts has emerged as a potential strategy to limit cancer progression in desmoplastic tumors.

In vitro, the ability of profibrotic stimuli to promote ECM synthesis has been supported by metabolic reprogramming⁹. For example, TGFβ promotes the uptake of glucose and glutamine and their utilization for glycine and proline biosynthesis, respectively, to meet the demand for glycine and proline imposed by excessive collagen synthesis^{10, 11}. In addition, TGFβ increases the mitochondrial oxidation of glucose and glutamine carbon to support the bioenergetic demand of increased translation of ECM proteins¹¹. Thus, an abundant supply of these major nutrients as present in standard cell culture media can support a high rate of ECM synthesis. However, glucose and glutamine concentrations in human plasma are lower compared to what has been used in vitro¹², and their concentrations in the microenvironments of many tumors and certain types of healing wounds are further reduced, due to vascular compromise and/or excess tumor cell nutrient consumption¹³. For example, glutamine levels profoundly drop following tissue injury¹⁴, and glutamine and glucose are among the most depleted nutrients in tumors¹⁵⁻¹⁷.

It remains unclear how the mesenchymal cells in the tumor microenvironment maintain their production of ECM proteins as the vascular delivery of glutamine and glucose becomes compromised. Here, it is shown that extracellular lactate can be utilized to maintain TCA cycle anaplerosis and non-essential amino acid biosynthesis through the activity of PC, and that fibroblast PC is required for collagen production in the tumor microenvironment.

### Results

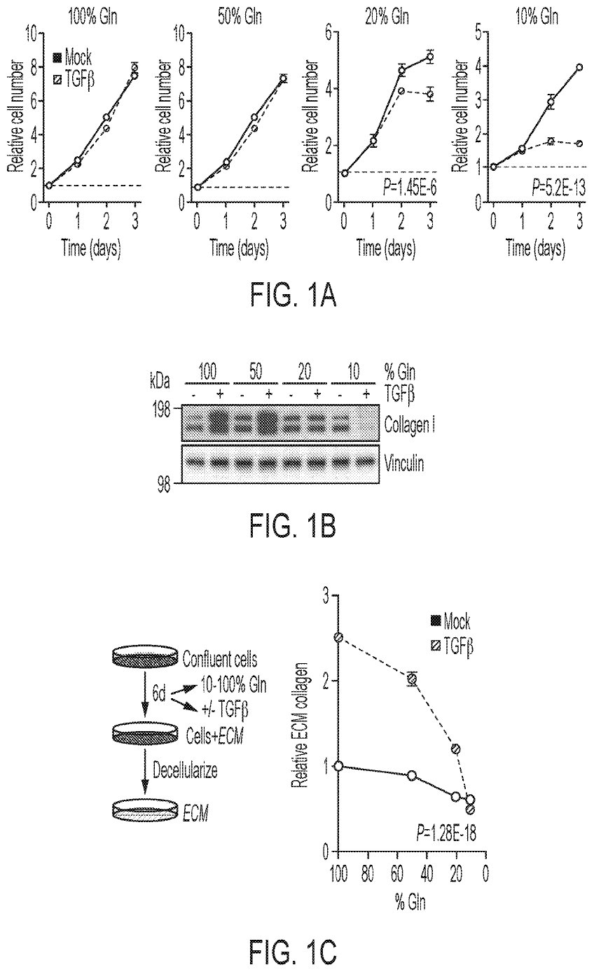

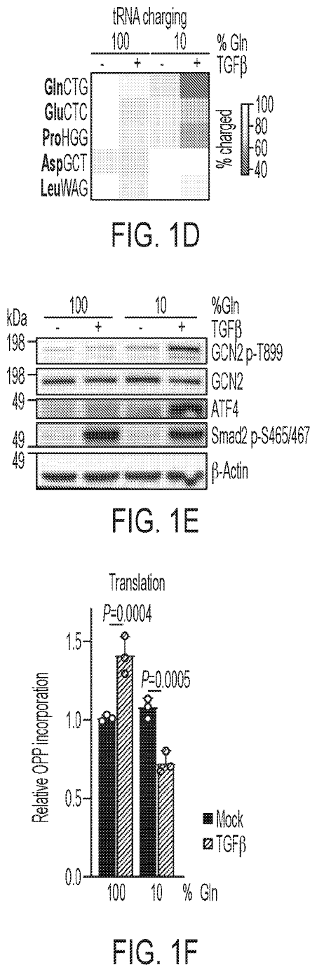

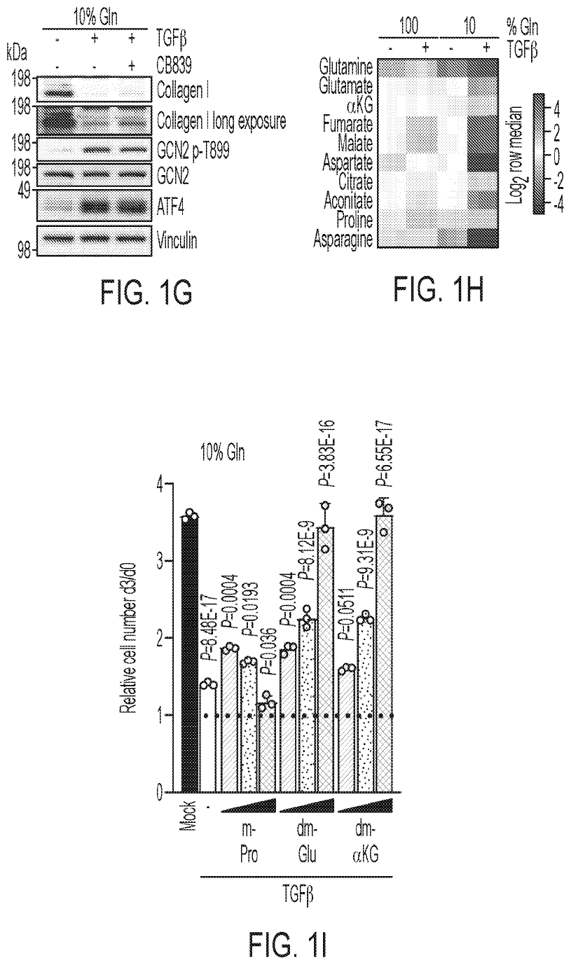

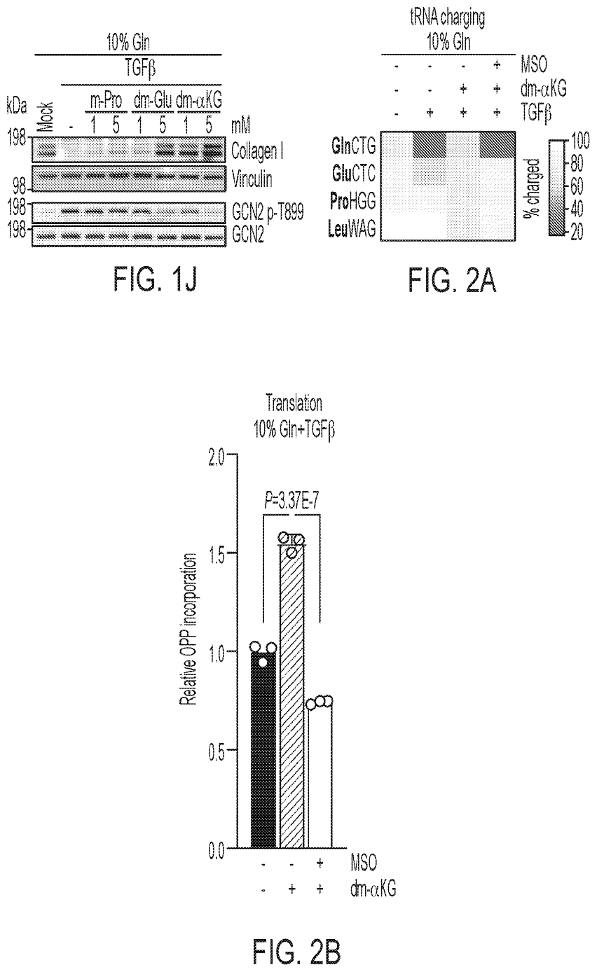

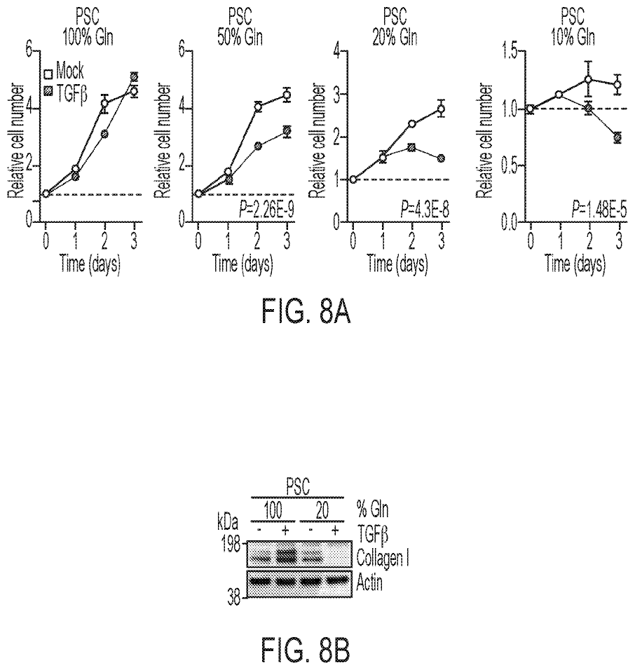

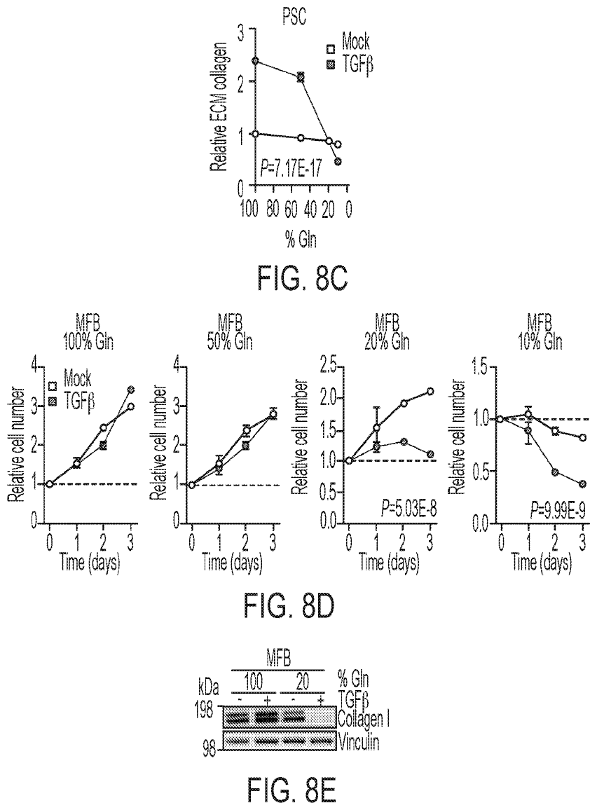

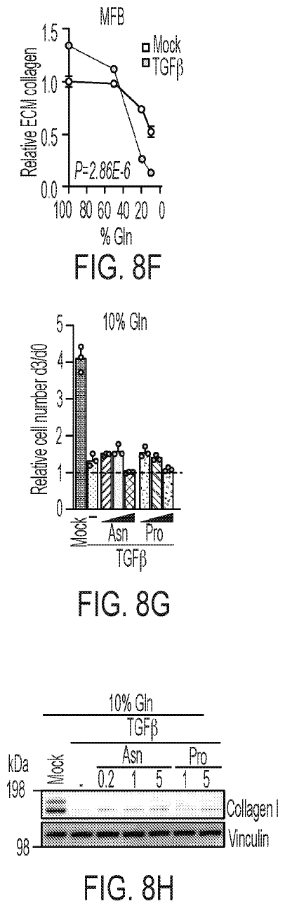

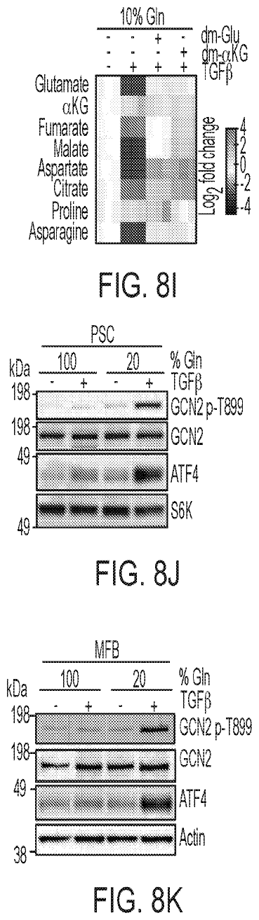

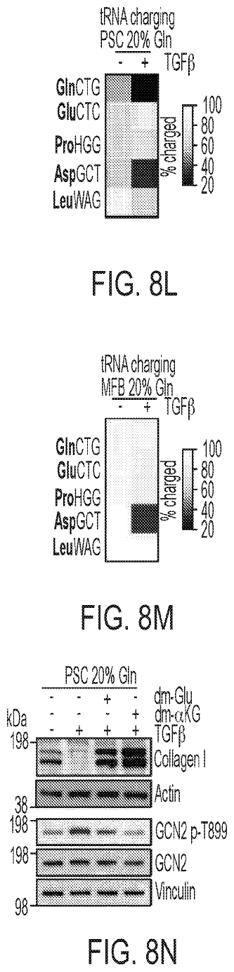

TGFβ-induced collagen synthesis is glutamine dependent. To investigate the response of fibroblasts to glutamine limitation, NIH-3T3 cells were cultured in media with glutamine (Gln) concentrations ranging from 2 mM (100%) to 0.2 mM (10%), in the presence or absence of TGFβ. In culture medium containing 100% and 50% Gln, TGFβ stimulation did not change cell proliferation over 3 days but upregulated collagen I levels in growing cells and in the ECM secreted by confluent fibroblasts (FIGS. 1A-1C). TGFβ was no longer able to increase collagen I levels in medium containing 20% Gln (FIG. 1B). In medium containing 10% Gln, TGFβ-treated but not mock-treated fibroblasts ceased to proliferate and produce collagen I (FIGS. 1A-1C). To validate these findings in fibroblasts relevant to cancer, primary pancreatic stellate cells (PSCs) and primary mammary fibroblasts (MFBs) were used, the predominant mesenchymal cells that become activated and contribute to the desmoplastic stroma in PDAC or BRCA, respectively^{18, 19}. Glutamine titration experiments revealed a higher dependency of primary PSCs and MFBs compared to NIH-3T3 cells on Gln for growth (FIGS. 8A, 8D). Therefore, “10% Gln” was used for NIH-3T3 cells and “20% Gln” was used for primary PSCs and MFBs as the “low Gln” condition in the following experiments. In 20% Gln, PSCs and MFBs were unable to proliferate and produce collagen I when treated with TGFβ (FIGS. 1B, 8A, 8D, and 8E). In addition, TGFβ could no longer stimulate collagen accumulation in the ECM generated by confluent PSCs and MFBs when the Gln concentration in the medium was reduced to 20% or lower (FIGS. 8C and 8F).

Next, why collagen production in TGFβ-stimulated fibroblasts was impaired when the extracellular glutamine concentration was reduced was sought. Based on a recent report suggesting that glutamine-tRNA is often uncharged when cells are cultured in low amino acid medium²⁰, a quantitative PCR (qPCR)-based method was used to analyze the charging state of tRNAs for several non-essential amino acids (NEAAs) and leucine-tRNA as control. A profound uncharging of glutamine-tRNA was observed in TGFβ but not mock-treated NIH-3T3 cells cultured in low Gln (FIG. 1D). Consistent with tRNA uncharging, fibroblasts stimulated with TGFβ in low Gln displayed phosphorylation of the kinase GCN2 (FIG. 1E), which is auto-phosphorylated on binding to uncharged tRNAs and induces a reduction of bulk translation while at the same time upregulating translation of ATF4 to activate the integrated stress response²¹. Consistently, ATF4 was upregulated in TGFβ-stimulated fibroblasts in low Gln, while bulk translation was reduced compared to control cells (FIGS. 1E and 1F). These data suggest that an inability to maintain Gln tRNA charging could lead to activation of GCN2 and reduced translation in fibroblasts treated with TGFβ in a low Gln environment, resulting in a reduction of ECM synthesis. Gln-tRNA charging has been reported to be restored in proliferative fibroblasts by inhibiting glutaminase with the allosteric inhibitor CB839²⁰. However, CB839 treatment did not rescue GCN2 activation induced by TGFβ treatment in low Gln and only marginally increased collagen I protein levels (FIG. 1G), indicating that other fates of Gln could also be limiting for collagen production.

Glutamine is a major anaplerotic substrate in proliferating cells²² and in TGFβ-stimulated fibroblasts¹¹. Notably, in addition to glutamine, free levels of most TCA cycle intermediates were substantially reduced in fibroblasts treated with TGFβ in low Gln, as were several TCA cycle related NEAAs including glutamate, aspartate and asparagine (FIG. 1H). Asparagine and proline individually did not rescue the TGFβ-induced growth defect and collagen I depletion in low Gln (FIGS. 8G and 8H). However, high levels of cell-permeable glutamate (dm-Glu) but not proline (m-Pro) were sufficient to restore growth and collagen I levels in TGFβ-treated fibroblasts in low Gln (FIGS. 1I and 1J). A cell-permeable version of the TCA cycle intermediate alpha-ketoglutarate (dm-αKG) was also able to rescue both growth and collagen I levels under these conditions (FIGS. 1I and 1J). Dm-Glu and dm-αKG both rescued the TGFβ-induced depletion of TCA cycle intermediates and NEAAs and prevented GCN2 activation in low Gln medium (FIG. 1J and FIG. 8I). PSCs and MFBs cultured in low Gln also showed GCN2 phosphorylation when treated with TGFβ (FIGS. 8J and 8K). Consistently, analysis of tRNA charging under these conditions revealed uncharging of both glutamine and aspartate-tRNA in PSCs (FIG. 8L) and aspartate-tRNA in MFBs (FIG. 8M). The additional uncharging of aspartate-tRNA in PSCs and MFBs treated with TGFβ in low Gln indicated that these cells were further limited in their ability to maintain the TCA cycle to support NEAA synthesis and collagen production. Consistent with this idea, supplementing TGFβ-treated PSCs and MFBs in low Gln with cell-permeable anaplerotic substrates or aspartate rescued collagen levels and GCN2 phosphorylation (FIGS. 8N and 8O).

### Example 2: Glutamine De Novo Synthesis can Maintain Collagen Synthesis

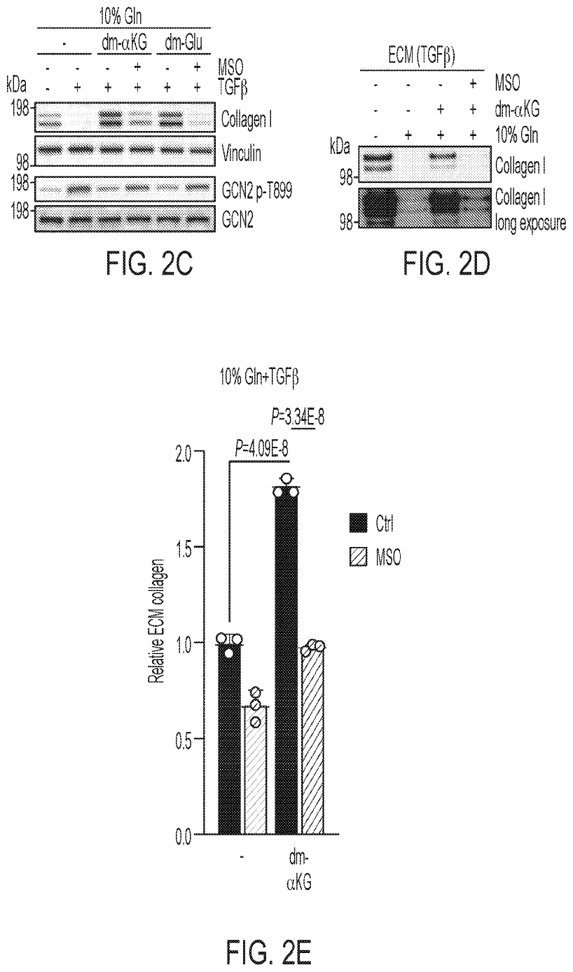

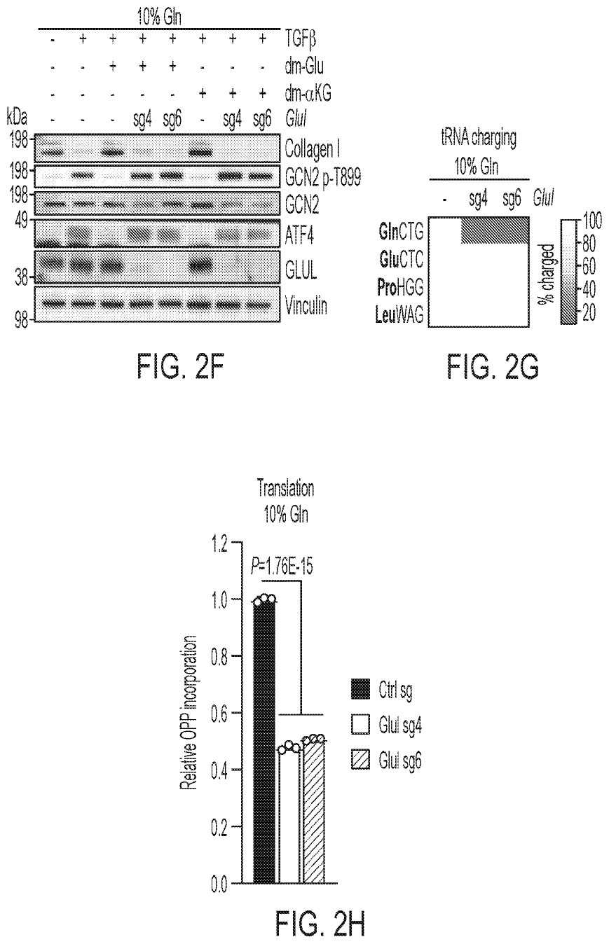

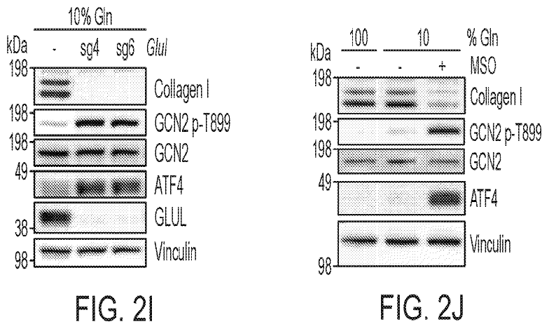

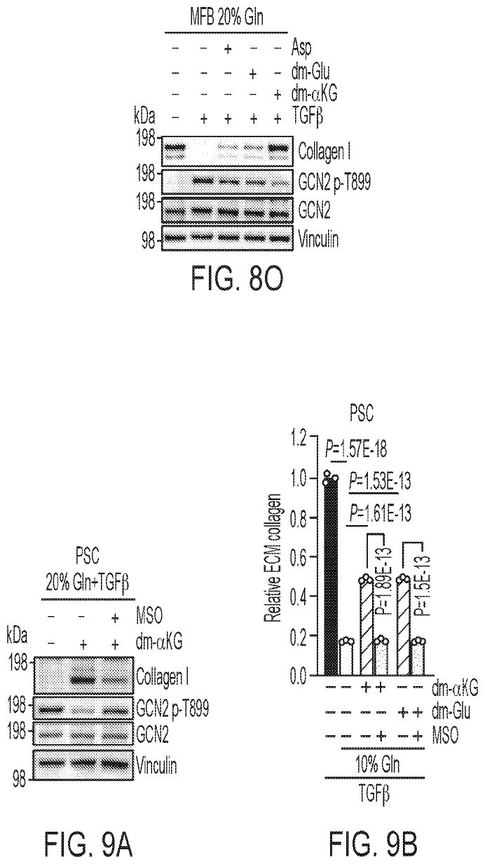

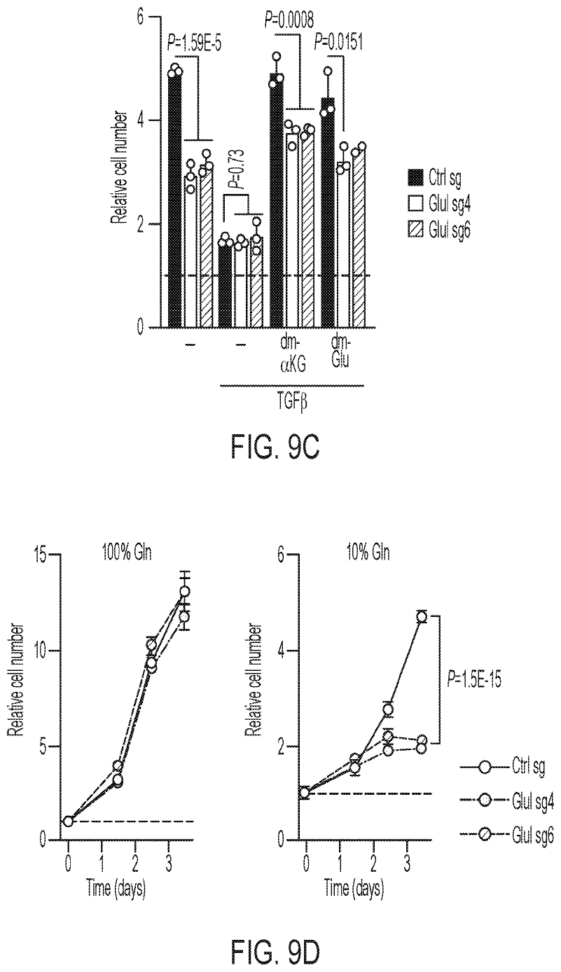

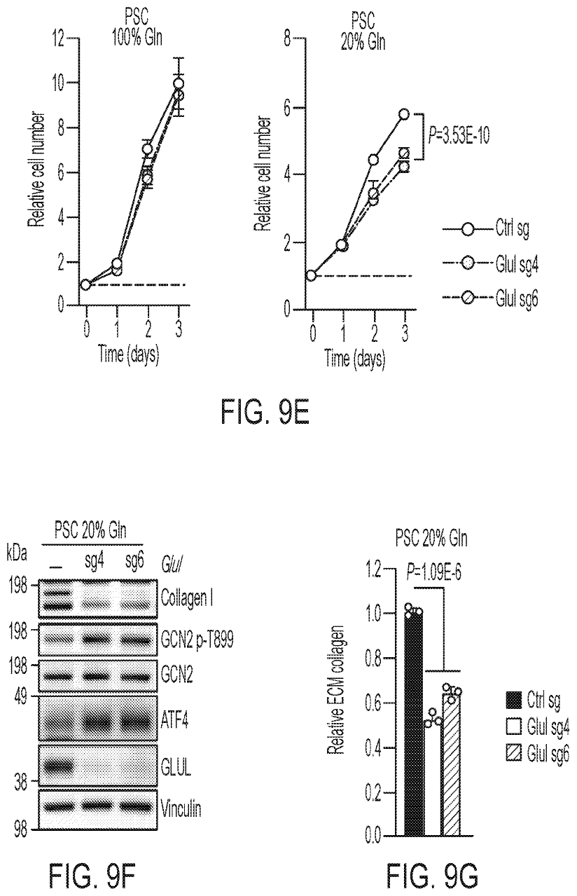

In addition to being used as an anaplerotic substrate, αKG can be transaminated to glutamate, which can then be amidated by glutamine synthetase (encoded by Glul) to synthesize glutamine de novo. Glutamine de novo synthesis has been shown to be active in tumor-associated fibroblasts in ovarian and pancreatic cancer^{23,24}. Given that TGFβ-treated NIH-3T3 cells and PSCs showed glutamine-tRNA uncharging in low Gln, whether dm-αKG supplementation promotes collagen production by supporting glutamine de novo synthesis was sought. Treatment with dm-αKG restored the charging of glutamine-tRNA in TGFβ-treated cells in low Gln, and this was blocked by methionine sulfoximine (MSO), an irreversible inhibitor of GLUL (FIG. 2A). Treatment with dm-αKG also suppressed GCN2 activation and promoted translation in TGFβ-treated cells in low Gln, which was dependent on glutamine de novo synthesis (FIGS. 2B and 2C). In addition, the restoration of collagen levels by dm-Glu and dm-αKG in TGFβ-treated NIH-3T3 cells and PSCs in low glutamine was prevented by treatment with MSO (FIGS. 2C-2E and FIGS. 9A-9B). To confirm these results genetically, Glul was deleted by CRISPR/Cas9. Deletion of Glul compromised fibroblast growth in low Gln in the absence of TGFβ (FIGS. 9C and 9D) and significantly reduced the ability of dm-αKG and dm-Glu to rescue the proliferation of TGFβ-treated cells (FIG. 9C). In addition, Glul deletion almost completely blocked the increase of collagen I protein on dm-αKG and dm-Glu supplementation in TGFβ-treated fibroblasts in low Gln containing medium (FIG. 2F). Glul deletion resulted in glutamine-tRNA uncharging, GCN2 activation and a reduction in protein translation in untreated fibroblasts cultured in low Gln (FIGS. 2G-2I). In addition, NIH-3T3 cells were unable to maintain collagen I synthesis when Glul was deleted or inhibited with MSO (FIGS. 2I and 2J). Similar results were obtained in Glul-deleted PSCs (FIGS. 9E-9G).

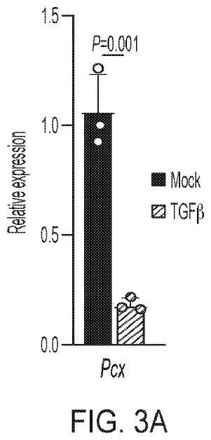

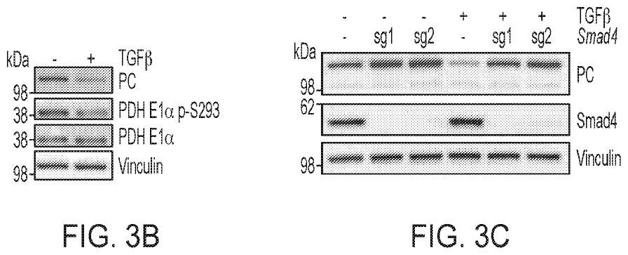

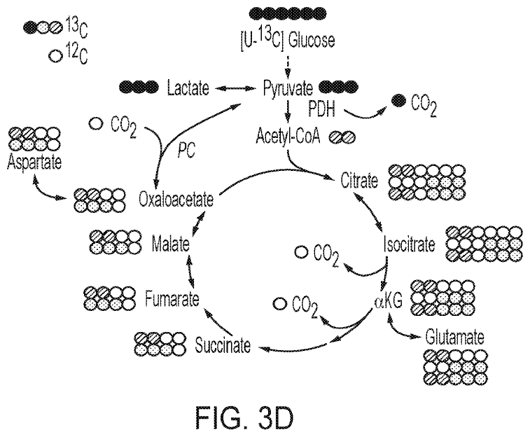

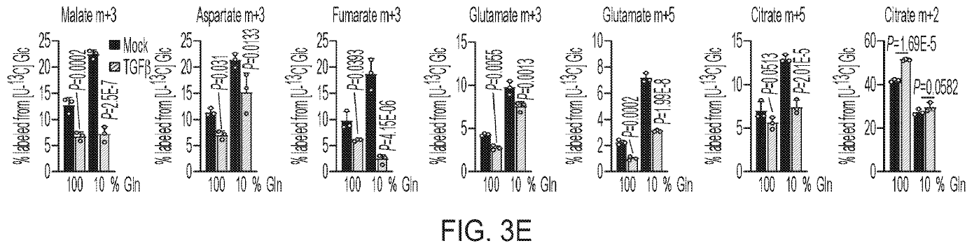

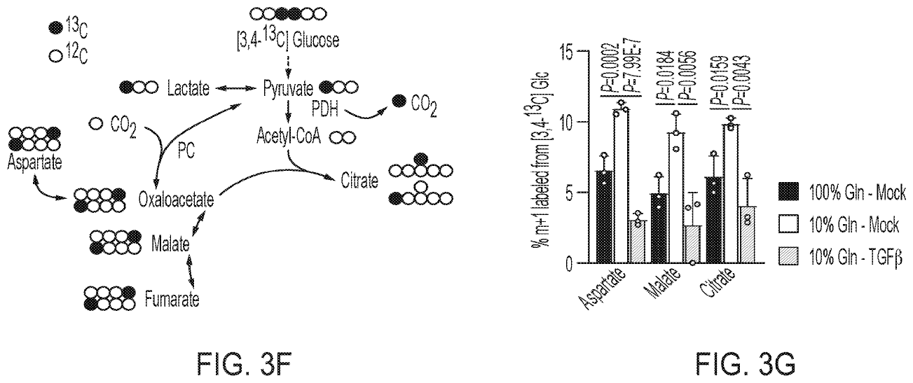

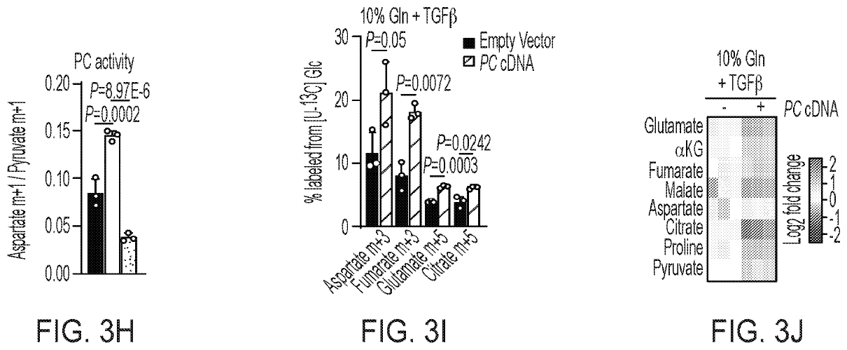

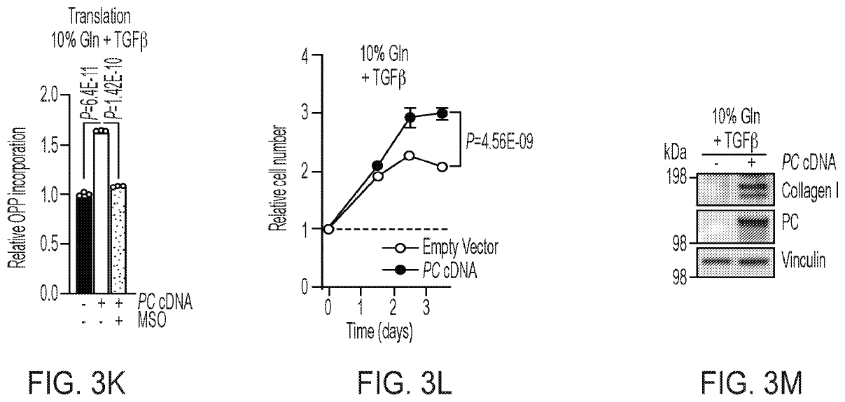

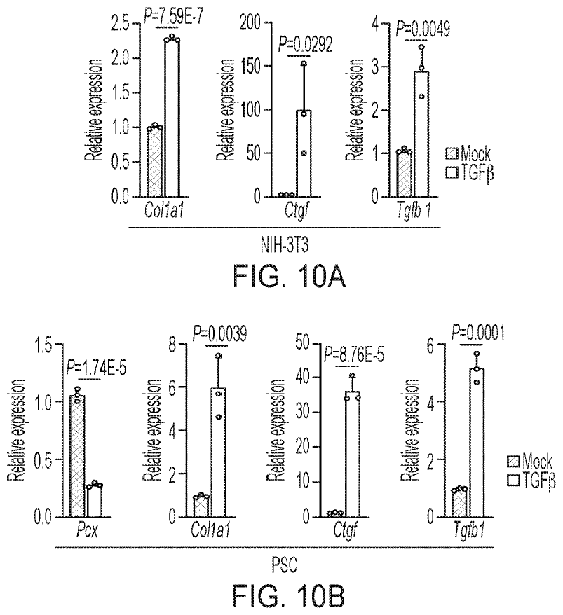

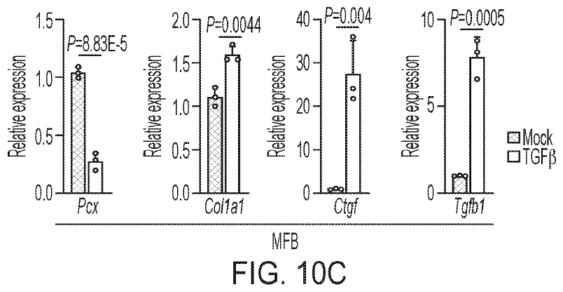

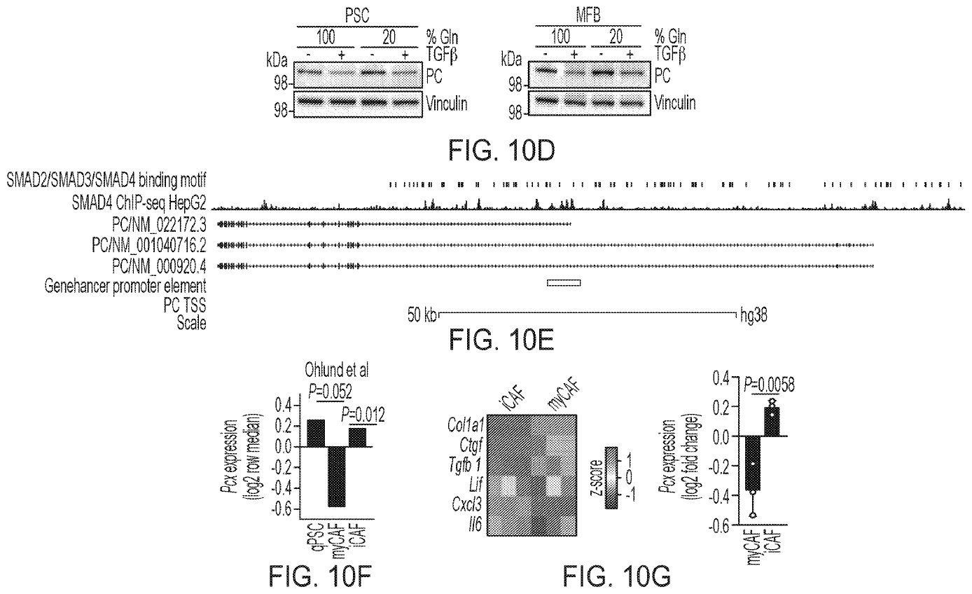

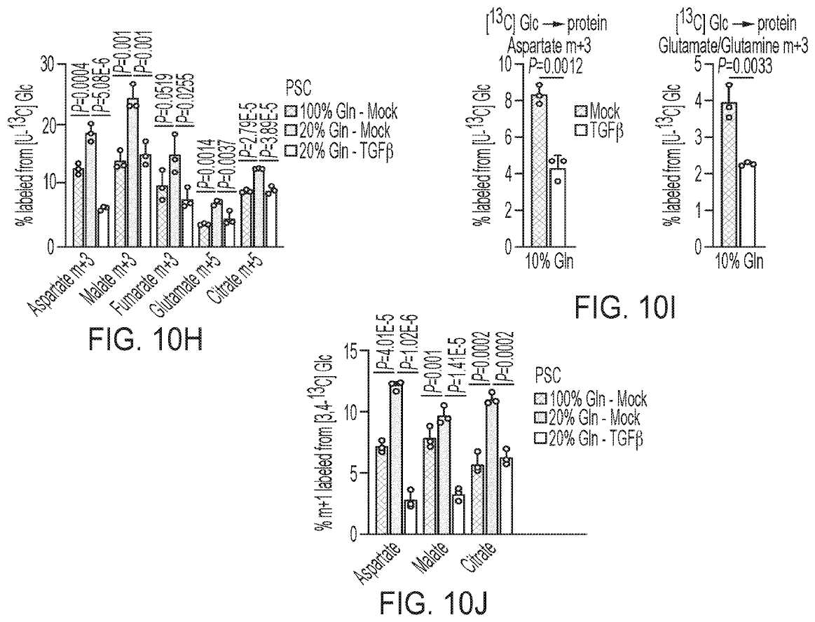

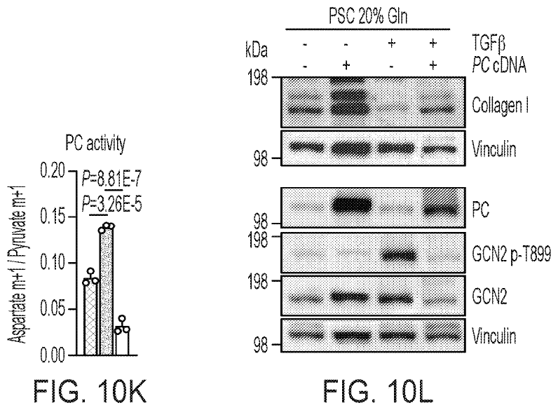

The requirement for GLUL to maintain cell proliferation and collagen production in fibroblasts cultured in low Gln in the presence or absence of TGFβ suggested that TGFβ treatment resulted in a dependency on glutamine for TCA cycle anaplerosis, indicating an inability to use other anaplerotic substrates. The other main anaplerotic substrate is pyruvate which can be converted to oxaloacetate by PC. Some cancer cells can maintain growth via PC-mediated anaplerosis from pyruvate when glutamine-derived anaplerosis is limited in vitro, or in the tumor microenvironment in vivo²⁵⁻²⁸. To see if fibroblasts also used PC, PC messenger RNA (Pcx in mouse) and protein expression was analysed first. PC transcript and protein levels were reduced by TGFβ treatment in all fibroblast types analysed, despite other well-known TGFβ-responsive genes being induced (FIGS. 3A-3B and FIGS. 10A-10D). TGFβ treatment also reduced the repressive phosphorylation of the E1α subunit of the pyruvate dehydrogenase (PDH) complex, indicative of higher activity (FIG. 3B). To test whether the reduced expression of PC could be a direct effect of TGFβ signaling, binding sites for the TGFβ-induced transcriptional mediators SMAD2/3/4 were examined around the putative promoter region and transcriptional start site (TSS) of human PC. Indeed, SMAD2/3/4 binding motifs were enriched within the analysed regions (P<0.0001, FIG. 10E). In addition, analysis of human SMAD4 chromatin-immunoprecipitation (ChIP) sequencing data from the ENCODE project confirmed enrichment of SMAD4 at the putative PC promoter region and TSS (FIG. 10E). Through CRISPR/Cas9-mediated deletion of Smad4, it was confirmed that TGFβ-induced downregulation of PC protein was Smad4-dependent (FIG. 3C). PC expression was also analysed in different subtypes of cancer-associated fibroblasts (CAFs) that have been described in PDAC²⁹, which revealed lower PC mRNA expression in TGFβ-driven myofibroblastic CAFs (myCAFs) compared to interleukin-1 (IL1)-driven inflammatory CAFs (iCAFs) (FIGS. 10F and 10G).

### Example 3: PC Activity is Suppressed by TGFβ

To test whether TGFβ treatment also alters PC activity, the fate of fully [¹³C]-labelled glucose ([U-¹³C]Glc) into TCA cycle intermediates and related NEAAs was traced, focusing on three carbon labelling (m+3) which can be used as a surrogate for PC activity (FIG. 3D). Culture of control fibroblasts in low Gln increased m+3 labelling of TCA cycle intermediates and NEAAs from [U-¹³C]Glc (FIG. 3E), indicating an increase in the relative contribution of PC to the pool of these metabolites. In addition, m+5 labelled glutamate and citrate, reflective of the combined activity of PC and PDH, were increased on culture in low Gln (FIG. 3E). In contrast, TGFβ treatment reduced m+3 and/or m+5 labelling of the metabolites analysed, in both high and low Gln containing medium, while m+2 labelling of citrate, indicative of PDH activity, was increased (FIG. 3E), consistent with reduced PDH phosphorylation in TGFβ-treated cells (FIG. 3B). Similar results were obtained in PSCs (FIG. 10H). In addition, TGFβ reduced m+3 labelling of aspartate and glutamate/glutamine that are incorporated into cellular protein (FIG. 10I). To support these findings further, tracing experiments were performed with [3,4-¹³C]Glc. This tracer is metabolized to [1-¹³C]pyruvate and yields one-carbon labelled TCA cycle intermediates when carboxylated via PC, while the label is lost when decarboxylated via PDH (FIG. 3F)²⁵. Consistent with the above results, m+1 labelling of aspartate, malate and citrate increased when fibroblasts were cultured in low Gln, and this increase was suppressed by TGFβ treatment, indicative of lower PC activity (FIGS. 3G and 3H). Similarly, PC activity increased in PSCs cultured in low Gln but was suppressed in the presence of TGFβ (FIGS. 10J and 10K).

To confirm the influence of PC in the inability of TGFβ-stimulated fibroblasts to grow or produce collagen when cultured in low Gln containing medium, a PC cDNA was introduced in NIH-3T3 cells and PSCs. Both the absolute levels, and the m+3 and m+5 labelling of TCA cycle intermediates and NEAAs from [U-¹³C]Glc was increased in cells expressing PC cDNA growing in 10% Gln in the presence of TGFβ (FIGS. 31 and 3J). PC overexpression increased protein translation in TGFβ-treated cells cultured in low Gln, and this increase was blocked by the GLUL inhibitor MSO (FIG. 3K), indicating that PC-supported protein translation depends on glutamine de novo synthesis. Expression of PC cDNA was also sufficient to increase cell growth and collagen I protein when TGFβ-treated cells were cultured in low glutamine (FIGS. 3L-3M and FIG. 10L).

### Example 4: PC Anaplerosis is Required for Collagen Synthesis in Low Gin

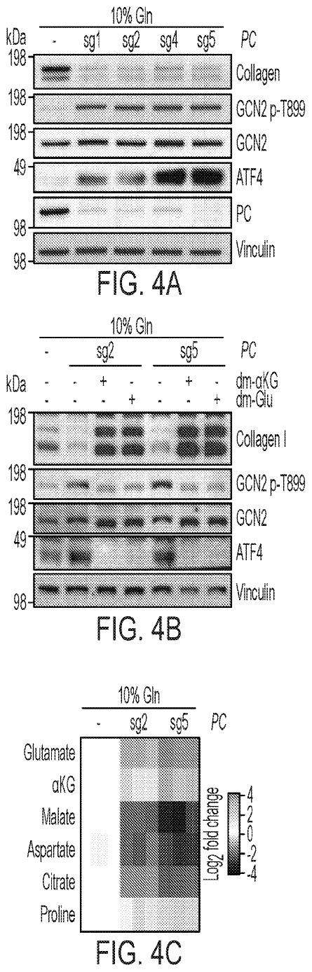

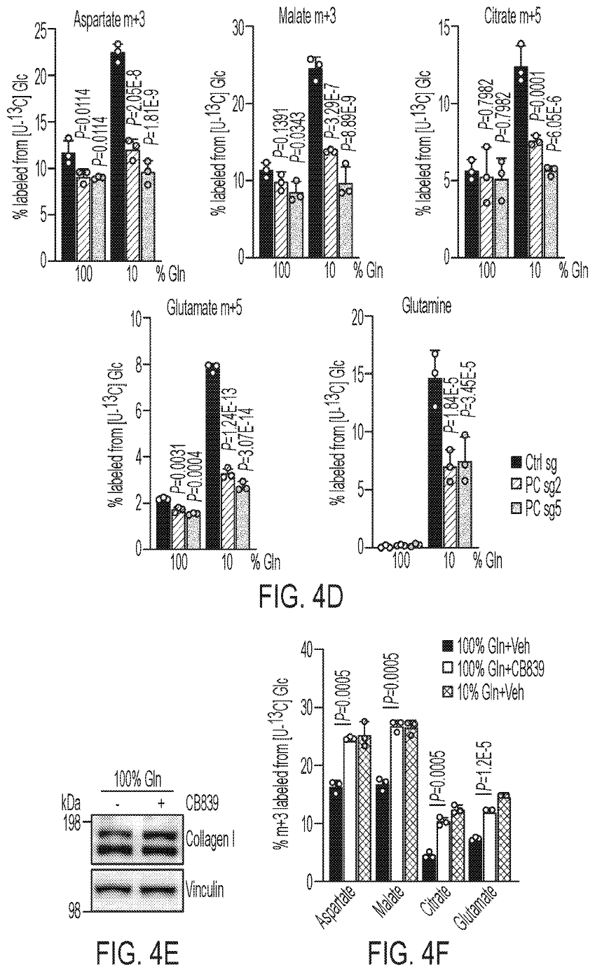

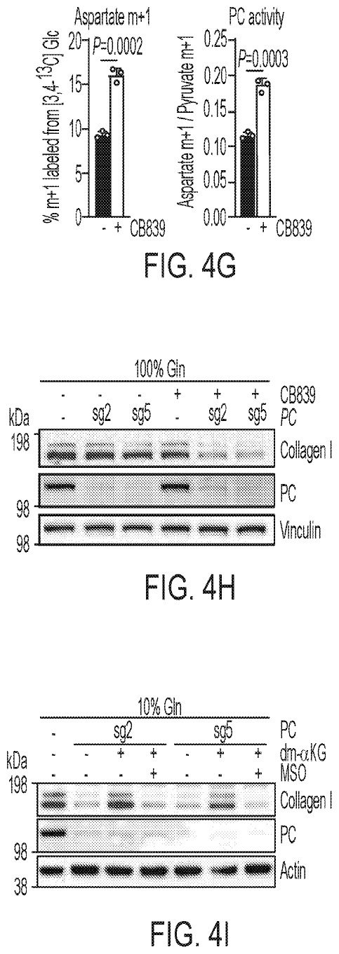

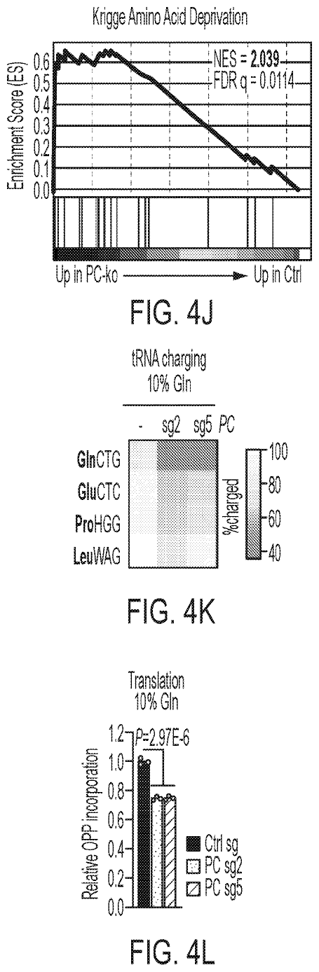

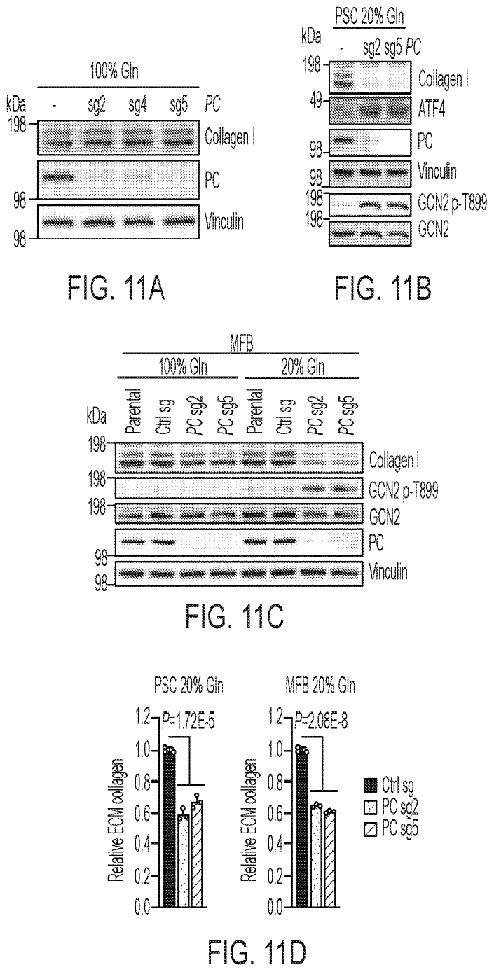

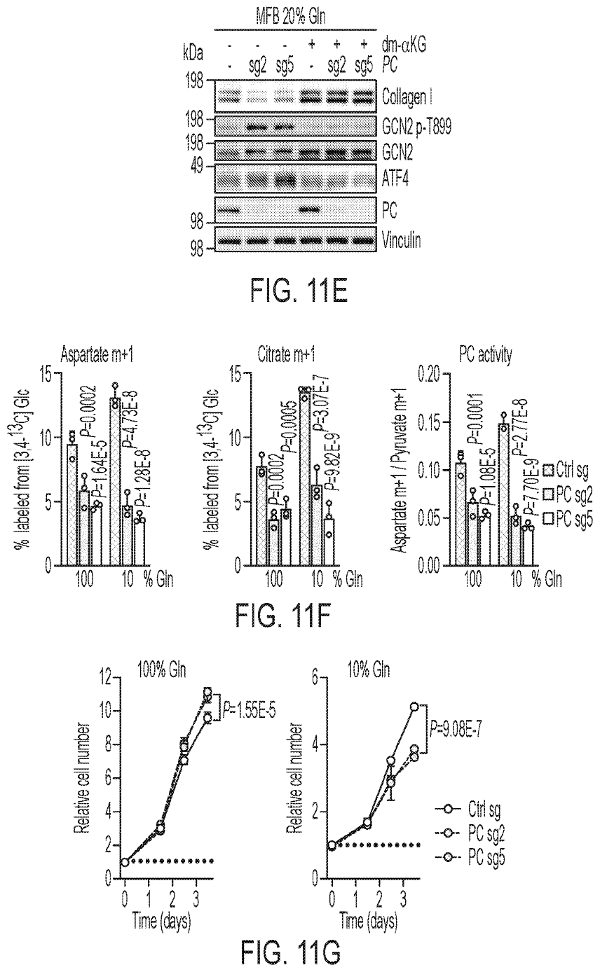

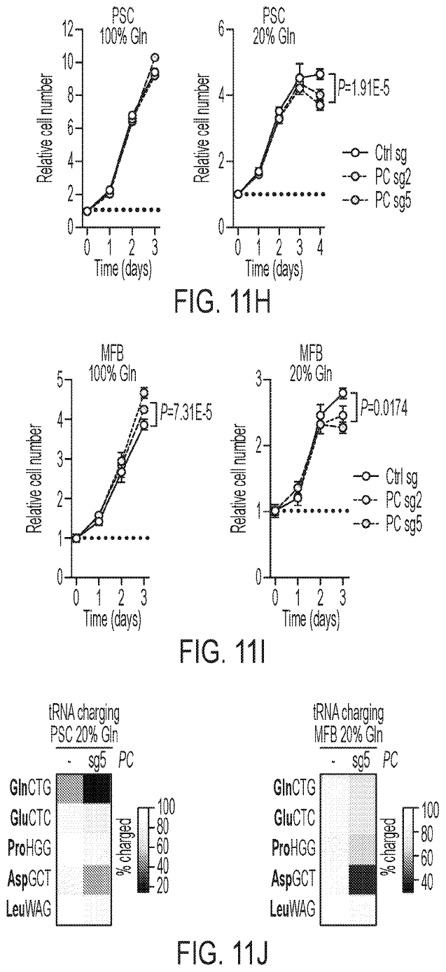

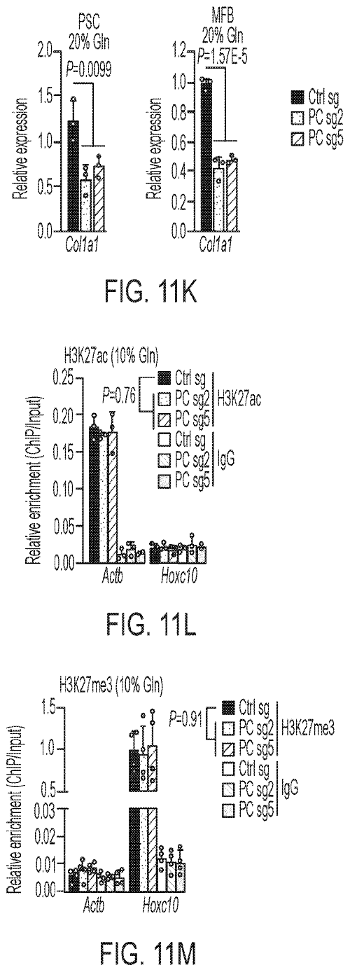

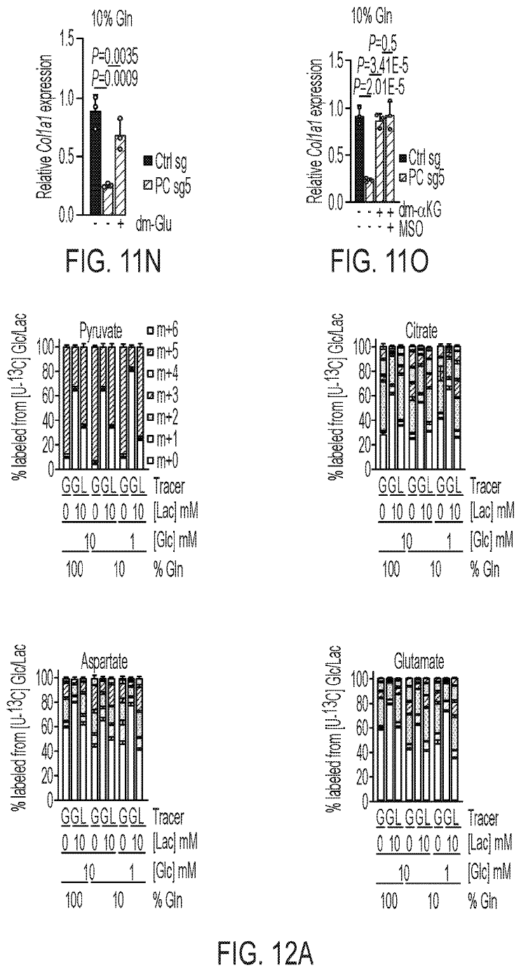

In the absence of TGFβ , fibroblasts grown in low Gln medium were able to maintain collagen production (FIG. 1C). Next, whether collagen synthesis under these conditions required PC was sought. Fibroblasts grown in low Gln were unable to accumulate collagen I when PC was deleted, and these cells had increased GCN2 phosphorylation that was accompanied by accumulation of ATF4 (FIG. 4A). In contrast, there was no change in collagen I accumulation in comparison to wild-type cells when PC-deleted cells were cultured in high Gln (FIG. 11A). Similar results were obtained when PC was deleted in PSCs and MFBs (FIGS. 11B and 11C). Importantly, reduced levels of collagen were also observed in the ECM produced by confluent PSCs and MFBs with PC deletion cultured in low Gln (FIG. 11D). To test whether the inability of PC-deleted cells to synthesize collagen I was due to the lack of anaplerotic input, these cells were supplemented with dm-αKG or dm-Glu. Both dm-αKG and dm-Glu increased collagen I levels in PC-deleted cells cultured in low Gln (FIG. 4B and FIG. 11E). Indeed, TCA cycle intermediates and related NEAAs were depleted in PC-deleted cells cultured in low glutamine (FIG. 4C). To confirm that PC deletion impairs TCA cycle anaplerosis from glycolytic carbon, glucose metabolic tracing studies were performed comparing control and PC-deleted cells. Control fibroblasts grown in low Gln increased their m+3 and m+5 labelling of TCA cycle intermediates and related NEAAs from [U-¹³C]Glc compared to cells grown in high Gln (FIG. 3E and FIG. 4D), and this increase was suppressed when PC was deleted (FIG. 4D). Furthermore, PC deletion reduced m+1 labelling of aspartate and citrate from [3,4-¹³C]Glc in both high and low Gln (FIG. 11F). While some pyruvate carboxylation activity remained in PC-deleted cells, possibly due to the activity of malic enzyme²⁷, only PC-dependent pyruvate carboxylation increased on culture in low Gln (FIG. 11F).

In high Gln, PC deletion if anything promoted cell growth, while in low Gln there was a reduction in cell growth on PC deletion (FIG. 11G). Similar results were obtained in PSCs and MFBs (FIGS. 11H and 11I). To support these findings further, glutaminase inhibitor CB839 was used to block TCA cycle anaplerosis from glutamine. CB839 treatment alone did not affect collagen I levels in cells cultured in full medium (FIG. 4E), but increased m+3 labelling of TCA cycle intermediates and related NEAAs from [U-¹³C]Glc (FIG. 4F), indicative of increased anaplerosis via PC. Consistent with this, m+1 labelling of aspartate and pyruvate carboxylation activity from [3,4-13C]Glc was increased in cells treated with CB839 (FIG. 4G). To test whether PC-mediated anaplerosis supports collagen synthesis when anaplerosis from glutamine is inhibited, PC-deleted cells were treated with CB839. In the absence of PC, CB839 treatment resulted in reduced collagen I levels (FIG. 4H). Thus, PC-mediated TCA cycle anaplerosis supports collagen production when anaplerosis from glutamine is impaired.

Glucose metabolic tracing studies have indicated that PC-deleted cells are impaired in their ability to use glucose-derived carbon for TCA cycle anaplerosis and the synthesis of NEAAs including glutamine (FIG. 4D and FIG. 11F). This suggested that PC-mediated anaplerosis could maintain collagen I levels at least in part by supporting glutamine de novo synthesis. To test this, PC-deleted cells cultured in low Gln and supplemented with dm-αKG were treated with the GLUL inhibitor MSO. In the presence of MSO, dm-αKG was unable to restore collagen I levels in cells with PC deletion (FIG. 4I). Furthermore, PC-deleted cells cultured in low Gln medium were enriched for a gene expression signature characteristic of amino acid deprived cells (FIG. 4J), which was consistent with a selective uncharging of glutamine and/or aspartate-tRNA (FIG. 4K and FIG. 11J) and a reduced translation rate (FIG. 4L).

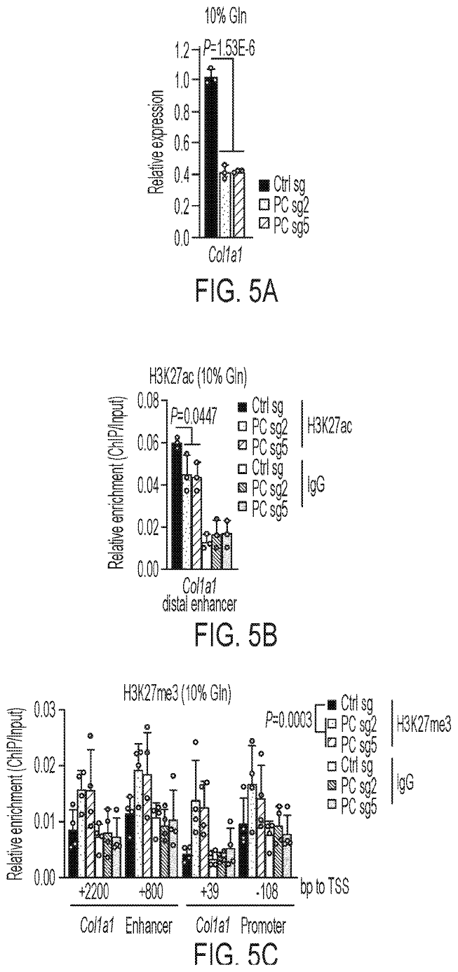

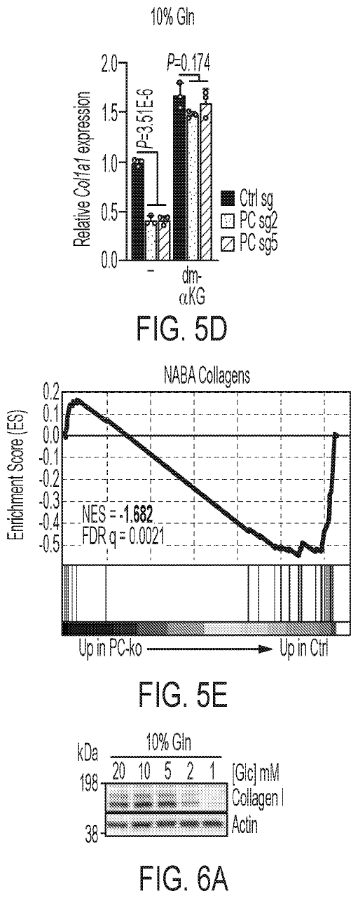

As loss of PC resulted in changes in gene expression on culture in low Gln, whether the impairment in collagen synthesis was also present at the transcriptional level was sought. Indeed, Col1a1 mRNA was downregulated in PC-deleted cells cultured in low Gln (FIG. 5A). This effect was also observed in PSCs and MFBs (FIG. 11K). Changes in metabolite availability can influence gene expression by impacting the deposition and removal of chromatin modifications³⁰. Histone acetylation is associated with actively transcribed genes and is controlled in part by the availability of citrate-derived acetyl-CoA. Reduced citrate levels in PC-deleted cells cultured in low Gln (FIG. 4C) suggested that histone acetylation could be impaired in these cells. To test this, ChIP-qPCR was performed, which showed that levels of acetylated H3K27 (H3K27ac), a mark associated with active enhancers, were significantly reduced at the distal Col1a1 enhancer in PC-deleted cells cultured in low Gln, but not at control loci (FIG. 5B and FIG. 11L). H3K27 can either be acetylated or methylated, and consistent with reduced H3K27ac, the repressive trimethylated H3K27 (H3K27me3) histone modification was enriched across two enhancer and two promoter regions of the Col1a1 locus in PC-deleted cells cultured in low Gln, but not at control loci (FIG. 5C and FIG. 11M). Based on these results, it was hypothesized that the addition of an alternative anaplerotic substrate would promote Col1a1 mRNA expression when PC is lost. Indeed, supplementation with dm-αKG or dm-Glu rescued Col1a1 mRNA expression in PC-deleted cells cultured in low Gln (FIG. 5D and FIG. 11N), which was independent of glutamine de novo synthesis (FIG. 11O). The expression of most collagen genes was downregulated in PC-deleted cells compared to control cells cultured in low Gln containing medium (FIG. 5E), indicating a general repression of collagen transcription when PC is lost.

### Example 5: Lactate Supports Collagen Synthesis Via PC in Low Glc/Gln

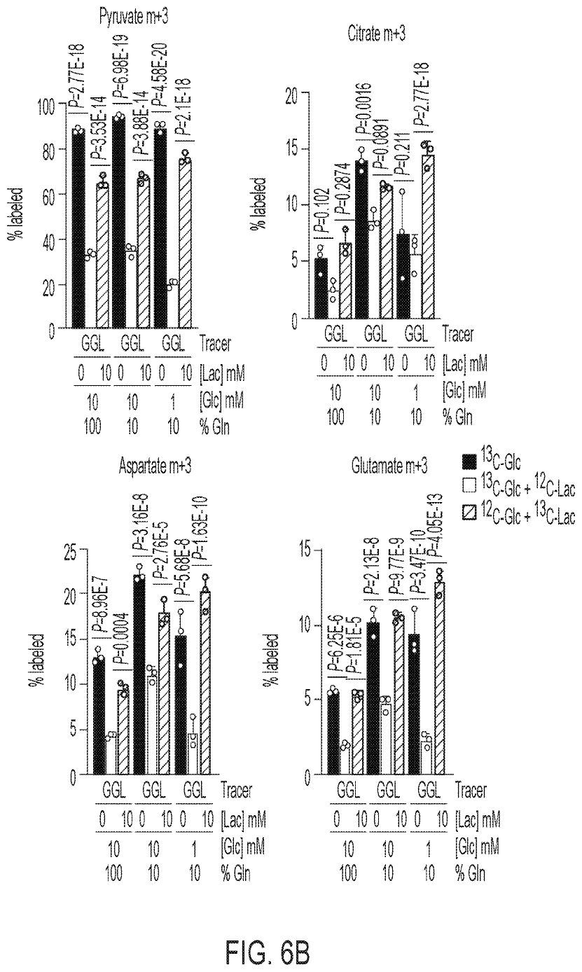

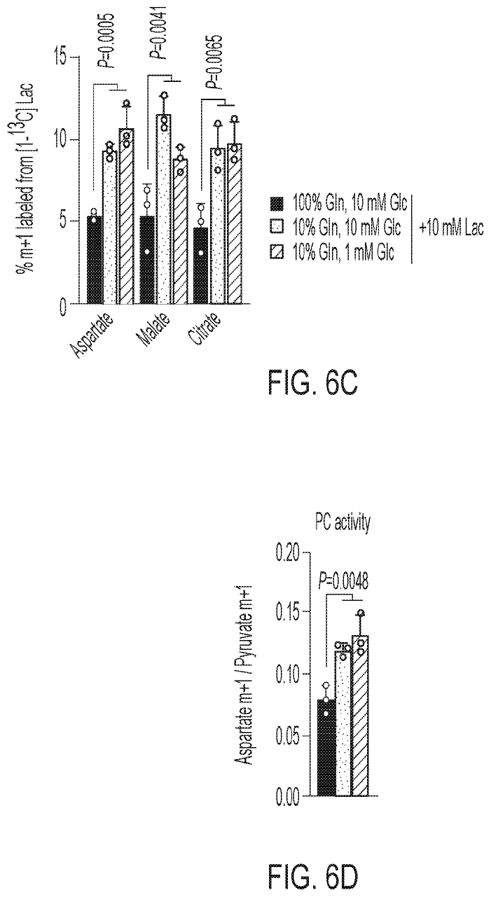

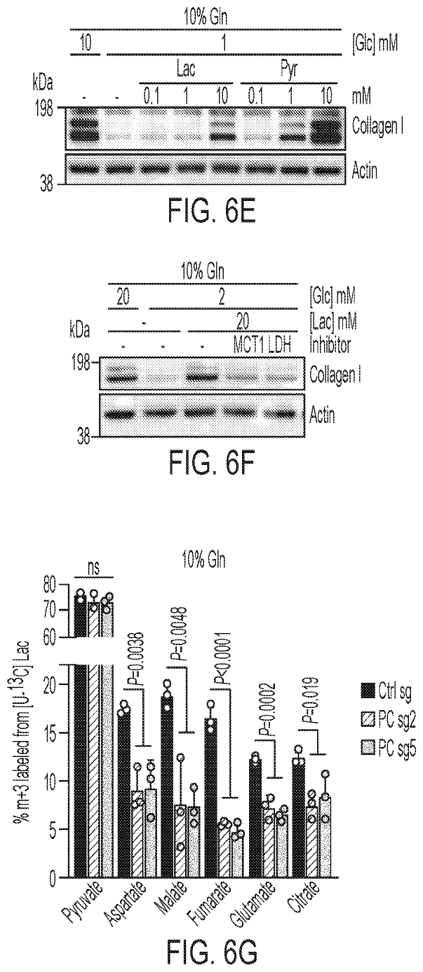

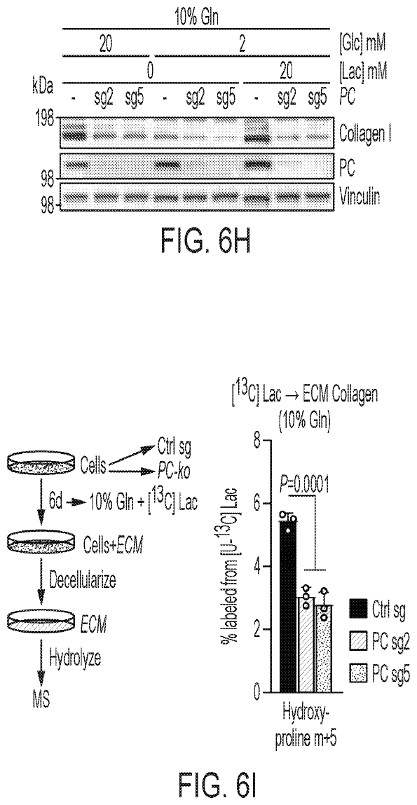

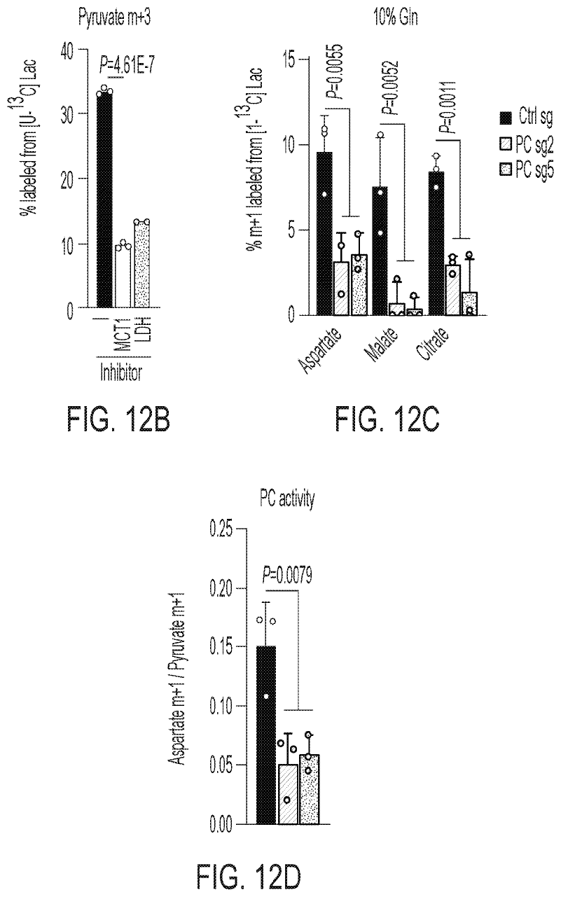

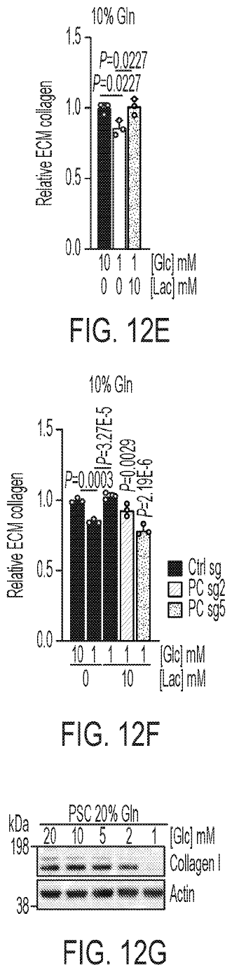

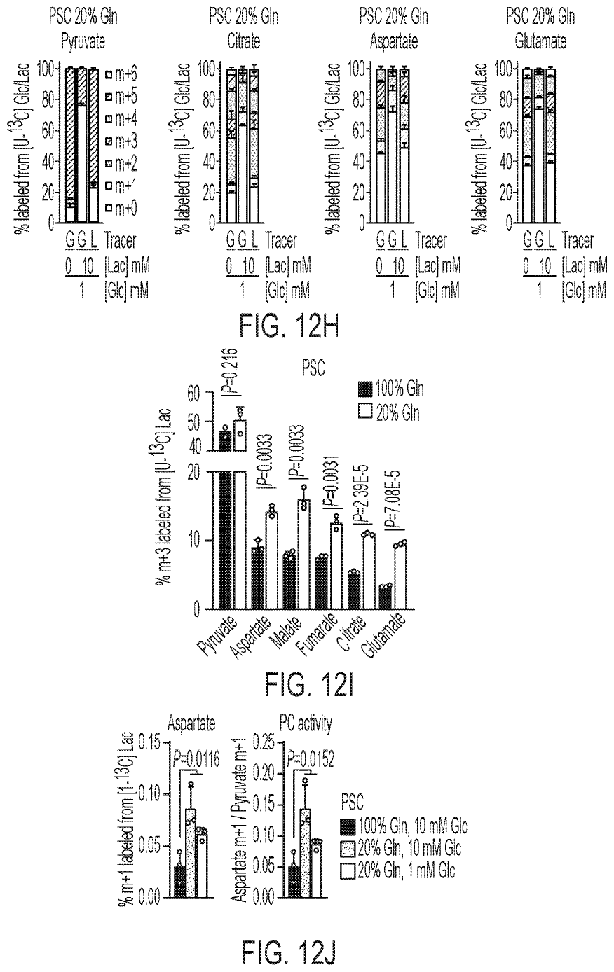

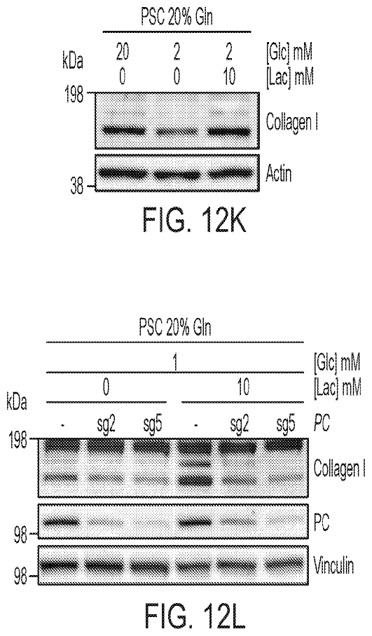

The above data demonstrate that when extracellular glutamine concentrations are low, collagen production in fibroblasts can be maintained by PC-mediated anaplerosis from glycolytic carbon. However, glucose levels are also often reduced in tumors^{15, 17}, indicating that glucose-derived pyruvate for PC-mediated TCA cycle anaplerosis could be limiting in vivo. Indeed, fibroblast production of collagen I progressively declined when the concentration of extracellular glucose was reduced in low Gln containing medium (FIG. 6A and FIG. 12G), raising the question of how fibroblasts support TCA cycle anaplerosis for collagen synthesis in vivo.

While glutamine and glucose availability can be limited in tumors, lactate is the most consistently elevated metabolite in human tumors³¹. To test whether fibroblasts can use lactate for TCA cycle anaplerosis, metabolic tracing studies were performed with uniformly labelled lactate ([U-¹³C]Lac). Consistent with previous studies in ovarian tumor-associated fibroblasts²³, in the presence of extracellular lactate, the contribution of glucose to the cellular pyruvate pool was reduced, and the majority of pyruvate was derived from extracellular lactate, even when extracellular glucose and glutamine were abundant (FIG. 6B and FIG. 12A). In fibroblasts supplemented with lactate, lactate-derived pyruvate preferentially contributed to the TCA cycle and related NEAAs compared to glucose-derived pyruvate (FIG. 12A), which is consistent with metabolic flux analysis in whole organs³². The utilization of lactate-derived pyruvate via PC (m+3) caused an increase in low Gln containing medium (FIG. 6B), indicating that lactate-derived pyruvate could act as anaplerotic substrate. Consistent with this, m+1 labelling of aspartate, malate and citrate and pyruvate carboxylation activity from [1-¹³C]Lac was elevated under low Gln culture conditions (FIGS. 6C-6D).

Therefore, it was asked whether extracellular lactate could support collagen production when both glutamine and glucose concentrations were reduced. Indeed, lactate supplementation to fibroblasts cultured in low glutamine and low glucose increased collagen 1 levels (FIG. 6E). Pyruvate was more potent than lactate; however, at concentrations relevant in vivo (10 mM lactate, 0.1 mM pyruvate), only lactate supplementation increased collagen 1 levels under low glutamine and low glucose conditions (FIG. 6E). This effect was dependent on lactate import via MCT1 and conversion to pyruvate via LDH (FIG. 6F and FIG. 12B). To test whether lactate supported TCA cycle anaplerosis via PC, [U-¹³C]Lac tracing was performed in cells with PC deletion cultured in low glutamine. While there was no difference in pyruvate labelling from lactate in PC-deleted cells, lactate carbon could no longer be used for TCA cycle anaplerosis in the absence of PC (FIG. 6G). This was confirmed using [1-¹³C]Lac as a tracer (FIGS. 12C and 12D). Consistent with these results, the ability of extracellular lactate to rescue collagen I in cells cultured in low glutamine and low glucose was PC-dependent (FIG. 6H). In addition, lactate increased collagen levels in the ECM produced by confluent fibroblasts in low glutamine and low glucose containing medium in a PC-dependent fashion (FIGS. 12E and 12F). Tracing of [U-¹³C]Lac into hydrolyzed ECM proteins revealed that lactate carbon directly contributed to collagen via PC (FIG. 6I). Similar results were obtained in PSCs (FIGS. 12G-12L).

### Example 6: Fibroblast PC supports tumor fibrosis and growth

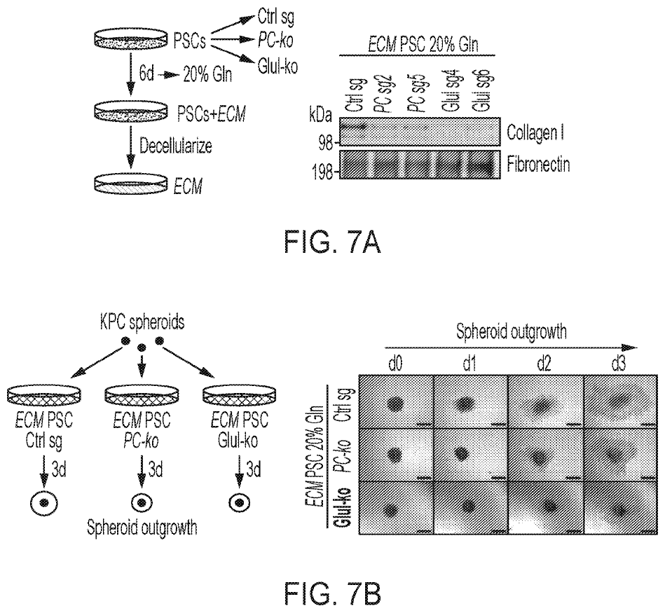

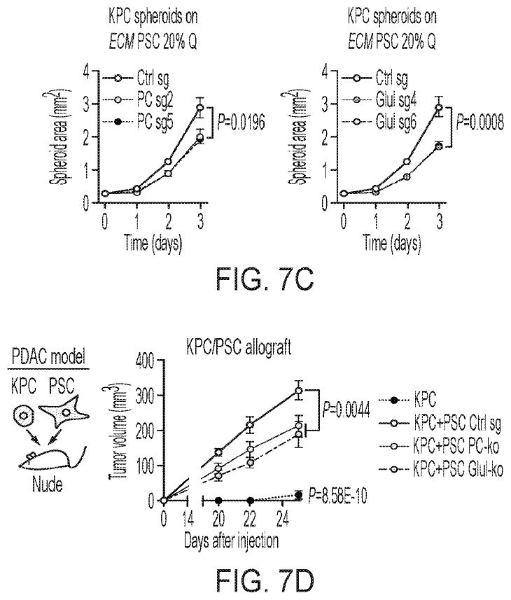

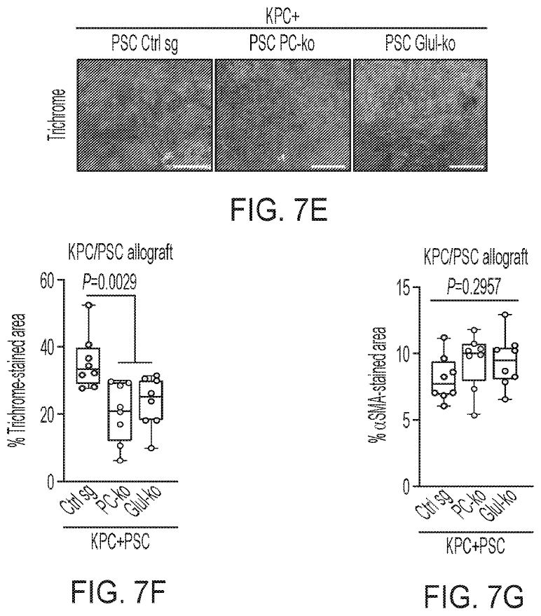

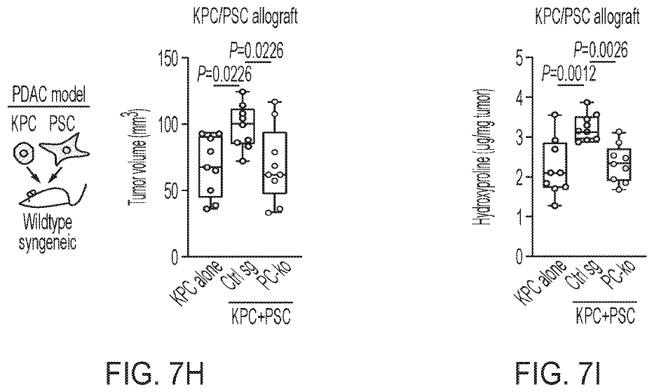

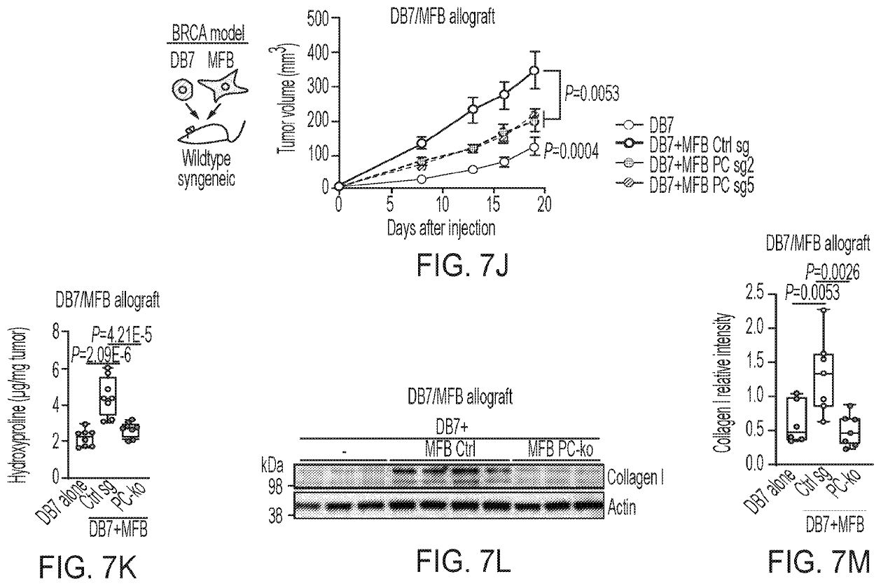

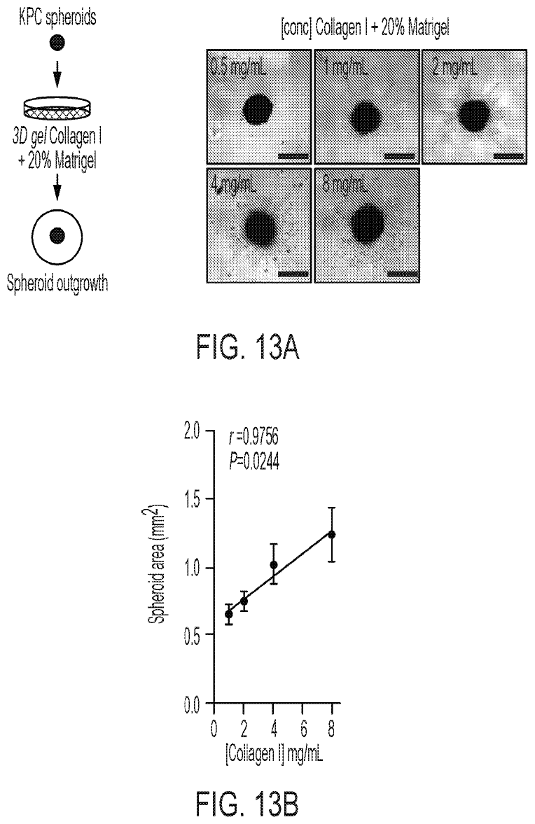

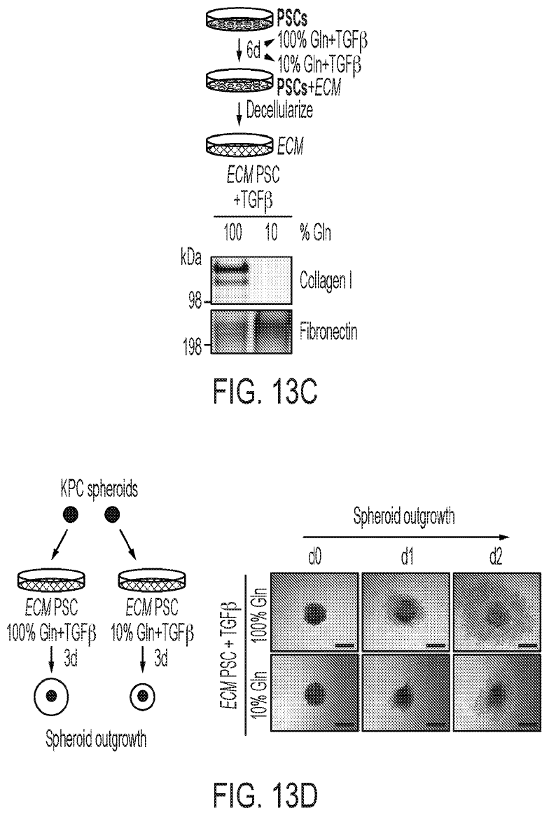

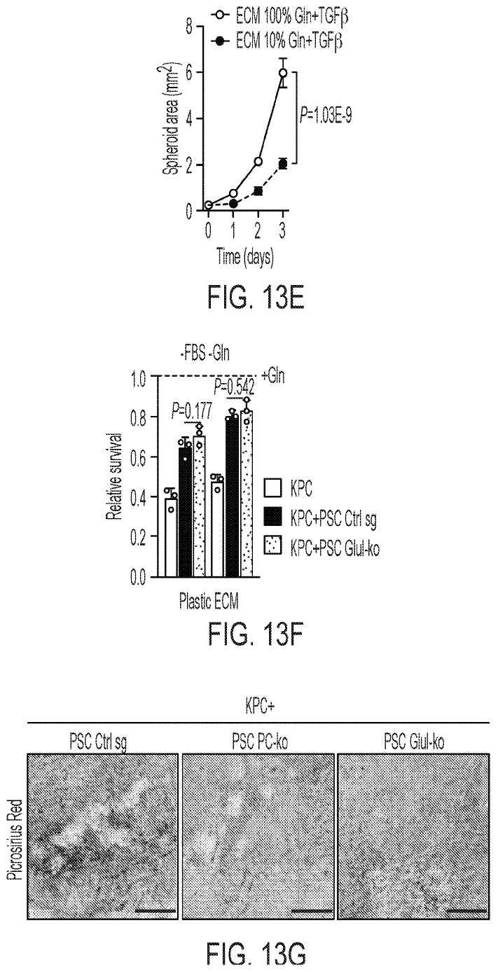

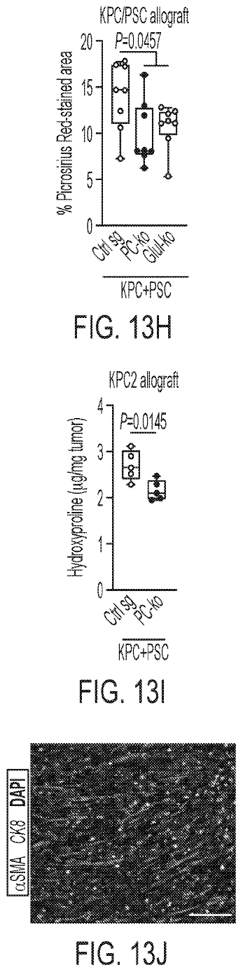

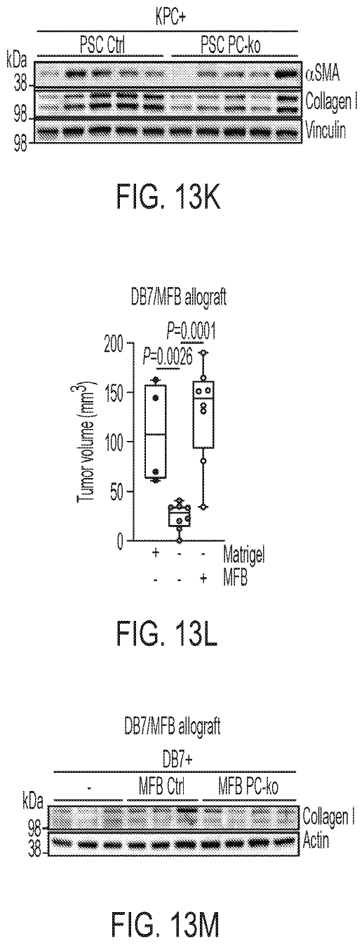

Having established that PC-mediated anaplerosis supports collagen production in fibroblasts under conditions reminiscent of a nutrient-poor tumor microenvironment, how PC-regulated collagen levels in fibroblast-derived ECM affect cancer cells was sought. To this end, a three-dimensional (3D) spheroid culture system was used, in which KPC mouse PDAC cells were first plated on ultra-low attachment plates to form spheroids that were then transferred onto ECM on which cells start to grow out of the spheroid and proliferate. First, cultured KPC spheroids were cultured on a synthetic ECM generated by gelating increasing concentrations of collagen I with Matrigel, a basement membrane mix containing a variety of matrix proteins (FIG. 13A). Spheroid outgrowth correlated with the concentration of collagen I in the synthetic ECM (FIG. 13B). Next, KPC spheroids were cultured on PSC-derived ECM that was produced in the presence of TGFβ and media containing 100% or 10% Gln (FIG. 13C). ECM produced in 10% Gln by TGFβ-stimulated PSCs was depleted of collagen I but not of fibronectin (FIG. 13C), another prominent ECM protein. Spheroid outgrowth was significantly reduced when cultured on ECM that was produced by TGFβ-treated PSCs in 10% Gln compared to 100% Gln (FIGS. 13D and 13E). Then, untreated PSCs with deletion of PC or Glul were used to prepare ECM in the presence of 20% Gln (FIG. 7A). ECM generated by PC or Glul-deleted PSCs under these conditions had a substantially lower collagen I content compared to control cells but was similarly enriched in fibronectin (FIG. 7A). When KPC spheroids were cultured on ECM generated by PC or Glul-deleted PSCs under 20% Gln, their outgrowth was reduced compared to the outgrowth on ECM produced by control PSCs under these conditions (FIGS. 7B and 7C). It has been reported that fibroblast-derived glutamine can support the survival of PDAC cells under glutamine limitation;²⁴ however, both control and Glul-deleted PSCs were similarly able to promote survival of KPC cells in the absence of extracellular glutamine (FIG. 13F).

Next, whether fibroblast PC and Glul are relevant for collagen levels and tumor growth in vivo was sought. To this end, KPC cells were injected s.c. into the flanks of nude mice, either alone or along with PSCs expressing either a control, PC or Glul sgRNA (FIG. 7D). The presence of PSCs promoted tumor growth substantially (FIG. 6D), as previously reported¹⁹. While PC or Glul-deleted PSCs retained the ability to enhance the growth of KPC-derived tumors, tumor growth was significantly reduced compared to co-injection with control PSCs (FIG. 7D). Intratumoral fibrosis was lower in tumors formed by KPC cells that were co-injected with PC or Glul-deleted PSCs compared to control PSCs (FIGS. 7E-7F and FIGS. 13G-13I), while the levels of αSMA, a marker for activated fibroblasts, were similar across tumors (FIG. 7G and FIGS. 13J-13K). Co-injection of PSCs also promoted the growth and increased the collagen content of KPC-derived tumors in immunocompetent, syngeneic mice in a PC-dependent fashion (FIGS. 7H-7I).

Then, the ability of fibroblast-PC to regulate tumor growth and collagen content in a syngeneic BRCA co-injection model (FIG. 7J) was assessed. Co-injection of MFBs promoted tumor growth of DB7 breast cancer cells in wild-type mice (FIG. 7J), and deletion of PC in MFBs with two different sgRNAs significantly reduced the growth of co-injected tumors (FIG. 7J). The beneficial effect of MFBs on tumor growth was prominent at early time points after co-injection and was similar to the tumor-promoting effect of Matrigel (FIG. 13L). This raised the possibility that the growth of DB7 tumors could be supported by the matrix proteins secreted by MFBs. Consistent with this, co-injection of MFBs substantially increased the collagen content of DB7 allograft tumors after engraftment (FIGS. 7K-7M and FIG. 13M). PC deletion in MFBs resulted in a more than 50% reduction of tumor collagen levels compared to co-injection of control MFBs (FIGS. 7K-7M). Thus, fibroblast PC is required for collagen production in the tumor microenvironment.

### Example 7: Discussion

The ability of fibroblasts to synthesize ECM is critical for wound healing. Fibroblast ECM synthesis can also be coopted by cancer cells to support tumor cell growth in solid tumors such as BRCA and PDAC. In such tumors, the chronic activation of fibroblasts to produce excessive amounts of ECM can modulate many of the hallmarks of cancer⁷. As described herein, an understanding of how fibroblasts maintain ECM production under nutrient-poor conditions present in the tumor microenvironment was sought. The results described herein demonstrate that PC-mediated TCA cycle anaplerosis is a critical regulator of ECM production in tumors.

PC is a widely expressed mitochondrial enzyme that catalyzers the carboxylation of pyruvate to oxaloacetate, and as such pro-vides a mechanism to replenish TCA cycle intermediates that are being consumed in support of macromolecular synthesis³³. PC has been well studied in the liver, where its activity is critical for gluconeogenesis, the urea cycle and antioxidant capacity^{34, 35}. While PC appears to be dispensable for normal and cancer cell growth under standard culture conditions, PC has also been shown to be required to support cancer cell growth in vivo²⁶⁻²⁸. It has been suggested that PC is also critical to the stromal cells in the tumor microenvironment and to their role in supporting cancer cell growth.

It was found that PC-mediated TCA cycle anaplerosis increases in fibroblasts when glutamine-dependent anaplerosis is impaired, consistent with studies in cancer cells²⁵. Mechanistically, these data support a model in which under low glutamine conditions, PC-mediated anaplerosis maintains sufficient TCA cycle activity to sustain fibroblast growth and protein translation when anabolic precursors such as αKG are diverted into the synthesis of NEAAs. Interestingly, the αKG-derived salt ornithine alpha-ketoglutarate is known to improve wound healing in patients with injuries and burns³⁶; conditions under which glutamine levels drop significantly¹⁴. Consistent with the present findings, it has been suggested that the beneficial effects of αKG on wound healing are mediated at least in part through the replenishment of glutamine and other NEAA pools³⁷.

In addition to supporting protein translation, the present data also show that PC-mediated TCA cycle anaplerosis supports transcription of collagens under low glutamine conditions. This correlated with the ability of PC-mediated anaplerosis to maintain H3K27 acetylation at the Col1a1 locus. H3K27 acetylation was recently shown to regulate Col1a1 transcription in a fibrosis model³⁸. The finding that Col1a1 mRNA expression is reduced in PC-deleted cells cultured in low glutamine is consistent with this model. Reciprocally, repressive H3K27 trimethylation at the Col1a1 locus was increased in cells with PC deletion in low glutamine medium, indicating that PC-mediated anaplerosis contributes to regulating the balance of histone acetylation and methylation. These findings support the emerging concept that the gene expression program of tumor-associated fibroblasts can be regulated by metabolism-driven epigenetic reprogramming^{39, 40}. Unlike the recently reported immunosuppressive role of selective deletion of Col1a1 in PDAC⁴¹, the dependence of fibroblasts on PC is not restricted to Col1a1 expression but is also detected in the expression of other collagens and ECM proteins. Thus, PC is required to produce the collective mix of proteins that contribute to the ECM by supporting fibroblast transcription and translation and may therefore also be required for fibroblast-mediated tumor support in more complex model systems.

Then, the potential source of the pyruvate that is carboxylated by fibroblast-PC to support tumor desmoplasia in vivo was investigated. When both extracellular glutamine and glucose concentrations are low, lactate can be used to maintain collagen production in fibroblasts in a PC-dependent fashion. Lactate concentrations in healing wounds can rise to more than 10 mM, and comparable lactate accumulation in the tumor microenvironment has been reported across studies and cancer types³¹. Lactate is oxidized in wounds and can fuel the TCA cycle in whole tumors^{32, 42}, but so far it has remained unclear which cell type in the tumor microenvironment utilizes lactate. Tumor-associated fibroblasts have been reported to secrete lactate under standard culture conditions^{43, 44} and are thus considered to contribute to, rather than utilize, the lactate accumulating in tumors. However, the transport of lactate across the plasma membrane is dependent on its intra and extracellular concentration and is coupled to the proton gradient, and these factors can differ in tumors and culture systems. The present data indicate that lactate can be taken up by fibroblasts and contribute to the cellular pyruvate pool, independent of the concentration of glucose and glutamine in the culture media. This is consistent with the rapid exchange flux of lactate and pyruvate which has also been observed in vivo^{32, 45}. When TCA cycle anaplerosis from glutamine is limited, lactate-derived pyruvate can be used for anaplerosis in a PC-dependent fashion. Lactate consumption and contribution to the TCA cycle has also been reported in ovarian cancer-associated fibroblasts and in mesenchymal stem cells^{23, 40}. While differences in fibroblasts across tissues might exist, these data suggest that fibroblasts can contribute to lactate consumption observed in tumors and might utilize lactate accumulating in tumors and healing wounds to fuel ECM synthesis. Based on the recently reported effects of lactate on other stromal cell types, the data further support the idea that lactate accumulation in the tumor microenvironment can promote a stromal regenerative response¹³.

Glucose and glutamine are among the highest consumed nutrients by proliferating cells²². As a consequence, glucose and glutamine concentrations in commonly used cell culture media (20 mM and 2 mM, respectively, as used herein) are about fourfold higher than found in human plasma (5 mM and 0.5 mM)¹². The finding that 0.4 mM glutamine (20% Gln) can be limiting for primary fibroblast collagen production in vitro indicates that physiological glutamine levels might be limiting for ECM synthesis. However, unlike in standard cell culture experiments, nutrients are constantly exchanged through the vasculature in healthy tissues in vivo, and are likely maintained at a concentration supportive of essential cellular functions, including ECM production by fibroblasts. In contrast, fresh wounds and most tumor types are poorly vascularized, resulting in reduced nutrient delivery and accumulation of metabolic waste products⁴⁶. Thus, the cell culture experiments performed herein partially mimic the stromal regenerative response observed in many tumors and healing wounds¹³. Impairments in functional vasculature, coupled with the high proliferative activity of cancer cells, generally led to the assumption that glucose and glutamine are depleted in the microenvironment of tumors, relative to levels in plasma and healthy tissues, while lactate accumulates substantially. The reduction of glucose and glutamine and accumulation of lactate have been confirmed in extracts of various tumor types, tumor models and species^{15-17, 31}. The requirement of PC-mediated TCA cycle anaplerosis for collagen production only under glutamine-deprived conditions in vitro suggests that in tumor models glutamine levels are at least at some point limiting for fibroblast collagen synthesis.

TGFβ-mediated stimulation of fibroblast growth and matrix production makes it a target for strategies to impair fibrotic reactions. However, in fibroblasts, TGFβ-stimulated collagen synthesis depends on glutamine availability. While TGFβ/Smad4 signaling promotes anaplerosis from glutamine¹¹, it suppresses anaplerosis from pyruvate via PC, resulting in depletion of TCA cycle intermediates when extracellular glutamine is limiting. Thus, TGFβ renders the TCA cycle dependent on anaplerosis from glutamine. The resulting coupling of matrix production to glutamine availability in TGFβ-stimulated fibroblasts supports collagen synthesis when glutamine is abundant, inducing fibroblasts to store glutamine-derived carbon and nitrogen in the form of highly reduced proline as part of collagen proteins. When glutamine becomes limiting, this coupling could preserve glutamine for parenchymal cells such that organ-specific functions can be maintained. Under such conditions, cancer cells can digest and take up previously laid down ECM proteins to support NEAA biosynthesis and energy homoeostasis⁴⁷. Thus, fibroblast-derived ECM could serve as a sink for carbon, nitrogen and electrons in the tumor microenvironment. As fibroblasts constantly remodel the ECM, this sink might also be used by tumor-associated fibroblasts themselves⁴⁸. Under physiological conditions of a healing wound, the TGFβ-induced coupling of fibroblast collagen production to the glutamine supply could be important to prevent connective tissue regeneration before vascularization is restored.

The regulation of PC expression by TGFβ also suggest that inhibiting TGFβ receptor signaling could potentially increase tumor-associated fibroblast production of collagen through derepressing PC expression and thereby enhance desmoplasia. An interesting correlate of this is that TGFβ-induced repression of PC is Smad4 dependent. Lau et al. (2020) recently reported that in PDAC, tumor cell growth in 3D culture or xenografts is dependent on PC activity. By preventing TGFβ-induced suppression of PC, SMAD4 deletion that is present in 50% of human PDAC may confer a metabolic advantage to PDAC cells when glutamine is limited.

In conclusion, the methods described herein establish TCA cycle anaplerosis as a critical determinant for ECM production in fibroblasts. Specifically, evidence that PC-mediated anaplerosis in tumor-associated fibroblasts contributes to tumor fibrosis and growth is provided. This requirement for PC expression only when glutamine levels are low represents a potential unique vulnerability of fibroblasts that when targeted therapeutically might allow reducing tumor desmoplasia selectively in glutamine-depleted tumors while not affecting the synthesis of ECM in normal tissue.

### Example 8: Methods

### Cell Culture

NIH-3T3 cells were obtained from ATCC (CRL-1658); 293 T cells were obtained from ATCC (CRL-3216). PSCs were isolated from C57BL/6 mice by differential centrifugation as previously described⁴⁹ and their mesenchymal origin was validated by analyzing the expression of various mesenchymal and epithelial markers. MFBs were isolated from FVB/N mice by differential centrifugation as previously described⁵⁰. DB7 mouse breast cancer cells were obtained from Alexander Borowsky (University of California Davis Comprehensive Cancer Center, Sacramento, CA, USA) through an MTA to Ohio State University (OSU). KPC (Kras^{LSL-G12D}; Trp53^{LSL-R172H}; Pdx1-Cre) mouse PDAC cells were a gift from Scott Lowe (Memorial Sloan Kettering Cancer Center. (MSKCC)). All cells were cultured at 37° C. in 5% carbon dioxide (CO₂) and 20% oxygen (O₂) and were maintained in Dulbecco's Modified Eagles medium (DMEM) (25 mM D-glucose, 2 mM L-glutamine) supplemented with 10% fetal bovine serum (FBS) (Gemini), 100 units/ml penicillin and 100 μg/ml streptomycin. Primary PSC and MFB cells were kept in culture for no more than ten passages. Experiments with iCAFs and myCAFs were performed as previously described²⁹. In brief, qPSCs were generated by culturing PSCs in a dome of GFR Matrigel (Corning) in DMEM; iCAFs were generated by culturing PSCs in a dome of GFR Matrigel in DMEM conditioned by KPC cells for 48 h; myCAFs were generated by monolayer culture of PSCs. Differentiation was validated by analyzing the expression of iCAF/myCAF markers²⁹. For experiments with different glutamine concentrations, cells were seeded in regular DMEM and the medium was changed 5-6 hours later or the following day to glucose and glutamine-free DMEM supplemented with 10% dialyzed FBS (Gemini) and 20 mM D-glucose, 2 mM L-glutamine (100%), 0.4 mM L-glutamine (20%) or 0.2 mM L-glutamine (10%). All media were prepared by the Media Preparation Facility at MSKCC. Cells were verified as mycoplasma-free by the MycoAlert Mycoplasma Detection Kit (Lonza).

### Growth Experiments

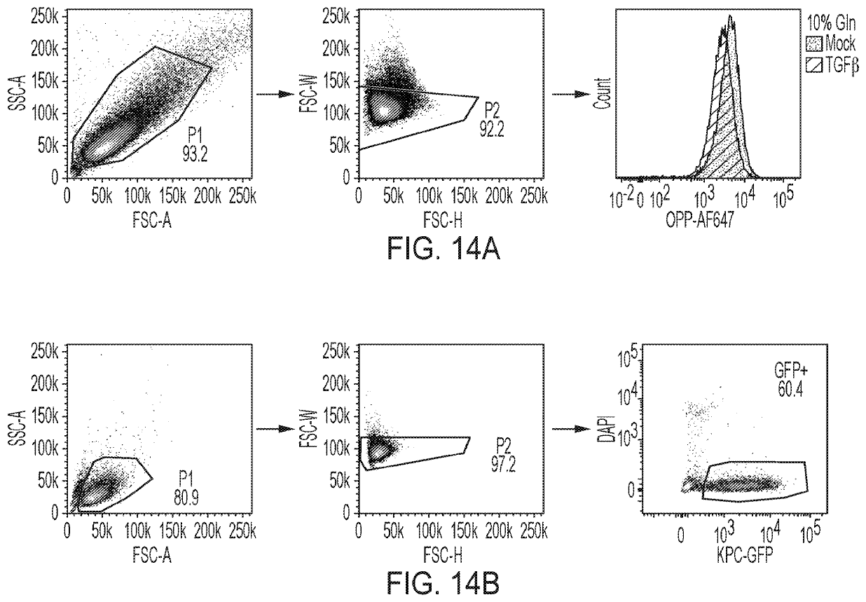

A total of 0.2×10⁵ cells/well were plated in 24-well culture plates and media was changed and treatments started 5-6 hours later. Cell numbers at the start of treatment (day 0 “d 0”) and 1, 2 and 3 days later were counted using the Multisizer 4e (Beckman) and normalized to the cell number at d 0. In co-culture assays, a fraction of the cell population was analyzed by flow cytometry for GFP expression, and the rest counted with the Multisizer 4e. The number of KPC-GFP cells was determined by multiplying the fraction of GFP+ cells with the total number of cells. The gating strategy is shown in FIG. 14B.

### Chemicals

TGFβ-1 was purchased from Peprotech; amino acids (L-asparagine, L-glutamine, proline), cell-permeable metabolites (L-proline methyl ester hydrochloride, L-glutamic acid dimethyl ester hydrochloride, dimethyl 2-oxoglutarate), sodium lactate and L-methionine sulfoximine were purchased from Sigma; CB839 was purchased from Selleck; AZD3965 was purchased from MedChem Express; sodium oxamate was purchased from Cayman Chemical; stable isotopes ([U-¹³C]glucose, [3,4-¹³C]glucose, [U-¹³C]lactate, [1-¹³C]lactate) were purchased from Cambridge Isotope Laboratories. An equivalent amount of solvent (dimethylsulfoxide (DMSO) or water) was added to control samples to control for any solvent-based effects.

### Ectopic Gene Expression and CRISPR/Cas9-Mediated Gene Deletion

Human PC cDNA plasmid was obtained from DNASU. (HsCD00436386). Guide RNAs targeting murine Glul and PC were designed using GuideScan (guidescan.com/) and cloned into pLentiCRISPRv2 (Addgene 52961). The following guide sequences were used:

(Glul sg4)
(SEQ ID NO: 1)
TCGCGCCTACGATCCCAAGG,
(Glul sg6)
(SEQ ID NO: 2)
TGGGATCGTAGGCGCGAATG,
(PC sg1)
(SEQ ID NO: 3)
GCACGCACGAAACACTCGGA,
(PC sg2)
(SEQ ID NO: 4)
TAGGCTTATACTCCAGACGC,
(PC sg4)
(SEQ ID NO: 5)
AAGTTCCAAACAGTTCGAGG,
(PC sg5)
(SEQ ID NO: 6)
GTTCATTGGTCCAAGCCCAG.

Smad4 and Rosa26 targeting guides (Ctrl sg) were described before¹¹. Lentiviral particles were produced in 293 T cells by using psPAX2 and pCMV-VSV-G packaging plasmids (Addgene). Viral supernatant was collected after 48 hours, passed through a 0.45 μm nylon filter and used to transduce NIH-3T3 cells in the presence of 8 μg/mL polybrene (Sigma) overnight. Cells were subjected to puromycin (2 pg/mL, Sigma) or blasticidin (10 pg/mL, Invivogen) antibiotic selection the following day. Polyclonal cell populations were used for the experiments.

### Western Blot

Lysates were generated by incubating cells or ground tumors in RIPA buffer (Millipore); 20-30 pg of cleared lysate were analyzed by SDS-polyacrylamide gel electrophoresis (SDS-PAGE) as previously described¹¹. The following primary antibodies were used: Vinculin (1:5,000 dilution; Sigma, V9131), 3-Actin (1:5,000; Sigma, A5441), Collagen I (1:500; Abcam, ab21286), Fibronectin (1:1,000; Abcam, ab2413), Smad4 (1:200; Santa Cruz, sc-7966), Smad2 p-S465/467 (1:1,000; Cell Signaling, 3108 S), Smad2/3 (1:1,000; Cell Signaling, 3102 S), GCN2 p-T899 (1:1,000; Abcam, ab75836), GCN2 (1:1,000; Cell Signaling, 3302 S), ATF4 (1:200; Santa Cruz, sc-200), PC (1:1,000; Novus, NBP1-49536), S6K (1:1,000; Cell Signaling, 2708 S), GLUL (1:1,000; Sigma, G2781), SMA (1:1,000; Millipore, CBL171). The following secondary antibodies were used: anti-rabbit horseradish peroxidase (HRP) (1:5,000; GE, NA934V), anti-mouse HRP (1:5,000; GE, NA931). Quantification of band intensities of collagen I relative to 3-actin or vinculin in tumor allograft experiments was performed with Image Lab software v6.0 (Bio-Rad).

### Translation Assays

Cells were treated as indicated and incubated with 20 μM O-propargyl-puromycin (OPP, Thermo Scientific) for the last 1 hour of the experiment. Cells were harvested by trypsinization and fixed with methanol at −20° C., followed by permeabilization with 0.5% Triton X-100 in phosphate-buffered saline (PBS). Cells were stained using the Click-iT Plus Alexa Fluor 647 Picolyl Azide Toolkit (Thermo Scientific) according to the manufacturer's instructions and analyzed by flow cytometry. The gating strategy is shown in FIG. 14A.

### ECM Extraction and Collagen Staining

Culture plates were coated with 0.1% gelatin (Sigma) and treated with 1% glutaraldehyde (Sigma) and 1 M ethanolamine (Sigma). Confluent NIH-3T3 cells, MFBs or PSCs were grown for 6 days on coated plates in the presence of 50 M sodium ascorbate (Sigma) and treated as indicated, and the medium was replaced every other day. Plates were decellularized with 20 mM ammonium hydroxide/0.5% Triton-X 100 for 5 min on a rotating platform. Three times the volume of PBS was added, and ECM was equilibrated overnight at 4° C., followed by four additional PBS washes. For western blot, preheated sample buffer supplemented with 1 mM dithiothreitol (DTT) was added, ECM was scraped off and boiled, and proteins were separated by SDS-PAGE followed by immunoblotting. To measure collagen abundance, fibroblast-derived ECM was stained with the Picro Sirius Red Stain Kit (Abcam) according to the manufacturer's instructions. The stain was extracted with 0.1 M sodium hydroxide (NaOH) and optical density was measured at 550 nm using a microplate reader. Differences in cell number were controlled for by growing cells on separate plates under the same experimental conditions.

### Spheroid Outgrowth

Spheroids were generated by plating 1×10⁴ KPC cells in ultra-low attachment spheroid microplates (Corning). The next day, spheroids were transferred to 24-well plates containing synthetic ECM or fibroblast-derived ECM using a P1000 pipette at one spheroid per well. Synthetic ECM was generated by gelating different concentrations of high-concentration rat tail collagen I (Corning) and growth-factor reduced Matrigel (Corning) at a final concentration of 20% in a 37° C. incubator for 1 hour. Spheroids were cultured on top of fibroblast-derived or synthetic ECM in DMEM with 10% FBS and were imaged 2-3 hours after transfer on ECM (d 0) and the three following days with a Zeiss AxioCam microscope. Spheroid area, including outgrowing cells, was quantified manually in Fiji (v2.0).

### Stable Isotope Labelling and Metabolite Extraction

Cells were plated in six-well cell culture plates at a concentration aimed to reach 0.5-1×10⁶ cells at the time of harvest. For quantification of relative metabolite abundance, cells were cultured with glutamine-free DMEM containing different concentrations of L-glutamine and supplemented with 10% dialyzed FBS and in some experiments were treated with TGFβ for 48 hours. For the [U-¹³C]and [3,4-¹³C]glucose tracing experiments, cells were cultured as above, and in the last 8 hours media was replaced with DMEM without D-glucose and L-glutamine supplemented with 2 mM or 0.2 mM L-glutamine and 10 mM [U-¹³C]or [3,4-¹³C]D-glucose (Cambridge Isotope Laboratories) and 10% dialyzed FBS. For the [U-¹³C]and [1-¹³C]lactate tracing experiments, cells were cultured in 2 or 0.2 mM L-glutamine, 10 or 1 mM D-glucose in the presence or absence of 10 mM sodium lactate, and in the last 8 hours media was replaced with DMEM without D-glucose and L-glutamine supplemented with 2 mM or 0.2 mM L-glutamine, 10 or 1 mM D-glucose, 10 mM [U-¹³C]or [1-¹³C]sodium lactate (Cambridge Isotope Laboratories) and 10% dialyzed FBS. For relative quantification of metabolites by gas chromatography-mass spectroscopy (GC-MS), cells were washed briefly with PBS, which was then fully aspirated and metabolism was quenched by immediately adding 1 mL of 80:20 methanol:water stored at −80° C. containing 20 μM deuterated 2-hydroxyglutarate (d5-2HG) as an internal standard. For stable isotope tracing experiments, metabolism was quenched without the PBS washing step by adding 1 mL of 80:20 methanol:water as above. After overnight incubation at −80° C., the resulting extracts were scraped on dry ice, transferred into a 1.5 mL centrifuge tube, and centrifuged at 20,000 g for 20 minutes at 4° C. The supernatants were collected in clean tubes and dried in a vacuum evaporator (Genevac EZ-2 Elite) for 2 hours.

### Stable Isotope Labelling of Cellular Protein and ECM

For [U-¹³C]glucose tracing into proteinogenic amino acids, cells were cultured in 10% Gln in the presence or absence of TGFβ for 48 hours. In the last 24 hours, the media including all treatments was replaced with DMEM without L-glutamine and D-glucose and supplemented with 0.2 mM L-glutamine and 10 mM [U-¹³C]D-glucose (Cambridge Isotope Laboratories) and 10% dialyzed FBS. For [U-¹³C]lactate tracing into ECM, confluent cells were cultured in 10% Gln in the presence of 10 mM [U-¹³C]sodium lactate for 6 days. The media was replaced every other day. ECM was decellularized as described above. Cells and ECM were washed with PBS, and proteins were precipitated with methanol:chloroform:water (1:1:1). The interphase was washed with methanol, and the dried pellet was subjected to acid hydrolysis with 6 N hydrochloric acid (HCl) and incubation at 95° C. for 16 hours. Samples were cooled to room temperature and centrifuged at 20,000 g for 10 minutes. The cleared supernatant was dried in a vacuum evaporator (Genevac EZ-2 Elite) for 2 hours, and abundance of aspartate, glutamate/glutamine or hydroxyproline isotopologues was measured by GC-MS as described below.

### Measurement of Hydroxyproline Levels in Tumors

Flash frozen tumors were ground to a powder in a cryocup grinder (BioSpec) cooled with liquid nitrogen. Acid hydrolysates were generated from aliquots of 5-10 mg ground tumor by addition of 6 N HCl (100 μL/mg) and incubation at 95° C. for 16 hours. Samples were cooled to room temperature and centrifuged at 20,000 g for 10 minutes; 100 μL supernatant was dried in a vacuum evaporator (Genevac EZ-2 Elite) for 2 hours, and hydroxyproline levels were measured by GC-MS as described below.

### Mass Spectrometry Measurement of TCA Cycle Metabolites and Amino Acids

GC-MS measurements were performed as described before¹¹. Ions used for quantification of metabolite levels were as follows: d5-2HG m/z 354; citrate m/z 465; alpha-ketoglutarate m/z 304; succinate m/z 247; fumarate m/z 245; malate m/z 335; aspartate m/z 232; hydroxyproline m/z 332; proline m/z 216; glutamate m/z 246; glutamine m/z 245; lactate m/z 219; pyruvate m/z 174. All peaks were manually inspected and verified relative to known spectra for each metabolite. Peak identification and integration were done with MassHunter software vB.09 (Agilent Technologies). For relative quantification of cell samples, integrated peak areas were normalized to the internal standard d5-2HG and to the packed cell volume of each sample. Absolute quantification of hydroxyproline in tumor acid hydrolysates was performed against a standard curve of commercial trans-4-hydroxy-L-proline (Sigma). In stable isotope tracing experiments, natural isotope abundance correction was performed with IsoCor software v1.2. Liquid chromatography-mass spectroscopy (LC-MS) measurements were performed as described before¹¹. Peak identification and integration were done based on exact mass and retention time match to commercial standards. Data analysis and natural isotope abundance correction were performed with MassHunter Profinder software v10.0 (Agilent Technologies).

### Mouse Experiments

All animal experiments described adhered to policies and practices approved by Memorial Sloan Kettering Cancer Center's Institutional Animal Care and Use Committee (IACUC) and were conducted as per National Institutes of Health (NIH) guidelines for animal welfare (protocol number 11-03-007, Animal Welfare Assurance number FW00004998). The maximal tumor size/burden permitted by the IACUC (tumor burden may not exceed 10% of the weight of the mouse which is equivalent to a tumor volume of 2.5 cm³ for a 25 g mouse) was not exceeded. Mice were maintained under specific pathogen-free conditions and housed four to five mice per cage at a 12-h light/dark cycle at a relative humidity of 30-70% and room temperature of 22.2±1.1° C., and were allowed free access to food and water. Mice were maintained in individually ventilated polysulfone cages with a stainless-steel wire bar lid and filter top on autoclaved aspen chip bedding. Mice were fed a closed-formula, natural-ingredient, 7-irradiated diet (5053 PicoLab® Rodent Diet 20, Purina LabDiet) which was surface decontaminated using “flash” sterilization (100° C. for 1 minute). Mice were provided reverse-osmosis acidified (pH 2.5 to 2.8, with HCl) water. Cage bottoms were changed weekly, whereas the wire bar lid, filter top and water bottle were changed biweekly.

### Tumor Allograft Experiments

For the PDAC allograft model in immunocompromised mice, 1×10⁵ KPC cells alone or together with 5×10⁵ PSCs were resuspended in 100 μL PBS and injected subcutaneously into the flanks of 8-10-week-old female athymic Nude-Foxn1nu mice (Envigo, 069). For the PDAC allograft model in immunocompetent mice, 5×10⁵ KPC cells alone or together with 5×10⁵ PSCs were resuspended in 100 μL PBS and injected subcutaneously into the flanks of 8-10-week-old female syngeneic C57BL/6 mice (JAX, Strain #000664). For the BRCA allograft model, 5×10⁵ DB7 cells alone or together with 5×10⁵ MFBs were resuspended in 100 μL PBS and injected subcutaneously into the flanks of 8-10-week-old female syngeneic FVB/N mice (JAX, Strain #001800). In one experiment, 5×10⁵ DB7 cells were also injected in 1:1 of 100 μL Matrigel (Coming) and PBS. At the beginning of each experiment, mice were randomly assigned to the different groups. No estimation of sample size was performed before the experiments. Mice were monitored daily, and tumor volume was measured by calipers. Measurements were carried out blindly by members of the MSKCC Antitumor Assessment Core and were taken in two dimensions, and tumor volume was calculated as length×width²×π/6. At the end of the experiment, mice were euthanized with CO₂, and tumors were collected and aliquoted for 10% formalin fixation and/or snap freezing.

### Histology

Tissues were fixed overnight in 10% formalin, dehydrated in ethanol, embedded in paraffin and cut into 5 μm sections. Picrosirius Red staining was performed with the Picro Sirius Red Stain Kit (Abcam) according to the manufacturer's instructions. Masson's trichrome staining was performed with the Masson's Trichrome Stain Kit (Polysciences) according to the manufacturer's instructions. For immunofluorescence staining, sections were de-paraffinized with Histo-Clear II (National Diagnostics) and rehydrated according to the manufacturer's instructions. Antigen retrieval was performed for 40 minutes in citrate buffer pH 6.0 (Vector Laboratories) in a steamer (IHC World). Sections were blocked in 5% bovine serum albumin (BSA) and 5% normal goat serum (Cell Signaling) in Tris-buffered saline (TBS) containing 0.1% Tween-20, and incubated in primary antibodies at 4° C. in a humidified chamber overnight. Sections were incubated in secondary antibody in blocking solution for 1 hour at room temperature and mounted in Vectashield Vibrance Antifade Mounting Medium with 4,6-diamidino-2-phenylindole (DAPI) (Vector Laboratories). The following primary antibodies were used: SMA (1:400; Millipore, CBL171), CK8 (1:200; DSHB, TROMA-I). The following secondary antibodies were used: donkey anti-mouse Alexa-Fluor 488, donkey anti-rat Alexa Fluor 647 (1:1,000; Thermo Scientific).

### Image Acquisition and Analysis

Images were acquired with a Mirax Slide Scanner at 20× (brightfield) or 40× (immunofluorescence) magnification. For analyses of tumor fibrosis, color deconvolution was performed in Fiji and the blue channel (Masson's Trichrome) or red channel (Picrosirius Red) were used for quantification. The threshold was determined manually (Picrosirius Red) or with the Yen method (Masson Trichrome) in Fiji. The threshold (stained) area was quantified as a percentage of the total tumor area. Necrotic areas and tumor edges containing skin were identified on hematoxylin and eosin (H&E)-stained consecutive sections and excluded from the analysis. Fibroblasts in tumors were analysed based on SMA staining in Fiji by subtracting background staining, and thresholding with the Otsu method. The threshold (stained) area was quantified as a percentage of the total tumor area using the same regions as for fibrosis quantification.

### Quantification of Gene Expression

Total RNA was isolated from fibroblasts with Trizol (Life Technologies) according to the manufacturer's instructions, and 1 pg RNA was used for cDNA synthesis using iScript (Bio-Rad). Real-time qPCR analysis was performed in technical triplicates using 1:40 diluted cDNAs and 0.1 μM forward and reverse primers together with Power SYBR Green (Life Technologies) in a QuantStudio 7 Flex (Applied Biosystems). Gene expression was quantified in Microsoft Excel 365 as relative expression ratio using primer efficiencies calculated by a relative standard curve. The geometric mean of the endogenous control genes 18 s, Actb and Rplp0 was used as reference sample.

### RNA Sequencing

Total RNA was isolated with Trizol as above, and libraries were prepared from polyA-selected mRNA using the TruSeq RNAsample preparation kit v2 (Illumina) according to the manufacturer's instructions. Libraries were sequenced using an Illumina HiSeq 4000 generating 150 bp paired-end reads. An average of 65 million reads per sample was retrieved. Adaptor sequences were removed from fastq files with Trimmomatic v.0.36, and trimmed reads were mapped to the Mus musculus GRCm38 reference genome using the STAR aligner v.2.5.2b. Aligned features were counted with featureCounts from the Subread package v.1.5.2 and differential expression was determined using DESeq2 v3.10 from Bioconductor in R v4.1.0.

### Gene Set Enrichment Analysis (GSEA)

GSEA was performed using a preranked gene list based on the log 2 fold change comparing two Ctrl sg samples against a total of four PC-ko samples including PC sg2 (two samples) and PC sg5 (two samples). GSEA 4.1.0 (Broad Institute) was used with 1000 permutations and mouse gene symbols remapped to human orthologs v7.2 (MSigDB).

tRNA Charging Assay

Charging status of the indicated tRNA isodecoders was measured as previously described²⁰. In brief, Trizol-chloroform extracts were precipitated with 2.7× volumes of cold ethanol in the presence of 30 μg GlycoBlue (ThermoFisher) overnight. Samples were resuspended in 0.3 M acetate buffer (pH 4.5) with 10 mM ethylenediamine tetraacetic acid (EDTA) and precipitated overnight. Samples were resuspended in 10 mM acetate buffer with 1 mM EDTA. 2 μg of each RNA sample was treated with 10 mM of either sodium periodate (Sigma) (‘oxidized sample’) or sodium chloride (‘non-oxidized sample’) and incubated for 20 minutes at room temperature in the dark. Reactions were quenched with glucose for 15 minutes. Yeast Phe-tRNA (Sigma) was spiked into each sample, followed by ethanol precipitation. Samples were resuspended in 50 mM Tris buffer (pH 9) and incubated for 50 minutes at 37° C., quenched with acetate buffer and precipitated. Samples were resuspended in RNAse-free water and ligated to a 5′ adenylated DNA adaptor using truncated KQ mutant T4 RNA ligase 2 (New England Biolabs) for 3 hours at room temperature. Reverse transcription was performed with SuperScript IV reverse transcriptase (Thermo Scientific) according to the manufacturer's instructions, with a primer complementary to the DNA adaptor. cDNA samples were subjected to qPCR with tRNA isodecoder-specific primer pairs listed in Supplementary Table 1. Ct values obtained with primers specific for yeast Phe-tRNA were subtracted from Ct values obtained with isodecoder-specific primers. The charged fraction was calculated based on the relative difference between the delta-Ct value of a non-oxidized (representing total) and oxidized (representing charged) sample for each primer pair.

### Chromatin Immunoprecipitation

Cells were crosslinked in 1% formaldehyde (Thermo Scientific) in PBS for 10 minutes at room temperature. After quenching with 2.5 M glycine, cell pellets were collected and stored at −80° C. until further processing. One replicate per sample was collected at a time. After all replicates were collected, cells were lysed and subjected to chromatin shearing with the Covaris sonicator (E220) for 25 minutes. The supernatant was cleared and diluted in the same sonication buffer but without N-lauroylsarcosine; 500 μg extract was subjected to immunoprecipitation with 1 μg H3K27me3 (Cell Signaling, 9733 S) or 2 μg H3K27ac (Active Motif, 39034) antibody or an equivalent amount of IgG control (Santa Cruz, sc-66931 or sc-69786) using Protein G magnetic beads (Thermo Scientific) at 4° C. overnight.

The beads were washed, and DNA was reverse-crosslinked overnight and purified using a PCR purification kit (QIAGEN). ChIPed DNA was quantified by qPCR in technical triplicates using 1:10 diluted cDNAs and 0.1 μM forward and reverse primers together with Power SYBR Green (Life Technologies) in a QuantStudio 7 Flex (Applied Biosystems). Primer pairs used for ChIP-qPCR analysis are listed in Supplementary Table 1. Enrichment was calculated in Microsoft Excel 365 as a percentage of input control using a relative standard curve for each primer pair.

### DNA Motif Analysis

Human SMAD2/3/4 motif position frequency matrices were downloaded from the JASPAR database (2020, 8th release). A sequence spanning the putative promoter regions and transcriptional start site (TSS) of three different human PC isoforms was downloaded from the UCSC genome browser (hg38_dna range=chr11:66847159-66962459). Motif searching was performed using FIMO from the MEME Suite with a cutoff of P<0.0001.

### Statistics

A Student's t-test was applied to compare one variable between two groups. One-way ANOVA was applied to compare one variable between three or more groups. Two-way ANOVA was applied to compare two independent variables between two groups. Correction for multiple comparisons was done using the Holm-Sidak method. Pearson correlation was applied to analyze correlation between data from two groups. Statistical analysis was done in GraphPad Prism 8. Most graphs show the mean+SD with individual data points, unless indicated otherwise in the figure legends.

### Data Availability

RNA sequencing data that support the findings described herein have been deposited into the NCBI Gene Expression Omnibus (GEO) with the accession code GSE169588. Human SMAD2/3/4 motif position frequency matrices can be found on JASPAR with the accession code MA0513.1. USCS genome browser tracks are accessible via the following: genome.ucsc.edu/cgibin/hgTracks?db=hg38&lastVirtModeType=default&lastVirtModeExtra State=&virtModeType=default&virtMode=0&nonVirtPosition=&position=chr1%3A66847 371%2D66-973751&hgsid=1152675631_ClzjvmWXzEUYlG9AcUArh1LlTkiY.

### REFERENCES

1. Kalluri, R. The biology and function of fibroblasts in cancer. Nat. Rev. Cancer 16, 582-598 (2016).
2. Hanahan, D. & Weinberg, R. A. The hallmarks of cancer. Cell 100, 57-70 (2000).
3. Dvorak, H. F. Tumors: wounds that do not heal. Similarities between tumor stroma generation and wound healing. N. Engl. J. Med. 315, 1650-1659 (1986).
4. Winslow, S., Lindquist, K. E., Edsjö, A. & Larsson, C. The expression pattern of matrix-producing tumor stroma is of prognostic importance in breast cancer. BMC Cancer 16, 841 (2016).
5. Lim, S. Bin, Tan, S. J., Lim, W. T. & Lim, C. T. An extracellular matrix-related prognostic and predictive indicator for early-stage non-small cell lung cancer. Nat. Commun. 8, 1734 (2017).
6. Whatcott, C. J. et al. Desmoplasia in primary tumors and metastatic lesions of pancreatic cancer. Clin. Cancer Res. 21, 3561-3568 (2015).
7. Pickup, M. W., Mouw, J. K. & Weaver, V. M. The extracellular matrix modulates the hallmarks of cancer. EMBO Rep. 15, 1243-1253 (2014).
8. Provenzano, P. P. et al. Collagen density promotes mammary tumor initiation and progression. BMC Med. 6, 11 (2008).
9. Hamanaka, R. B. & Mutlu, G. M. Metabolic requirements of pulmonary fibrosis: role of fibroblast metabolism. FEBS J. 1-22 (2021) doi. org/10.1111/febs.15693
10. Nigdelioglu, R. et al. Transforming growth factor (TGF)-β promotes de novo serine synthesis for collagen production. J. Biol. Chem. 291, 27239-27251 (2016).
11. Schwörer, S. et al. Proline biosynthesis is a vent for TGFβ-induced mitochondrial redox stress. EMBO J. 39, e103334 (2020).
12. Psychogios, N. et al. The human serum metabolome. PLoS ONE 6, (2011).
13. Schwörer, S., Vardhana, S. A. & Thompson, C. B. Cancer metabolism drives a stromal regenerative response. Cell Metab. 29, 576-591 (2019).
14. Flaring, U. B., Rooyackers, O. E., Wernerman, J. & Hammarqvist, F. Glutamine attenuates post-traumatic glutathione depletion in human muscle. Clin. Sci. 104, 275-282 (2003).
15. Kamphorst, J. J. et al. Human pancreatic cancer tumors are nutrient poor and tumor cells actively scavenge extracellular protein. Cancer Res. 75, 544-553 (2015).
16. Pan, M. et al. Regional glutamine deficiency in tumors promotes dedifferentiation through inhibition of histone demethylation. Nat. Cell Biol. 18, 1090-1101 (2016).
17. Ho, P. C. et al. Phosphoenolpyruvate is a metabolic checkpoint of anti-tumor T. Cell Responses Cell 162, 1217-1228 (2015).
18. Avagliano, A. et al. Influence of fibroblasts on mammary gland development, breast cancer microenvironment remodeling, and cancer cell dissemination. Cancers (Basel). 12, 1697 (2020).
19. Hwang, R. F. et al. Cancer-associated stromal fibroblasts promote pancreatic tumor progression. Cancer Res. 68, 918-926 (2008).
20. Pavlova, N. N. et al. Translation in amino-acid-poor environments is limited by tRNAGln charging. eLife 9, e62307 (2020).
21. Lu, P. D., Harding, H. P. & Ron, D. Translation reinitiation at alternative open reading frames regulates gene expression in an integrated stress response. J. Cell Biol. 167, 27-33 (2004).
22. Zhu, J. & Thompson, C. B. Metabolic regulation of cell growth and proliferation. Nat. Rev. Mol. Cell Biol. (2019) doi.org/10.1038/s41580-019-0123-5
23. Yang, L. et al. Targeting stromal glutamine synthetase in tumors disrupts tumor microenvironment-regulated cancer cell growth. Cell Metab. 24, 685-700 (2016).
24. Francescone, R. et al. Netrin G1 promotes pancreatic tumorigenesis through cancer associated fibroblast driven nutritional support and immunosuppression. Cancer Discov. CD-20-0775 (2020) doi. org/10.1158/2159-8290.CD-20-0775
25. Cheng, T. et al. Pyruvate carboxylase is required for glutamine-independent growth of tumor cells. Proc. Natl Acad. Sci. USA 108, 8674-8679 (2011).
26. Sellers, K. et al. Pyruvate carboxylase is critical for non-small-cell lung cancer proliferation. J. Clin. Invest. 125, 687-698 (2015).
27. Lau, A. N. et al. Dissecting cell-type-specific metabolism in pancreatic ductal adenocarcinoma. eLife 9, e56782 (2020).
28. Davidson, S. M. et al. Environment impacts the metabolic dependencies of ras-driven non-small cell lung cancer. Cell Metab. 23, 517-528 (2016).
29. Öhlund, D. et al. Distinct populations of inflammatory fibroblasts and myofibroblasts in pancreatic cancer. J. Exp. Med. jem.20162024 (2017) doi.org/10.1084/jem.20162024
30. Schvartzman, J. M., Thompson, C. B. & Finley, L. W. S. Metabolic regulation of chromatin modifications and gene expression. J. Cell Biol. jcb.201803061 (2018) doi.org/10.1083/jcb.201803061
31. Goveia, J. et al. Meta-analysis of clinical metabolic profiling studies in cancer: challenges and opportunities. EMBO Mol. Med. 8, 1134-1142 (2016).
32. Hui, S. et al. Glucose feeds the TCA cycle via circulating lactate. Nature 551, 115-118 (2017).
33. Marin-Valencia, I., Roe, C. R. & Pascual, J. M. Pyruvate carboxylase deficiency: mechanisms, mimics and anaplerosis. Mol. Genet. Metab. 101, 9-17 (2010).
34. Cappel, D. A. et al. Pyruvate-carboxylase-mediated anaplerosis promotes antioxidant capacity by sustaining TCA cycle and redox metabolism in liver.

Cell Metab. 29, 1291-1305.e8 (2019).
35. Groen, A. K., Roermund van, C. W. T., Vervoorn, R. C. & Tager, J. M. Control of gluconeogenesis in rat liver cells. Biochem. J. 237, 379-389 (1986).
36. Uzgare, A., Sandra, K., Zhang, Q., Cynober, L. & Barbul, A. Ornithine alpha ketoglutarate enhances wound healing. J. Am. Coll. Surg. 209, S72 (2009).
37. Cynober, L., Lasnier, E., Le Boucher, J., Jardel, A. & Coudray-Lucas, C. Effect of ornithine a-ketoglutarate on glutamine pools in burn injury: evidence of component interaction. Intensive Care Med. 33, 538-541 (2007).
38. Williams, L. M. et al. Identifying collagen VI as a target of fibrotic diseases regulated by CREBBP/EP300. Proc. Natl Acad. Sci. USA. 117, 20753-20763 (2020).
39. Eckert, M. A. et al. Proteomics reveals NNMT as a master metabolic regulator of cancer-associated fibroblasts. Nature 569, 723-728 (2019).
40. Bhagat, T. D. et al. Lactate-mediated epigenetic reprogramming regulates formation of human pancreatic cancer-associated fibroblasts. eLife 8, e50663 (2019).
41. Chen, Y. et al. Type I collagen deletion in αSMA+myofibroblasts augments immune suppression and accelerates progression of pancreatic cancer. Cancer Cell 1-18 (2021) doi.org/10.1016/j.ccell.2021.02.007
42. Amaral, J. F., Shearer, J. D., Mastrofrancesco, B., Gann, D. S. & Caldwell, M.

D. Can lactate be used as a fuel by wounded tissue?Surgery 100, 252-261 (1986).
43. Becker, L. M. et al. Epigenetic reprogramming of cancer-associated fibroblasts deregulates glucose metabolism and facilitates progression of breast cancer. Cell Rep. 31, 107701 (2020).
44. Knudsen, E. S., Balaji, U., Freinkman, E., McCue, P. & Witkiewicz, A. K.

Unique metabolic features of pancreatic cancer stroma: relevance to the tumor compartment, prognosis, and invasive potential. Oncotarget 7, 78396-78411 (2016).
45. Quek, L.-E., Liu, M., Joshi, S. & Turner, N. Fast exchange fluxes around the pyruvate node: a leaky cell model to explain the gain and loss of unlabelled and labelled metabolites in a tracer experiment. Cancer Metab. 4, 13 (2016).
46. Vaupel, P., Kallinowski, F. & Okunieff, P. Blood flow, oxygen and nutrient supply, and metabolic microenvironment of human tumors: a review. Cancer Res. 49, 6449-6465 (1989).
47. Olivares, O. et al. Collagen-derived proline promotes pancreatic ductal adenocarcinoma cell survival under nutrient limited conditions. Nat. Commun. 8, 16031 (2017).
48. Zhu, Z. et al. Tumor-reprogrammed stromal BCAT1 fuels branched-chain ketoacid dependency in stromal-rich PDAC tumors. Nat. Metab. 2, 775-792 (2020).
49. Jesnowski, R. et al. Immortalization of pancreatic stellate cells as an in vitro model of pancreatic fibrosis: deactivation is induced by matrigel and N-acetylcysteine. Lab. Investig. 85, 1276-1291 (2005).
50. Soule, H. D. & McGrath, C. M. A simplified method for passage and long-term growth of human mammary epithelial cells. Vitr. Cell. Dev. Biol. 22, 6-12 (1986).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A method of inhibiting collagen synthesis by a fibroblast, the method comprising contacting the fibroblast with an antagonist of pyruvate carboxylase (PC) and an antagonist of glutaminase.

2. The method of claim 1, wherein the antagonist of PC is a small molecule, a nucleic acid, a polypeptide, or a protein.

3. The method of claim 1 or claim 2, wherein the antagonist of glutaminase is a small molecule, a nucleic acid, a polypeptide, or a protein.

4. The method of any one of claims 1-3, wherein the fibroblast is in a nutrient-replete environment.

5. The method of any one of claims 1-4, wherein the fibroblast is characterized by cell hyperproliferation and/or collagen hyperproduction.

6. The method of any one of claims 1-5, wherein the antagonist of PC decreases the level of acetylated H3K27 at a collagen gene enhancer.

7. The method of any one of claims 1-6, wherein the fibroblast is associated with pulmonary fibrosis, liver fibrosis, and/or cancer.

8. The method of any one of claims 1-7, wherein the contacting is in vitro.

9. The method of any one of claims 1-7, wherein the contacting is in vivo.

10. A method of treating a fibrotic disorder, the method comprising:
administering to a subject in need thereof an antagonist of pyruvate carboxylase (PC) in an amount effective to inhibit extracellular matrix (ECM) protein production, wherein the subject is also receiving an antagonist of glutaminase.

11. The method of claim 10, wherein the fibrotic disorder is characterized by cell hyperproliferation and/or extracellular matrix protein hyperproduction.

12. The method of claim 11, wherein the fibrotic disorder is characterized by a nutrient-replete environment.

13. The method of any one of claims 10-12, wherein the antagonist of PC is a small molecule, a nucleic acid, a polypeptide, or a protein.

14. The method of any one of claims 10-13, wherein the antagonist of glutaminase is a small molecule, a nucleic acid, a polypeptide, or a protein.

15. The method of any one of claims 10-14, wherein treating the fibrotic disorder results in amelioration of cell hyperproliferation and/or extracellular matrix protein hyperproduction.

16. The method of any one of claims 10-15, wherein the antagonist of PC decreases the level of acetylated H3K27 at a collagen gene enhancer.

17. The method of any one of claims 11-16, wherein the extracellular matrix protein is collagen, elastin, and/or laminin.

18. The method of any one of claims 10-17, wherein the fibrotic disorder is pulmonary fibrosis or liver fibrosis.

19. The method of any one of claims 10-18, further comprising administering the antagonist of glutaminase to the subject.

20. The method of claim 19, wherein administering the antagonist of PC and the antagonist of glutaminase is sequential.

21. A method of treating a fibrotic disorder, the method comprising:
administering to a subject in need thereof an antagonist of glutaminase in an amount effective to inhibit extracellular matrix (ECM) protein production, wherein the subject is also receiving an antagonist of pyruvate carboxylase (PC).

22. The method of claim 21, wherein the fibrotic disorder is characterized by cell hyperproliferation and/or extracellular matrix protein hyperproduction.

23. The method of claim 22, wherein the fibrotic disorder is characterized by a nutrient-replete environment.

24. The method of any one of claims 21-23, wherein the antagonist of PC is a small molecule, a nucleic acid, a polypeptide, or a protein.

25. The method of any one of claims 21-24, wherein the antagonist of glutaminase is a small molecule, a nucleic acid, a polypeptide, or a protein.

26. The method of any one of claims 21-25, wherein treating the fibrotic disorder results in amelioration of cell hyperproliferation and/or extracellular matrix protein hyperproduction.

27. The method of any one of claims 21-26, wherein the antagonist of PC decreases the level of acetylated H3K27 at a collagen gene enhancer.

28. The method of any one of claims 22-27, wherein the extracellular matrix protein is collagen, elastin, and/or laminin.

29. The method of any one of claims 21-28, wherein the fibrotic disorder is pulmonary fibrosis or liver fibrosis.

30. The method of any one of claims 21-29, further comprising administering the antagonist of glutaminase to the subject.

31. The method of claim 30, wherein administering the antagonist of PC and the antagonist of glutaminase is sequential.

32. A method of treating a fibrotic disorder, the method comprising:
administering to a subject in need thereof an antagonist of pyruvate carboxylase (PC) and an antagonist of glutaminase in an amount effective to inhibit extracellular matrix production.

33. The method of claim 32, wherein the fibrotic disorder is characterized by cell hyperproliferation and/or hyperproduction of extracellular matrix protein.

34. The method of claim 33, wherein the fibrotic disorder is characterized by a nutrient-replete environment.

35. The method of any one of claims 32-34, wherein the antagonist of PC is a small molecule, a nucleic acid, a polypeptide, or a protein.

36. The method of any one of claims 32-35, wherein the antagonist of glutaminase is a small molecule, a nucleic acid, a polypeptide, or a protein.

37. The method of any one of claims 32-36, wherein treating the fibrotic disorder results in amelioration of cell hyperproliferation and/or hyperproduction of extracellular matrix protein.

38. The method of any one of claims 32-37, wherein the antagonist of PC decreases the level of acetylated H3K27 at a collagen gene enhancer.

39. The method of any one of claims 33-38, wherein the extracellular matrix protein is collagen, elastin, and/or laminin.

40. The method of any one of claims 32-39, wherein the fibrotic disorder is pulmonary fibrosis or liver fibrosis.

41. A method of inhibiting tumor cell proliferation, the method comprising contacting the tumor cell with an antagonist of pyruvate carboxylase (PC) and an antagonist of glutaminase.

42. The method of claim 41, wherein inhibition of tumor cell proliferation results from reduction of extracellular matrix protein.

43. The method of claim 41 or claim 42, wherein the antagonist of PC is a small molecule, a nucleic acid, a polypeptide, or a protein.

44. The method of any one of claims 41-43, wherein the antagonist of glutaminase is a small molecule, a nucleic acid, a polypeptide, or a protein.

45. The method of any one of claims 41-44, wherein the contacting is in a nutrient-replete environment.

46. The method of any one of claims 41-45, wherein the tumor cell is a fibroblast.

47. The method of any one of claims 41-46, wherein the tumor cell is derived from a carcinoma, a sarcoma, a breast cancer, a pancreatic cancer, a lung cancer, a leukemia, a lymphoma, a brain cancer, a melanoma, a liver cancer, a stomach cancer, a small intestine cancer, a large intestine cancer, a kidney cancer, a uterine cancer, an ovarian cancer, a bladder cancer, or a bone cancer.

48. The method of any one of claims 41-47, wherein the contacting is in vitro.

49. The method of any one of claims 41-47, wherein the contacting is in vivo.

50. A method of treating a cancer, the method comprising:
administering to a subject in need thereof an antagonist of pyruvate carboxylase (PC) in an amount effective to inhibit carcinogenesis, wherein the subject is also receiving an antagonist of glutaminase.

51. The method of claim 50, wherein the cancer is characterized by cell hyperproliferation and/or extracellular matrix protein hyperproduction.

52. The method of claim 51, wherein the cancer is characterized by a nutrient-replete environment.

53. The method of any one of claims 50-52, wherein the antagonist of PC is a small molecule, a nucleic acid, a polypeptide, or a protein.

54. The method of any one of claims 50-53, wherein the antagonist of glutaminase is a small molecule, a nucleic acid, a polypeptide, or a protein.

55. The method of any one of claims 50-54, wherein treating the cancer results in a reduction of proliferation and/or extracellular matrix protein production.

56. The method of any one of claims 50-55, wherein the antagonist of PC decreases the level of acetylated H3K27 at a collagen gene enhancer.

57. The method of any one of claims 51-56, wherein the extracellular matrix protein is collagen, elastin, and/or laminin.

58. The method of any one of claims 50-57, wherein the tumor cell is derived from a carcinoma, a sarcoma, a breast cancer, a pancreatic cancer, a lung cancer, a leukemia, a lymphoma, a brain cancer, a melanoma, a liver cancer, a stomach cancer, a small intestine cancer, a large intestine cancer, a kidney cancer, a uterine cancer, an ovarian cancer, a bladder cancer, or a bone cancer.

59. The method of any one of claims 50-58, further comprising administering the antagonist of glutaminase to the subject.

60. The method of claim 59, wherein administering the antagonist of PC and the antagonist of glutaminase is sequential.

61. A method of treating a cancer, the method comprising:
administering to a subject in need thereof an antagonist of glutaminase in an amount effective to inhibit carcinogenesis, wherein the subject is also receiving an antagonist of pyruvate carboxylase (PC).

62. The method of claim 61, wherein the cancer is characterized by cell hyperproliferation and/or extracellular matrix protein hyperproduction.

63. The method of claim 62, wherein the cancer is characterized by a nutrient-replete environment.

64. The method of any one of claims 61-63, wherein the antagonist of PC is a small molecule, a nucleic acid, a polypeptide, or a protein.

65. The method of any one of claims 61-64, wherein the antagonist of glutaminase is a small molecule, a nucleic acid, a polypeptide, or a protein.

66. The method of any one of claims 61-65, wherein treating the cancer results in a reduction of cell proliferation and/or extracellular matrix protein production.

67. The method of any one of claims 61-66, wherein the antagonist of PC decreases the level of acetylated H3K27 at a collagen gene enhancer.

68. The method of any one of claims 62-67, wherein the extracellular matrix protein is collagen, elastin, and/or laminin.

69. The method of any one of claims 61-68, wherein the tumor cell is derived from a carcinoma, a sarcoma, a breast cancer, a pancreatic cancer, a lung cancer, a leukemia, a lymphoma, a brain cancer, a melanoma, a liver cancer, a stomach cancer, a small intestine cancer, a large intestine cancer, a kidney cancer, a uterine cancer, an ovarian cancer, a bladder cancer, or a bone cancer.

70. The method of any one of claims 61-69, further comprising administering the antagonist of PC to the subject.

71. The method of claim 70, wherein administering the antagonist of glutaminase and the antagonist of PC is sequential.

72. A composition comprising an antagonist of pyruvate carboxylase (PC) and an antagonist of glutaminase.

73. The composition of claim 72, wherein the antagonist of PC is a small molecule, a nucleic acid, a polypeptide, or a protein.

74. The composition of claim 72 or claim 73, wherein the antagonist of glutaminase is a small molecule, a nucleic acid, a polypeptide, or a protein.

75. The composition of any one of claims 72-74, wherein the composition further comprises a nutrient-replete cell culture medium.

76. A kit comprising:
an antagonist of PC;
an antagonist of glutaminase; and
instructions for use of the antagonist of PC and the antagonist of glutaminase.

77. The kit of claim 76, comprising a single container containing the antagonist of PC and the antagonist of glutaminase.

78. The kit of claim 77, wherein the single container is a vial or a syringe.

79. The kit of claim 76, comprising a first container containing the antagonist of PC, and a second container containing the antagonist of glutaminase.

80. The kit of claim 79, wherein the first and the second containers are vials or syringes.

81. The kit of any one of claims 76-80, wherein the instructions are instructions for use of the antagonist of PC and the antagonist of glutaminase in a method according to any one of claims 1-71.


SEQUENCE LISTING (6 sequences)

SEQ ID NO: 1 - DNA, 20 bp, synthetic construct
  misc_feature 1..20: note = Synthetic
  source 1..20: mol_type = other DNA
tcgcgcctacgatcccaagg

SEQ ID NO: 2 - DNA, 20 bp, synthetic construct
  misc_feature 1..20: note = Snythetic
  source 1..20: mol_type = other DNA
tcgcgcctacgatcccaagg

SEQ ID NO: 3 - DNA, 20 bp, synthetic construct
  misc_feature 1..20: note = Synthetic
  source 1..20: mol_type = other DNA
gcacgcacgaaacactcgga

SEQ ID NO: 4 - DNA, 20 bp, synthetic construct
  misc_feature 1..20: note = Synthetic
  source 1..20: mol_type = other DNA
taggcttatactccagacgc

SEQ ID NO: 5 - DNA, 20 bp, synthetic construct
  misc_feature 1..20: note = Synthetic
  source 1..20: mol_type = other DNA
aagttccaaacagttcgagg

SEQ ID NO: 6 - DNA, 20 bp, synthetic construct
  misc_feature 1..20: note = Synthetic
  source 1..20: mol_type = other DNA
gttcattggtccaagcccag